(19) United States
(12) Patent Application Publication
(10) Pub. No.: US 2023/0183739 A1
(43) Pub. Date: Jun. 15, 2023

(54) RECOMBINANT MEASLES VIRUS EXPRESSING PROTEINS OF A PLASMODIUM PARASITE AND THEIR APPLICATIONS

(71) Applicants: INSTITUT PASTEUR, PARIS (FR); CENTRE NATIONAL DE LA RECHERCHE SCIENTIFIQUE, PARIS (FR)

(72) Inventors: Frederic TANGY, LES LILAS (FR); Marie MURA, SILVER SPRING, MD (US); Rogerio AMINO, BAGNEUX (FR); Chetan CHITNIS, PARIS (FR)

(21) Appl. No.: 17/057,235

(22) PCT Filed: May 23, 2019
(86) PCT No.: PCT/IB2019/000703

§ 371 Date: Nov. 20, 2020

Related U.S. Application Data
(60) Provisional application No. 62/675,265, filed on May 23, 2018.

(51) Int. Cl. C12N 15/86; A61K 39/015; A61P 33/06

(52) U.S. Cl. CPC C12N 15/86; A61K 39/015; A61P 33/06; C12N 2760/18444

(57) ABSTRACT

The present invention relates to recombinant measles virus expressing proteins of a Plasmodium parasite and their applications, in particular in inducing preventive protection against a Plasmodium infection. The present invention is directed to recombinant measles virus (MV) expressing (i) at least the circumsporozoite (CS) protein of a Plasmodium parasite or an antigenic fragment thereof, and at least a chimeric antigen of a Plasmodium parasite as defined below, or (ii) at least the CS protein of a Plasmodium parasite or an antigenic fragment thereof, at least a chimeric antigen of a Plasmodium parasite as defined below and at least the reticulocyte-binding protein homologue 5 (RH5) of a Plasmodium parasite or an antigenic fragment thereof, and concerns recombinant infectious virus partides of said MV-malaria able to replicate in a host after an administration. The present invention provides means, in particular nucleic acids, vectors, cells and rescue systems to produce these recombinant infectious virus particles. The present invention also relates to the use of these recombinant infectious virus particles, in particular under the form of a composition, more particularly in a vaccine composition, for the prevention of a Plasmodium infection or for the preventive protection against clinical outcomes of infection by a Plasmodium parasite.

The present invention relates to recombinant measles virus expressing proteins of a Plasmodium parasite and their applications, in particular in inducing preventive protection against a Plasmodium infection. The present invention is directed to recombinant measles virus (MV) expressing (i) at least the circumsporozoite (CS) protein of a Plasmodium parasite or an antigenic fragment thereof, and at least a chimeric antigen of a Plasmodium parasite as defined below, or (ii) at least the CS protein of a Plasmodium parasite or an antigenic fragment thereof, at least a chimeric antigen of a Plasmodium parasite as defined below and at least the reticulocyte-binding protein homologue 5 (RH5) of a Plasmodium parasite or an antigenic fragment thereof, and concerns recombinant infectious virus particles of said MV-malaria able to replicate in a host after an administration. The present invention provides means, in particular nucleic acids, vectors, cells and rescue systems to produce these recombinant infectious virus particles. The present invention also relates to the use of these recombinant infectious virus particles, in particular under the form of a composition, more particularly in a vaccine composition, for the prevention of a Plasmodium infection or for the preventive protection against clinical outcomes of infection by a Plasmodium parasite.

Despite decades of malaria vaccine research, only RTS,S/AS01 vaccine candidate reached Phase III clinical trial to eventually show moderate protection of short duration (Aaby, et al. Lancet 2015, 386(10005):1735-6). This led the World Health Organization to recommend additional pilot studies in three countries with enhanced pharmacovigilance (Greenwood, et al. Lancet 2016, 387(10016):318-9). Nevertheless, these results are encouraging as they establish the feasibility of developing a malaria vaccine. Furthermore, the spread of artemisinin-resistant P. falciparum strains (Blasco, et al. Nat. Med. 2017, 23(8): 917-928) underlines the need for an effective vaccine for sustained protection against malaria. The rationale for malaria vaccine development relies on several observations. First, natural immunity is gradually acquired to severe, life-threatening malaria and then to clinical disease after several years of natural exposure (Riley, et al. Nat. Med. 2013, 19(2):168-78). Nevertheless, this immunity is not sterile and quickly wanes if an individual leaves the endemic area. Continued exposure to parasites is, therefore, required to maintain immunological memory (Triller, et al., Immunity 2017, 47(6):1197-209 e10). Second, transfer of gamma-globulin fractions from semi-immune to naïve humans clears blood stage parasites and mitigates malaria disease (Cohen, et al. Nature 1961, 192:733-7). Finally, inoculation of irradiated attenuated sporozoites can protect humans against infectious challenge, but requires high and frequent doses, and immunity wanes after six months (Ishizuka, et al. Nat. Med. 2016, 22(6): 614-23). Therefore, the induction of long-term memory is critical for sustained vaccine efficacy.

The RTS,S subunit vaccine is based on the Plasmodium falciparum is circumsporozoite (CS) protein, which is expressed during the sporozoite and early liver stages, and is involved in adhesion and invasion of hepatocytes. CS is known as the lead antigen for inclusion in a pre-erythrocytic vaccine candidate. Based on data on efficacy elicited by CS in pre-clinical as well as human challenge models, the CS is considered a “gold standard” that can be used to evaluate different vaccine delivery platforms and prime-boost strategies (Ockenhouse, et al. J. Infect. Dis. 1998, 177(6):1664-73; Stoute, et al. J. Infect. Dis. 1998, 178(4):1139-44; Chuang, et al. PLoS One 2013, 8(2):e55571). The CS is composed of a central and conserved Asparagine-Alanine-Asparagine-Proline (NANP) amino acid repeat sequence, known as the immunodominant B-cell epitope. Indeed, CS-specific antibodies and CD4+ T cell responses were associated with human protection during RTS,S controlled human malaria infection trials (CHMI) (White, et al. PLoS One 2013, 8(4):e61395). However, RTS,S/AS01 did not induce CD8⁺ T cell responses, which can play an important role in parasite elimination in the liver (Radtke, et al. PLoS Pathog. 2015, 11(2):e1004637). Viral vectors are known for their capacity to induce CD8⁺ T cell response but prime-boost strategies with AdCh63 and MVA, which are non-replicative viral vectors, were disappointing (Ockenhouse, et al., PLoS One 2015, 10(7): e0131571; Dunachie, et al. Vaccine 2006, 24(15):2850-9).

The measles virus (MV) vector based vaccine platform offers new opportunities as a replicative but safe viral vector. The general rationale for the use of MV is based on the following arguments: (i) MV is one of the safest and most effective human vaccines, eliciting life-long protective immunity against measles after a single injection; (ii) its production can be easily scaled up at low cost, which is important for developing countries where malaria is endemic; (iii) immunization with MV vector induces both humoral and cellular responses to the transgenes (Lorin, et al. J. Virol. 2004, 78(1):146-57; Guerbois, et al. Virol. 2009, 388(1):191-203; Brandler, et al. PLoS Negl. Trop. Dis. 2007, 1(3):e96; Liniger, et al. Vaccine 2009, 27(25-26):3299-305; Stebbings, et al. PLoS One 2012, 7(11):e50397); (iv) MV genome can integrate up to six kb in additional transcription units, allowing the expression of several malaria antigens; (v) a phase I clinical trial with a recombinant MV vaccine expressing chikungunya virus-like particles showed that, unlike non replicative viral vector platforms, there was no impact of pre-existing immunity against measles vector (Ramsauer, et al. Lancet Infect. Dis. 2015,15(5):519-27). This important point has also been confirmed in the ongoing phase II clinical trial; (vi) In 2016, about 85% of the world's children received one dose of measles vaccine by their first birthday through routine health services. A recombinant measles-malaria vaccine could therefore easily be integrated in vaccination schedules.

The European patent EP2427201 and the US patent US9308250 disclose a combined measles-malaria vaccine containing different attenuated recombinant measles-malaria vectors comprising a heterologous nucleic acid encoding several Plasmodium falciparum antigens, e.g. the CS malaria antigen or malaria antigen d42 fragment of MSP1.

The inventors generated a recombinant MV expressing polypeptides from the CS protein of Plasmodium berghei (Pb) or Plasmodium falciparum (Pf) to establish proof of concept for the use of measles vector to express malaria antigens. In the CSPb model, the inventors demonstrated that rMV-CSPb was able to induce sterile protection of mice or at least protect them from severe symptoms with reduced blood parasitemia. In the CSPf model, rMV-CSPf induced immunogenicity as a Th1 profile and was maintained from 3 weeks up to, at least, 4 months after the second immunization. Furthermore, the inventors showed the induction of CD4⁺ and CD8⁺ cellular responses. The inventors further explored the use of the MV-based vaccine platform to deliver the CS protein and induce a cellular response as well as high antibody titers with long-term memory. Such a vaccine is key for the development of a malaria vaccine with higher efficacy and long-term protection against P. falciparum malaria in a human, in particular when related to infection.

The inventors achieved the production of vaccines based on recombinant infectious replicative MV recombined with polynucleotides encoding malaria antigens, which are recovered when the recombinant virus replicates in particular in the host after administration. The invention thus relates to a live Malaria vaccine active ingredient based on the widely used measles, in particular measles from the Schwarz strain, pediatric vaccine.

MV is a non-segmented single-stranded, negative-sense enveloped RNA virus of the genus Morbillivirus within the family of Paramyxoviridae. This virus has been isolated in 1954 (Enders, J. F., and T. C. Peebles. 1954. Propagation in tissue cultures of cytopathogenic agents from patients with measles. Proc. Soc. Exp. Biol. Med. 86:277-286), and live-attenuated vaccines have been derived from this virus since then to provide vaccine strains, in particular from the Schwarz strain. Measles vaccines have been administered to hundreds of millions of children over the last 30 years and have proved its efficiency and safety. It is produced on a large scale in many countries and is distributed at low cost. For all these reasons, the inventors used attenuated MVs to generate recombinant MV particles stably expressing particular antigens of Malaria.

The invention thus relates to a chimeric measles virus (MV)-based nucleic acid construct suitable for the expression of heterologous polypeptides, which comprises:
a cDNA molecule encoding a full-length, infectious antigenomic (+) RNA strand of a MV; and
(1) a first heterologous polynucleotide encoding at least the circumsporozoite (CS) protein of a Plasmodium parasite or an antigenic fragment thereof; and
(2) a second heterologous polynucleotide encoding at least a chimeric antigen of a Plasmodium parasite; and

wherein said a chimeric antigen as defined in (2) comprises or consists of the following fragments of (a), (b), (c) and (d) assembled in a fusion polypeptide, wherein the fragments of (a), (b), (c) and (d) elicit a human leukocyte antigen (HLA)-restricted CD8⁺ and/or CD4⁺ T cell response against a Plasmodium parasite, and are directly or indirectly fused in this order:
(a) a fragment of the inhibitor of cysteine protease (ICP) (18-10) of a Plasmodium parasite,
(b) a fragment of the protein Ag45 (11-10) of a Plasmodium parasite,
(c) a fragment of the thrombospondin related anonymous protein (TRAP) of a Plasmodium parasite, and
(d) the protein Ag40 (11-09) of a Plasmodium parasite or a fragment thereof,
   or a chimeric antigen variant thereof, which consists of a chimeric antigen having an amino acid sequence which has at least 90% sequence identity or more than 95% sequence identity or 99% sequence identity with the sequence of the fusion polypeptide consisting of fused fragments of (a), (b), (c) and (d), from which it derives by point mutation of one or more amino acid residues, over its whole length;

wherein the first heterologous polynucleotide is operatively linked, in particular cloned within an additional transcription unit (ATU) inserted within the cDNA molecule; and

wherein the second heterologous polynucleotide is operatively linked, in particular cloned within an ATU inserted within the cDNA molecule at a location distinct from the location of the first linked, in particular cloned heterologous polynucleotide.

The expressions “Plasmodium parasite” and “malaria parasite” are used interchangeably in the present application. They designate every and all forms of the parasite that are associated with the various stages of the parasite cycle in the mammalian, especially human host, including in particular sporozoites, especially sporozoites inoculated in the host skin and present in the blood flow after inoculation, or sporozoites developing in the hepatocytes (liver-stages), merozoites, including especially merozoites produced in the hepatocytes and s merozoites produced in the red-blood cells, or merozoites developing in the red-blood cells (blood-stages). These various forms of the parasite are characterized by multiple specific antigens, many of which are well known and identified in the art and some of which are still unknown and to which no biological function has yet been assigned. The antigens can often be designated or classified in groups by reference to their expression according to the stage of the infection. Plasmodium parasites according to the present invention encompass parasites infecting human hosts and parasites infecting non-human mammals especially rodents and in particular mice. Accordingly, Plasmodium falciparum, Plasmodium vivax, Plasmodium yoelii and is Plasmodium berghei are particular examples of these parasites. Plasmodium cynomolgi and Plasmodium knowlesi are primarily infectious for macaques, but can also cause human infection.

As defined herein, the expression “encoding” defines the ability of the nucleic acid molecules to be transcribed and where appropriate translated for product expression into selected cells or cell lines. Accordingly, the nucleic acid construct may comprise regulatory elements controlling the transcription of the coding sequences, in particular promoters and termination sequences for the transcription and possibly enhancer and other cis-acting elements. These regulatory elements may be heterologous with respect to the polynucleotide sequences of the Plasmodium parasite.

The term “protein” is used interchangeably with the terms “antigen” or “polypeptide” and defines a molecule resulting from a concatenation of amino acid residues. In particular, the proteins disclosed in the application originate from a Plasmodium parasite and are structural proteins that may be identical to native proteins or alternatively that may be derived thereof by mutation, including by substitution (in particular by conservative amino acid residues) or by addition of amino acid residues or by secondary modification after translation or by deletion of portions of the native proteins(s) resulting in fragments having a shortened size with respect to the native protein of reference.

As defined herein, the term “fragment” refers to parts or portions of proteins of a Plasmodium parasite. It can be a fragment of the native antigen of the Plasmodium parasite and especially a truncated version of such native antigen or a modified version thereof as a result of post-translational modifications. Fragments encompassed within the present invention bear epitopes of the native protein suitable for the elicitation of an immune response in a host in particular in a human host, preferably a response that enables the protection against a Plasmodium infection or against a Plasmodium associated disease. Epitopes are in particular of the type of B epitopes involved in the elicitation of a humoral immune response through the activation of the production of antibodies in a host to whom the protein has been administered or in whom it is expressed following administration of the infectious replicative virus particles of the invention. Epitopes may alternatively be of the type of T epitopes involved in elicitation of Cell Mediated Immune response (CMI response) including CD4⁺ or CD8⁺ T epitopes. Fragments may have a size representing more than 50% of the amino acid sequence size of the native protein of the Plasmodium parasite, in particular at least 60%, more particularly at least 70%, preferably at least 80%, more preferably at least 90% or at least 95%. Amino acid sequence identity can be determined by alignment by one skilled in the art using manual alignments or using the numerous alignment programs available.

In a preferred embodiment of the invention, the fragments of (a), (b), (c) and (d) of a chimeric antigen according to the invention are amino acid sequences that comply with the rule of six, i.e. consist of a number of amino acids that is a multiple of six, at least when the sequence of the chimeric antigen is taken as a whole.

The fragments of (a), (b), (c) and (d) of a chimeric antigen according to the invention elicit collectively and/or individually a human leukocyte antigen (HLA)-restricted CD8⁺ and/or CD4⁺ T cell response against a Plasmodium parasite. The expression “HLA-restricted” refers to the capacity for a particular fragment or epitope to have an affinity for this type of HLA molecule. The HLA molecules used in the invention encompass either class I molecules (designated HLA-A, B or C) or class II molecules (designated DP, DQ or DR).

The chimeric antigen of the invention can be synthesized chemically, or produced either in vitro (cell free system) or in vivo after expression of the nucleic acid molecule encoding the antigen in a cell system.

The term “chimeric antigen” is used interchangeably with the term “chimeric polyepitope” and means any polyepitopic polypeptide comprising at least sub-portions of different proteins of a Plasmodium parasite selected among the protein Ag45, the protein Ag40 and the TRAP of a Plasmodium parasite. The chimeric antigen is constructed by fusing directly or indirectly at least the above-defined fragments of (a), (b), (c) and (d), while avoiding the creation of neo-epitopes at the junction of antigens/protective domains constituting the fragments. If necessary, one or more amino acid residues may be introduced in the fusion sequence to avoid the creation of neo-epitopes with high binding affinity to HLA.

As defined herein, the term “directly fused” means that the 3′ end of a fragment of (a), (b), (c) and (d) of a chimeric antigen, or of a particular heterologous polynucleotide is directly linked to the 5′ end of another fragment of (a), (b), (c) and (d) of a chimeric antigen, or of another particular heterologous polynucleotide respectively.

As defined herein, when a fragment of (a), (b), (c) and (d) of a chimeric antigen or a particular heterologous polynucleotide is “indirectly fused” with another fragment of (a), (b), (c) and (d) of a chimeric antigen, or another particular heterologous polynucleotide, it involves the presence of amino acid residue segments which do not read on the native protein of the Plasmodium parasite providing the sequence of the considered fragment or heterologous polynucleotides respectively, i.e. it involves a linker sequence whose amino acid sequence is well known in the art.

A nucleic acid construct according to the invention is in particular a purified DNA molecule, obtained or obtainable by recombination of various polynucleotides of different origins, operably linked together.

The expression “operatively linked” refers to the functional link existing between the different polynucleotides of the nucleic acid construct of the invention such that said different polynucleotides and nucleic acid construct are efficiently transcribed and if appropriate translated, in particular in cells or cell lines, especially in cells or cell lines used as part of a rescue system for the production of chimeric infectious MV particles of the invention or in host cells, especially in human cells. The term “operably” may be used herein as equivalent to “operatively”.

In a particular embodiment of the invention, the construct is prepared by cloning a polynucleotide encoding at least the CS protein of the Plasmodium parasite or the antigenic fragment thereof, at least the above-defined chimeric antigen of the Plasmodium parasite and and optionally at least the RH5 of the Plasmodium parasite or the antigenic fragment thereof. Alternatively, a nucleic acid construct of the invention may be prepared using steps of synthesis of nucleic acid fragments or polymerization from a template, including by PCR.

In a particular embodiment of the invention, the nucleic acid construct complies with the rule of six (6) of the MV genome, i.e. consists of a number of nucleotides that is a multiple of six.

The organization of the genome of MVs and their replication and transcription process have been fully identified in the prior art and are especially disclosed in Horikami S. M. and Moyer S. A. (Curr. Top. Microbiol. Immunol. (1995) 191, 35-50) or in Combredet C. et al (Journal of Virology, November 2003, p11546-11554) for the Schwarz vaccination strain of the virus or for broadly considered negative-sense RNA viruses, in Neumann G. et al (Journal of General Virology (2002) 83, 2635-2662).

The “rule of six” is expressed in the fact that the total number of nucleotides present in a nucleic acid representing the MV(+) strand RNA genome or in nucleic acid constructs comprising same is a multiple of six. The “rule of six” has been acknowledged in the state of the art as a requirement regarding the total number of nucleotides in the genome of the MV, which enables efficient or optimized replication of the MV genomic RNA. In the embodiments of the present invention defining a nucleic acid construct that meets the rule of six, said rule applies to the nucleic acid construct specifying the cDNA encoding the full-length MV (+) strand RNA genome and preferably all inserted sequences when taken collectively. In this regard the rule of six applies to the cDNA encoding the full-length infectious antigenomic (+) RNA strand of the MV possibly and to the polynucleotide contained, in particular cloned into said cDNA and encoding at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof.

Each heterologous polynucleotide may be issued from the fusion of several distinct polynucleotides, each encoding a particular polypeptide, or a particular protein, antigen or an antigenic fragment thereof, of a Plasmodium parasite. For example, the heterologous polynucleotide may be issued from the fusion of polynucleotides each encoding a single protein or antigenic fragment thereof of a Plasmodium parasite, these two polynucleotides being optionally linked within the nucleic acid construct by a linker sequence. A linker sequence is well known in the art and may be a short nucleotide sequence comprising or consisting in a regulatory sequence of the measles virus.

In a preferred embodiment of the invention, the nucleic acid construct further comprises a third heterologous polynucleotide encoding at least the reticulocyte-binding protein homologue 5 (RH5) of a Plasmodium parasite or an antigenic fragment thereof, wherein said third heterologous polynucleotide is directly fused or indirectly fused to the first heterologous polynucleotide. The RH5 of a Plasmodium falciparum has been reported to target merozoite ligand that mediates erythrocyte invasion (Ouattara et al., Vaccines 2015:60, 930-936; Ord et al., Malaria J. 2014, 13: 326).

The above-mentioned definition regarding the terms “directly fused” and “indirectly fused” applies to said third heterologous polynucleotide. In particular, the third heterologous polynucleotide may be indirectly fused to the first heterologous polynucleotide by means of an intergenic sequence. An example of the nucleotide sequence of the polynucleotide encoding an intergenic sequence as well as the amino acid sequence of said intergenic sequence is disclosed as SEQ ID NO: 5 and SEQ ID NO: 6 respectively.

The polynucleotides encoding at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof are cloned into an ATU (Additional Transcription Unit) inserted in the cDNA of the MV. ATU sequences are known from the skilled person and comprise, for use in steps of cloning into cDNA of MV, cis-acting sequences necessary for MV-dependent expression of a transgene, such as a promoter of the gene preceding, in MV cDNA, the insert represented by the polynucleotide encoding at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof, and a multiple cloning sites cassette for insertion of said polynucleotide.

The ATU may be further defined as disclosed by Billeter et al. in WO 97/06270. An ATU may also be defined as multiple cloning cassette inserted within the cDNA of the MV, in particular between the N-P intergenic region of the MV genome, and/or between the intergenic H-L region of the MV genome. An ATU may contain cis-acting sequences necessary for the transcription of the P gene of MV. The ATUs provided at distinct locations in MV cDNA may be identical regarding their nucleic acid sequence. ATUs are generally localized between two CTT codons corresponding respectively to the start and stop codons used by the polymerase. ATUs may further comprise a ATG and a TAG codons corresponding respectively to the start and stop codons for translation of the heterologous polynucleotide cloned within the ATU. Alternatively, ATUs are localized between a ATG and a TAG codons corresponding respectively to the start and stop codons for translation of the heterologous polynucleotide cloned within the ATU. ATUs may further comprise a ATG and a TAG codons corresponding respectively to the start and stop codons for translation of the heterologous polynucleotide cloned within the ATU. In a preferred embodiment of the invention, an ATU is a polynucleotide comprising or consisting of SEQ ID NO: 7.

SEQ ID NO: 7

SEQ ID NO: 7 is an ATU sequence localized within the cDNA molecule encoding a full-length antigenomic (+) RNA strand of a measles virus. CTT codons corresponding respectively to the start and stop codons of the polymerase are in bold. ATG and TAG codons corresponding to the start and stop codons for translation of the heterologous polynucleotide cloned within the ATU are underlined.

CTTAGGAACCAGGTCCACACAGCCGCCAGCCCATCAacgcgtacgAT G*TAGgcgcgcagcgcttagacgtctcgcgaTCGATACTAGTACAACCTAAATCCATT ATAAAAAACTT wherein the * corresponds to the codon-optimized sequence of a specific heterologous polynucleotide encoding at least a protein of a Plasmodium parasite.

An ATU (known under reference ATU1) is located upstream the N gene of the MV. Another ATU (known under reference ATU2) is located between the P and M genes of the MV. Another ATU (known under reference ATU3) is located between the H and L genes of MV. It has been observed that the transcription of the viral RNA of MV follows a gradient from the 5′ to the 3′ end. This explains that, depending on where the heterologous polynucleotide is inserted, its level of expression will vary and be more or less efficient if inserted within ATU1, ATU2 or ATU3.

The first heterologous polynucleotide of the invention is operatively linked, in particular cloned within an ATU inserted within the MV cDNA molecule, preferably an ATU localized between the P and M genes of the MV cDNA molecule, i.e. an ATU2 inserted between the P and M genes of the MV cDNA molecule.

The second heterologous polynucleotide of the invention is operatively linked, in particular cloned within another ATU inserted within the MV cDNA molecule at a location distinct from the location of the first linked, in particular cloned heterologous polynucleotide, preferably an ATU localized between the H and L genes of the MV cDNA molecule, i.e. an ATU3 inserted between the H and L genes of the MV cDNA molecule.

In a particular embodiment of the invention, the third heterologous polynucleotide of the invention is directly fused or indirectly fused to the first heterologous polynucleotide, and the obtained fused heterologous polynucleotide is operatively linked, in particular cloned within an ATU inserted within the MV cDNA molecule, preferably an ATU localized between the P and M genes of the MV cDNA molecule, i.e. an ATU2 inserted between the P and M genes of the MV cDNA molecule.

In a preferred embodiment of the invention, the nucleic acid construct comprises the following polynucleotides encoding polypeptides from 5′ to 3′:
(a) a polynucleotide encoding the N protein of the MV;
(b) a polynucleotide encoding the P protein of the MV;
(c) the first heterologous polynucleotide encoding at least the CS protein of the Plasmodium parasite or the antigenic fragment thereof;
(d) a polynucleotide encoding the M protein of the MV;
(e) a polynucleotide encoding the F protein of the MV;
(f) a polynucleotide encoding the H protein of the MV;
(g) the second heterologous polynucleotide encoding the at least a chimeric antigen of the Plasmodium parasite; and
(h) a polynucleotide encoding the L protein of the MV;
   wherein said polynucleotides are operatively linked, in particular cloned in the nucleic acid construct and under a control of viral replication and transcription regulatory sequences such as MV leader and trailer sequences.

In another preferred embodiment of the invention, the nucleic acid construct comprises the following polynucleotides encoding polypeptides from 5′ to 3′:
(a) a polynucleotide encoding the N protein of the MV;
(b) a polynucleotide encoding the P protein of the MV;
(c) the first heterologous polynucleotide encoding at least the CS protein of the Plasmodium parasite or the antigenic fragment thereof;
(d) the third heterologous polynucleotide encoding at least the RH5 of the Plasmodium parasite or the antigenic fragment thereof, which is directly fused or indirectly fused to the first heterologous polynucleotide of (c);
(e) a polynucleotide encoding the M protein of the MV;
(f) a polynucleotide encoding the F protein of the MV;
(g) a polynucleotide encoding the H protein of the MV;
(h) the second heterologous polynucleotide encoding the at least a chimeric antigen of the Plasmodium parasite; and
(i) a polynucleotide encoding the L protein of the MV;
   wherein said polynucleotides are operatively linked, in particular cloned in the nucleic acid construct and under a control of viral replication and transcription regulatory sequences such as MV leader and trailer sequences.

The expressions “N protein”, “P protein”, “M protein”, “F protein” , “H protein” and “L protein” refer respectively to the nucleoprotein (N), the phosphoprotein (P), the matrix protein (M), the fusion protein (F), the hemagglutinin protein (H) and the RNA polymerase large protein (L) of a MV. These components have been identified in the prior art and are especially disclosed in Fields, Virology (Knipe & Howley, 2001).

In a preferred embodiment of the invention, the measles virus is an attenuated virus strain.

An “attenuated strain” of measles virus is defined as a strain that is avirulent or less virulent than the parent strain in the same host, while maintaining immunogenicity and possibly adjuvanticity when administered in a host i.e., preserving immunodominant T and B cell epitopes and possibly the adjuvanticity such as the induction of T cell costimulatory proteins or the cytokine IL-12.

An attenuated strain of a MV accordingly refers to a strain which has been serially passaged on selected cells and, possibly, adapted to other cells to produce seed strains suitable for the preparation of vaccine strains, harboring a stable genome which would not allow reversion to pathogenicity nor integration in host chromosomes. As a particular “attenuated strain”, an approved strain for a vaccine is an attenuated strain suitable for the invention when it meets the criteria defined by the FDA (US Food and Drug Administration) i.e., it meets safety, efficacy, quality and reproducibility criteria, after rigorous reviews of laboratory and clinical data (www.fda.gov/cber/vaccine/vacappr.htm).

Particular attenuated strains that can be used to implement the present invention and especially to derive the MV cDNA of the nucleic acid construct are the Schwarz strain, the Zagreb strain, the AIK-C strain and the Moraten strain. All these strains have been described in the prior art and access to them is provided in particular as commercial vaccines.

In a particular embodiment of the invention, the cDNA molecule is placed under the control of heterologous expression control sequences. The insertion of such a control for the expression of the cDNA, is favorable when the expression of this cDNA is sought in cell types which do not enable full transcription of the cDNA with its native control sequences.

In a particular embodiment of the invention, the heterologous expression control sequence comprises the T7 promoter and T7 terminator sequences. These sequences are respectively located 5′ and 3′ of the coding sequence for the full length antigenomic (+)RNA strand of MV and from the adjacent sequences around this coding sequence.

In a particular embodiment of the invention, the cDNA molecule, which is defined here above is modified i.e., comprises additional nucleotide sequences or motifs.

In a preferred embodiment, the cDNA molecule of the invention further comprises, at its 5′-end, adjacent to the first nucleotide of the nucleotide sequence encoding the full-length antigenomic (+)RNA strand of the MV approved vaccine strain, a GGG motif followed by a hammerhead ribozyme sequence and which comprises, at its 3′-end, adjacent to the last nucleotide of said nucleotide sequence encoding the full length anti-genomic (+)RNA strand, the sequence of a ribozyme. The Hepatitis delta virus ribozyme (δ) is appropriate to carry out the invention.

The GGG motif placed at the 5′ end, adjacent to the first nucleotide of the above coding sequence improves the efficiency of the transcription of said cDNA coding sequence. As a requirement for the proper assembly of measles virus particles is the fact that the cDNA encoding the antigenomic (+)RNA of the nucleic acid construct of the invention complies with the rule of six, when the GGG motif is added, a ribozyme is also added at the 5′ end of the coding sequence of the cDNA, 3′ from the GGG motif, in order to enable cleavage of the transcript at the first coding nucleotide of the full-length antigenomic (+)RNA strand of MV.

In a particular embodiment of the invention, in order to prepare the nucleic acid construct of the invention, the preparation of a cDNA molecule encoding the full-length antigenomic (+) RNA of a MV disclosed in the prior art is achieved by known methods. Said cDNA provides especially the genome vector when it is inserted in a vector such as a plasmid.

A particular cDNA molecule suitable for the preparation of the nucleic acid construct of the invention is the one obtained using the Schwarz strain of MV. Accordingly, the cDNA used within the present invention may be obtained as disclosed in WO2004/000876 or may be obtained from plasmid pTM-MVSchw deposited by Institut Pasteur at the Collection Nationale de Culture de Microorganismes (CNCM), 28 rue du Dr Roux, 75724 Paris Cedex 15, France, under No 1-2889 on Jun. 12, 2002, the sequence of which is disclosed in WO2004/000876 incorporated herein by reference. The plasmid pTM-MVSchw has been obtained from a Bluescript plasmid and comprises the polynucleotide coding for the full-length measles virus (+) RNA strand of the Schwarz strain placed under the control of the promoter of the T7 RNA polymerase. It has 18967 nucleotides and a sequence represented as SEQ ID NO: 48. cDNA molecules (also designated cDNA of the measles virus or MV cDNA for convenience) from other MV strains may be similarly obtained starting from the nucleic acid purified from viral particles of attenuated MV such as those described herein.

The cDNA used within the present invention may also be obtained from plasmid pTM2-MVSchw-gfp deposited by Institut Pasteur at the Collection Nationale de Culture de Microorganismes (CNCM), 28 rue du Dr Roux, 75724 Paris Cedex 15, France, under No 1-2890 on Jun. 12, 2002. It has 19795 nucleotides and a sequence represented as SEQ ID NO: 49. This plasmid contains the sequence encoding the eGFP marker that may be deleted or substituted.

The nucleic acid construct of the invention is suitable and intended for the preparation of recombinant infectious replicative measles—Malaria virus and accordingly said nucleic acid construct is intended for insertion in a transfer genome vector that as a result comprises the cDNA molecule of the measles virus, especially of the Schwarz strain, for the production of said MV-Malaria virus and yield of at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof. The pTM-MVSchw plasmid or the pTM2-MVSchw plasmid is suitable to prepare the transfer vector, by insertion of the Malaria polynucleotide(s) necessary for the expression of at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof. The recombinant infectious replicating MV-Malaria virus particles may be recovered from rescue helper cells or in production cells. No Virus Like Particles (VLPs) are synthetized since no viral proteins allowing their production are expressed. Malaria antigens are either expressed in free form or in combination with Measles virus particles when said antigens are “anchored” for antigens comprising a transmembrane sequence (i.e. for CSP).

The invention thus relates to a transfer vector, which is used for the preparation of recombinant MV-Malaria virus particles when rescued from helper cells. Advantageously, the transfer vector of the invention is a transfer vector plasmid suitable for transfection of said helper cells or of production cells, comprising the nucleic acid construct of the invention, in particular is a plasmid obtained from a Bluescript plasmid, such as pMV-Malaria.

In a particular embodiment of the invention, the transfer vector plasmid has the sequence of SEQ ID NO: 54 or SEQ ID NO: 55.

The invention also concerns the use of said transfer vector to transform cells suitable for rescue of MV-Malaria virus particles, in particular to transfect or to transduce such cells respectively with plasmids or with viral vectors harboring the nucleic acid construct of the invention, said cells being selected for their capacity to express required MV proteins for appropriate replication, transcription and encapsidation of the recombinant genome of the virus corresponding to the nucleic acid construct of the invention in recombinant infectious replicating MV-Malaria virus particles.

In a preferred embodiment, the invention relates to transformed cells comprising inserted in their genome the nucleic acid construct according to the invention or comprising the transfer vector plasmid according to the invention, wherein said cells are in particular eukaryotic cells, such as avian cells, in particular CEF cells, mammalian cells such as HEK293 cells or yeast cells.

Polynucleotides are thus present in said cells, which encode proteins that include in particular the N, P and L proteins of a MV (i.e., native MV proteins or functional variants thereof), preferably as stably expressed proteins at least for the N and P proteins functional in the transcription and replication of the recombinant MV-Malaria virus particles. The N and P proteins may be expressed in the cells from a plasmid comprising their coding sequences or may be expressed from a DNA molecule inserted in the genome of the cell.

The L protein may be expressed from a different plasmid. It may be expressed transitory. The helper cell is also capable of expressing a RNA polymerase suitable to enable the synthesis of the recombinant RNA derived from the nucleic acid construct of the invention, possibly as a stably expressed RNA polymerase. The RNA polymerase may be the T7 phage polymerase or its nuclear form (nIsT7).

In an embodiment of the invention, the cDNA clone of MV is from the same MV strain as the N protein and/or the P protein and/or the L protein. In another embodiment of the invention, the cDNA clone of a MV is from a different strain of virus than the N protein and/or the P protein and/or the L protein.

The invention also relates to a process for the preparation of recombinant infectious measles virus (MV) particles expressing at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof comprising:
1) transferring, in particular transfecting, the nucleic acid construct of the invention or the transfer vector containing such nucleic acid construct in a helper cell line which also expresses proteins necessary for transcription, replication and encapsidation of the antigenomic (+)RNA sequence of MV from its cDNA and under conditions enabling viral particles assembly; and
2) recovering the recombinant infectious MV-Malaria virus particles expressing at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof. In a particular embodiment of the invention, this process comprises:
1) transfecting helper cells with a nucleic acid construct according to the invention and with a transfer vector, wherein said helper cells are capable of expressing helper functions to express an RNA polymerase, and to express the N, P and L proteins of a MV virus ;
2) co-cultivating said transfected helper cells of step 1) with passaged cells suitable for the passage of the MV attenuated strain from which the cDNA originates ;
3) recovering the recombinant infectious MV-Malaria virus particles expressing at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof.

In another particular embodiment of the invention, the method for the production of recombinant infectious MV-Malaria virus particles expressing at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof comprises:
1) recombining a cell or a culture of cells stably producing a RNA polymerase, the N protein of a MV and the P protein of a MV, with a nucleic acid construct of the invention and with a vector comprising a nucleic acid encoding the L protein of a MV, and
2) recovering the recombinant infectious MV-Malaria virus particles expressing at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof from said recombinant cell or culture of recombinant cells.

In a particular embodiment of said process, recombinant MV are produced, which express at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof.

Preferably, the invention relates to a process to rescue recombinant infectious replicating measles virus (MV)-malaria virus particles expressing (i) at least the circumsporozoite (CS) protein of a Plasmodium parasite or an antigenic fragment thereof, and at least a chimeric antigen of a Plasmodium parasite, or (ii) at least the CS protein of a Plasmodium parasite or an antigenic fragment thereof, at least a chimeric antigen of a Plasmodium parasite and at least the reticulocyte-binding protein homologue 5 (RH5) of a Plasmodium parasite or an antigenic fragment thereof,

wherein said chimeric antigen comprises or consists of the following fragments of (a), (b), (c) and (d) assembled in a fusion polypeptide, wherein the fragments of (a), (b), (c) and (d) elicit a human leukocyte antigen (HLA)-restricted CD8⁺ and/or CD4⁺ T cell response against a Plasmodium parasite, and are directly or indirectly fused in this order:
(a) a fragment of the inhibitor of cysteine protease (ICP) (18-10) of a Plasmodium parasite,
(b) a fragment of the protein Ag45 (11-10) of a Plasmodium parasite,
(c) a fragment of the thrombospondin related anonymous protein (TRAP) of a Plasmodium parasite, and
(d) the protein Ag40 (11-09) of a Plasmodium parasite or a fragment thereof,
   or a chimeric antigen variant thereof, which consists of a chimeric antigen having an amino acid sequence which has at least 90% sequence identity or more than 95% sequence identity or 99% sequence identity with the sequence of the fusion polypeptide consisting of fused fragments of (a), (b), (c) and (d), from which it derives by point mutation of one or more amino acid residues, over its whole length,

and wherein said process comprises:
1) co-transfecting helper cells, in particular HEK293 helper cells, that stably express T7 RNA polymerase, and measles N and P proteins with (i) the transfer vector plasmid according to claim 18 or 19 and with (ii) a vector, especially a plasmid, encoding the MV L polymerase;
2) cultivating said co-transfected helper cells in conditions enabling the production of recombinant MV-malaria virus particles;
3) propagating the thus produced recombinant MV-malaria virus particles by co-cultivating said helper cells of step 2) with cells enabling said propagation such as Vero cells;
4) recovering recombinant infectious replicating MV-malaria virus particles expressing (i) at least the CS protein of the Plasmodium parasite or the antigenic fragment thereof, and said chimeric antigen of the Plasmodium parasite, or (ii) at least the CS protein of the Plasmodium parasite or the antigenic fragment thereof, said chimeric antigen of the Plasmodium parasite and the RH5 of the Plasmodium parasite.

According to a particular embodiment of said process, the transfer vector plasmid has the sequence of SEQ ID NO: 54 or SEQ ID NO: 55.

As used herein, the term “recombining” means introducing at least one polynucleotide into a cell, for example under the form of a vector, said polynucleotide integrating (entirely or partially) or not integrating into the cell genome (such as defined above).

According to a particular embodiment, recombination can be obtained with a first polynucleotide, which is the nucleic acid construct of the invention. Recombination can, also or alternatively, encompasses introducing a polynucleotide, which is a vector encoding a RNA polymerase large protein (L) of a MV, whose definition, nature and stability of expression has been described herein.

In accordance with the invention, the cell or cell lines or a culture of cells stably producing a RNA polymerase, a nucleoprotein (N) of a measles virus and a polymerase cofactor phosphoprotein (P) of a measles virus is a cell or cell line as defined in the present specification or a culture of cells as defined in the present specification, i.e., are also recombinant cells to the extent that they have been modified by the introduction of one or more polynucleotides as defined above. In a particular embodiment of the invention, the cell or cell line or culture of cells, stably producing the RNA polymerase, the N and P proteins, does not produce the L protein of a measles virus or does not stably produce the L protein of a measles virus, e.g., enabling its transitory expression or production.

The production of recombinant infectious replicating MV-Malaria virus particles of the invention may involve a transfer of cells transformed as described herein. The term “transfer” as used herein refers to the plating of the recombinant cells onto a different type of cells, and particularly onto monolayers of a different type of cells. These latter cells are competent to sustain both the replication and the production of infectious MV-Malaria virus particles, i.e., respectively the formation of infectious viruses inside the cell and possibly the release of these infectious viruses outside of the cells. This transfer results in the co-culture of the recombinant cells of the invention with competent cells as defined in the previous sentence. The above transfer may be an additional, i.e., optional, step when the recombinant cells are not efficient virus-producing culture, i.e., when infectious MV-Malaria virus particles cannot be efficiently recovered from these recombinant cells. This step is introduced after further recombination of the recombinant cells of the invention with nucleic acid construct of the invention, and optionally a vector comprising a nucleic acid encoding a RNA polymerase large protein (L) of a measles virus.

In a particular embodiment of the invention, a transfer step is required since the recombinant cells, usually chosen for their capacity to be easily recombined are not efficient enough in the sustaining and production of recombinant infectious MV-Malaria virus particles. In said embodiment, the cell or cell line or culture of cells of step 1) of the above-defined methods is a recombinant cell or cell line or culture of recombinant cells according to the invention.

Cells suitable for the preparation of the recombinant cells of the invention are prokaryotic or eukaryotic cells, particularly animal or plant cells, and more particularly mammalian cells such as human cells or non-human mammalian cells or avian cells or yeast cells. In a particular embodiment, cells, before recombination of its genome, are isolated from either a primary culture or a cell line. Cells of the invention may be dividing or non-dividing cells.

According to a preferred embodiment, helper cells are derived from human embryonic kidney cell line 293, which cell line 293 is deposited with the ATCC under No. CRL-1573. Particular cell line 293 is the cell line disclosed in the international application WO2008/078198 and referred to in the following examples.

According to another aspect of this process, the cells suitable for passage are CEF cells. CEF cells can be prepared from fertilized chicken eggs as obtained from EARL Morizeau, 8 rue Moulin, 28190 Dangers, France, or from any other producer of fertilized chicken eggs.

The process which is disclosed according to the present invention is used advantageously for the production of infectious replicative MV-Malaria virus particles appropriate for use as immunization compositions.

The invention thus relates to an immunogenic composition whose active principle comprises infectious replicative MV-Malaria virus particles rescued from the nucleic acid construct of the invention and in particular obtained by the process disclosed.

The nucleic acid construct of the invention and the MV-Malaria of the invention encode or express at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof.

According to a preferred embodiment, the invention also concerns modifications and optimization of the polynucleotide to allow an efficient expression of at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof, at the surface of chimeric infectious particles of MV-Malaria in the host, in particular the human host.

According to this embodiment, optimization of the polynucleotide sequence can be operated avoiding cis-active domains of nucleic acid molecules: internal TATA-boxes, chi-sites and ribosomal entry sites; AT-rich or GC-rich sequence stretches; ARE, INS, CRS sequence elements; repeat sequences and RNA secondary structures ; cryptic splice donor and acceptor sites, branch points.

The optimized polynucleotides may also be codon optimized for expression in a specific cell type. This optimization allows increasing the efficiency of chimeric infectious particles production in cells without impacting the expressed protein(s).

In a particular embodiment of the invention, the first, second and third heterologous polynucleotides as defined above have been optimized for a Macaca codon usage or have been optimized for a human codon usage.

The optimization of the polynucleotides encoding at least the CS of the Plasmodium parasite or the antigenic fragment thereof, at least the above-defined chimeric antigen of the Plasmodium parasite and optionally at least the RH5 of the Plasmodium parasite or the antigenic fragment thereof may be performed by modification of the wobble position in codons without impacting the identity of the amino acid residue translated from said codon with respect to the original one.

Optimization is also performed to avoid editing-like sequences from Measles virus. The editing of transcript of Measles virus is a process which occurs in particular in the transcript encoded by the P gene of Measles virus. This editing, by the insertion of extra G residues at a specific site within the P transcript, gives rise to a new protein truncated compared to the P protein. Addition of only a single G residue results in the expression of the V protein, which contains a unique carboxyl terminus (Cattaneo R et al., Cell. 1989 Mar. 10; 56(5):759-64).

In a particular embodiment of the invention, measles editing-like sequences have been deleted from said first, second and third heterologous polynucleotides. The following measles editing-like sequences can be mutated: AAAGGG, AAAAGG, GGGAAA, GGGGAA, TTAAA, AAAA, as well as their complementary sequence: TTCCCC, TTTCCC, CCTTTT, CCCCTT, TTTAA, TTTT. For example, AAAGGG can be mutated in AAAGGC, AAAAGG can be mutated in AGAAGG or in TAAAGG or in GAAAGG, and GGGAAA in GCGAAA.

In a particular embodiment of the invention, the native and codon-optimized nucleotide sequences of the polynucleotide encoding particular peptides/proteins/antigen as well as the amino acid sequences of these peptides/proteins/antigen of the invention are the sequences disclosed as SEQ ID NOs: 1-47 and 56-59 and mentioned in Table 1 below.

In a particular embodiment of the invention, the transfer vector plasmid pTM2-MVSchw_CSPf has the sequence of SEQ ID NO: 50, the transfer vector plasmid pTM2-MVSchw_CSPb has the sequence of SEQ ID NO: 51, the transfer vector plasmid pTM2-MVSchw_RH5-TM-CSPf-TM (i. e. pTM2-FaIVAX-TM) has the sequence of SEQ ID NO: 52, the transfer vector plasmid pTM2-MVSchw_RH5-CSPf (i.e. pTM2-FaIVAX-Sol) has the sequence of SEQ ID NO: 53, the transfer vector plasmid pTM2-MVSchw_CSPb-3-PbFus with the signal peptide from the F protein of MV Schwarz has the sequence of SEQ ID NO: 54 and the transfer vector plasmid pTM2-MVSchw_CSPb-3-PbFus without the signal peptide from the F protein of MV Schwarz has the sequence of SEQ ID NO: 55, as mentioned in Table 1 below.

TABLE 1
Native and codon-optimized nucleotide sequences of the polynucleotide
encoding particular peptides/proteins as well as amino acid sequences
of these peptides/proteins used in the invention.

| | | SEQ ID NO of | |
|---|---|---|---|
| | SEQ ID NO of | the codon- | |
| | the native | optimized | |
| | nucleotide | (CO) nucleotide | SEQ ID NO of |
| | sequence of the | sequence of the | the amino |
| Name of the compound, i.e. | polynucleotide | polynucleotide | acid sequence |
| peptide/protein/antigen | encoding the | encoding the | of the |
| (abbreviation) | compound | compound | compound |
| Signal peptide from the F | | 1 | 2 |
| protein of MV Schwarz | | | |
| Intracytoplasmic and | | 3 | 4 |
| transmembrane domains | | | |
| of the F protein of | | | |
| MV Schwarz | | | |
| Intergenic sequence | | 5 | 6 |
| ATU | | 7 | |
| CSPb from from | | 8 | 9 |
| P. berghei ANKA | | (mouse CO) | |
| CSPf from | | 10 | 11 |
| P. falciparum 3D7 | | (human CO) | |
| CSPf-TM from | | 12 | 13 |
| P. falciparum 3D7 | | (human CO) | |
| First fragment of the | | 14 | 15 |
| inhibitor of cysteine | | (mouse CO) | |
| protease (ICP) from | | | |
| P. berghei ANKA | | | |
| (Pb18-10 NT = Pb18-10 | | | |
| N-terminal) | | | |
| Second fragment of the | | 16 | 17 |
| inhibitor of cysteine | | (mouse CO) | |
| protease (ICP) from | | | |
| P. berghei ANKA | | | |
| (Pb18-10CT = Pb18-10 | | | |
| C-terminal) | | | |
| Fragment of the inhibitor | | 18 | 19 |
| of cysteine protease (ICP) | | (human CO) | |
| from P. falciparum 3D7 | | | |
| (Pf18-10-SP = Pf18-10 | | | |
| devoid of its signal peptide) | | | |
| Fragment of the protein | | 20 | 21 |
| Ag45 (11-10) from | | (mouse CO) | |
| P. berghei ANKA | | | |
| (Pb11-10CT = Pb11-10 | | | |
| C-terminal) | | | |
| Fragment of the protein | | 22 | 23 |
| Ag45 (11-10) from | | (human CO) | |
| P. falciparum 3D7 | | | |
| (Pf11-10CT) | | | |
| Fragment of the thrombospondin | | 24 | 25 |
| related anonymous protein (TRAP) | | (mouse CO) | |
| from P. berghei ANKA | | | |
| (Pb TRAP NT = Pb TRAP | | | |
| N-terminal) | | | |
| Fragment of the thrombospondin | | 26 | 27 |
| related anonymous protein (TRAP) | | (human CO) | |
| from P. falciparum 3D7 | | | |
| (Pf TRAP NT = Pf TRAP | | | |
| N-terminal) | | | |
| protein Ag40 (11-09) from | | 28 | 29 |
| P. berghei ANKA | | (mouse CO) | |
| (Pb11-09) | | | |
| protein Ag40 (11-09) from | | 30 | 31 |
| P. falciparum 3D7 | | (human CO) | |
| (Pf11-09) | | | |
| RH5 | 32 | | 33 |
| PS-f-RH5- | | 58 | 59 |
| mut N-glyco | | (human CO) | |
| PS-f-RH5-TM | 34 | | 35 |
| Insert RH5-CSPf | | 36 | 37 |
| PS-f-RH5-TM- | | 56 | 57 |
| mut N-glyco | | (human CO) | |
| Insert RH5-TM-CSPf-TM | | 38 | 39 |
| Pb Fusion from | | 40 | 41 |
| P. berghei ANKA, | | (mouse CO) | |
| with the signal peptide from | | | |
| the F protein of MV Schwarz | | | |
| Pb Fusion from | | 42 | 43 |
| P. berghei ANKA, | | (mouse CO) | |
| without the signal peptide from | | | |
| the F protein of MV Schwarz | | | |
| Pf Fusion from | | 44 | 45 |
| P. falciparum 3D7, | | (human CO) | |
| with the signal peptide from | | | |
| the F protein of MV Schwarz | | | |
| Pf Fusion from | | 46 | 47 |
| P. falciparum 3D7, | | (human CO) | |
| without the signal peptide from | | | |
| the F protein of MV Schwarz | | | |

| Name of the transfer vector plasmid | SEQ ID NO |
|---|---|
| pTM-MVSchw | 48 |
| pTM2-MVSchw-gfp | 49 |
| pTM2-MVSchw_CSPf | 50 |
| pTM2-MVSchw_CSPb | 51 |
| pTM2-MVSchw_RH5-TM-CSPf-TM | 52 |
| (i.e. pTM2-FalVAX-TM) | |
| pTM2-MVSchw_RH5-CSPf | 53 |
| (i.e. pTM2-FalVAX-Sol) | |
| pTM2-MVSchw_CSPb-3-PbFus with | 54 |
| the signal peptide from the | |
| F protein of MV Schwarz | |
| pTM2-MVSchw_CSPb-3-PbFus without | 55 |
| the signal peptide from the | |
| F protein of MV Schwarz | |

It should be noted that any amino acid sequence disclosed therein may further comprise a 5′ end extra-methionine. It should also be noted that any nucleotide sequence disclosed therein may further comprise additional nucleotides towards the 5′ end of the sequence, said additional nucleotides comprising a start codon (i.e. an atg codon), in particular when no atg codon is present in the main ORF. Furthermore, it should also be noted that any construct, in particular a construct comprising RH5, may also be mutated on (predictive) N-glycosylation site(s). As an example, a threonine residue may be substituted for an alanine residue by mutating the codon encoding the threonine localized on a (predictive) N-glycosylation site. As another example, SEQ ID NO: 57 and SEQ ID NO: 59 correspond to RH5 with mutated N-glycosylation sites, as compared with SEQ ID NO: 33 and SEQ ID NO: 35 which correspond to RH5 without mutation on their N-glycosylation site. Mutations within the polynucleotides encoding N-glycosylated NH5 are represented in SEQ ID NO: 56 and in SEQ ID NO: 58, and may be compared with polynucleotides encoding counterpart non-mutated-N-glycosylation-site NH5, respectively SEQ ID NO: 34 and SEQ ID NO: 32.

In a particular embodiment of the invention, the Plasmodium parasite is selected from the group consisting of Plasmodium falciparum, Plasmodium malariae, Plasmodium vivax, Plasmodium ovale, Plasmodium knowlesi and Plasmodium berghei, preferably is Plasmodium falciparum or Plasmodium berghei.

In a particular embodiment of the invention, the Plasmodium parasite is Plasmodium falciparum and said first heterologous polynucleotide encoding at least the CS protein of Plasmodium falciparum or the antigenic fragment thereof further encodes (i) the signal peptide from the F protein of the MV or (ii) the signal peptide from the F protein of the MV and the intracytoplasmic and transmembrane domains of the F protein of the MV.

In a particular embodiment of the invention, the second heterologous polynucleotide encoding the at least a chimeric antigen of the Plasmodium parasite further encodes (i) the signal peptide from the F protein of the MV.

In a particular embodiment of the invention, the third heterologous polynucleotide encoding at least the RH5 of the Plasmodium parasite or the antigenic fragment thereof further encodes (i) the signal peptide from the F protein of the MV or (ii) the signal peptide from the F protein of the MV and the signal peptide from the F protein of the MV and the intracytoplasmic and transmembrane domains of the F protein of the MV.

In a particular embodiment of the invention, the codon-optimized nucleotide sequences of the polynucleotide encoding the CS protein of a Plasmodium parasite are selected from the group consisting of SEQ ID NO: 8, SEQ ID NO: 10 and SEQ ID NO: 12, and the amino acid sequences of the CS protein of a Plasmodium parasite are the sequences disclosed as SEQ ID NO: 9, SEQ ID NO: 11 and SEQ ID NO: 13.

An antigenic fragment of the CS protein of a Plasmodium parasite may be used in the present invention.

In a particular embodiment of the invention, the antigenic fragment of the CS protein of a Plasmodium parasite is a truncated version of the CS protein, in particular a truncated form from 19 to 369 amino acids.

In a preferred embodiment of the invention, the antigenic fragment of the CS protein of a Plasmodium parasite is a truncated form devoid of the GPI anchored signal at the C-terminus. Said GPI anchored signal may correspond to the last 12 amino acid residues in the C-terminal part in the native amino acid sequence of the CS protein.

In a more preferred embodiment of the invention, said antigenic fragment of the CS protein of a Plasmodium parasite further comprises (i) the signal peptide from the F protein of the MV at N-terminus, e.g. MV Schwarz, and/or (ii) the intracytoplasmic and transmembrane domains of the F protein of a MV, e.g. MV Schwarz.

In a preferred embodiment of the invention, in order to improve vaccine efficacy, other antigens such as ICP, Ag45, TRAP, Ag40, RH5 or the antigenic fragment of ICP, Ag45, TRAP, Ag40, RH5 are added to the CS protein of a Plasmodium parasite or the antigenic fragment of the CS protein of a Plasmodium parasite.

In a preferred embodiment of the invention, in the chimeric antigen, the fragment of the ICP (18-10) of the Plasmodium parasite of (a) has the amino acid sequence of SEQ ID NO: 14, SEQ ID NO: 16 and SEQ ID NO: 18, the fragment of the protein Ag45 (11-10) of the Plasmodium parasite of (b) has the amino acid sequence of SEQ ID NO: 20 or SEQ ID NO: 22, the fragment of the TRAP of the Plasmodium parasite of (c) has the amino acid sequence of SEQ ID NO: 24 or SEQ ID NO: 26, and the protein Ag40 (11-09) of the Plasmodium parasite or the fragment thereof of (d) has the amino acid sequence of SEQ ID NO: 28 or SEQ ID NO: 30.

The inhibitor of cysteine protease ICP (18-10) refers to a protein that seems to be involved in the motility and infectivity capacity of sporozoites via the processing of CSP. The Pb18-10 N-terminal (NT) and C-terminal (CT) Protective domains (PDs) comprise the sequence of amino acids from 24 to 139 and from 189 to 355, respectively, of the mouse-codon optimized Pb 18-10 (SEQ ID NOs: 15, 17). Said Pb 18-10 NT and CT PDs may (i) be devoid of signal peptide sequence and (ii) further comprise CD8+ T cell epitopes predicted to be good binders to rodent MHC class I molecules. Pf 18-10 PD comprises the sequence of amino acids from 24 to 414 of the human-codon optimized Pf 18-10 (SEQ ID NO: 19). Said Pf 18-10 PDs may (i) be devoid of signal peptide sequence and (ii) further comprise T cell epitopes predicted to be good binders to human MHC class I or II molecules.

The fragment of the protein Ag45 (11-10) of a Plasmodium parasite of (b) refers to a protein that has been called 11-10 (SEQ ID NO: 21, 23). This protein doesn't have annotated domains, but possesses a central region with negatively charged amino acids. Recently the 11-10 ortholog of Plasmodium yoelii, another rodent-infecting plasmodial species, was also identified as a protective antigen (Boysen et al. MBio 2013, 4(6):e00874-13). The deletion of the gene coding for the antigen 11-10 blocked the Pb sporozoite invasion of salivary glands and completely abolished the capacity of sporozoites to infect the liver. The Pb 11-10 CT PD comprises the sequence of amino acids from 186 to 352 of the mouse-codon optimized Pb 11-10 (SEQ ID NO: 21). Said Pb 11-10 CT PD may (i) be devoid of signal peptide sequence and (ii) further comprise CD8+ T cell epitopes predicted to be good binders to rodent MHC class I molecules. The Pf 11-10 CT PD comprises the sequence of amino acids from 217 to 395 of the human-codon optimized Pf 11-10 (SEQ ID NO: 23). Said Pf 11-10 CT PD may (i) be devoid of signal peptide sequence and (ii) further comprise T cell epitopes predicted to be good binders to human MHC class I or II molecules.

The fragment of the TRAP of a Plasmodium parasite of (c) (thrombospondin related anonymous protein; 11-05) refers to a type I transmembrane protein harboring two extracellular adhesive domains, a von Willebrand factor type A domain and a thrombospondin type 1 domain, followed by a proline-rich repetitive region. TRAP is stored in micronemal secretory vesicles and following parasite activation, the protein is translocated to the surface of sporozoites where it serves as a linker between a solid substrate and the cytoplasmic motor of sporozoites. Intriguingly, anti-TRAP antibodies do not impair parasite motility and infectivity CD8+ T cells seem to mediate the protection mediated by TRAP immunization. The PbTRAP NT PD comprises the sequence of amino acids from 24 to 263 of the mouse-codon optimized Pb TRAP (SEQ ID NO: 25). Said Pb TRAP NT PD may (i) be devoid of signal peptide sequence and (ii) further comprise CD8+ T cell epitopes predicted to be good binders to rodent MHC class I molecules. The PfTRAP NT PD comprises the sequence of amino acids from 28 to 320 of the human-codon optimized Pf TRAP (SEQ ID NO: 27). Said Pf TRAP NT PD may (i) be devoid of signal peptide sequence and (ii) further comprise T cell epitopes predicted to be good binders to human MHC class I or II molecules.

The protein Ag40 (11-09) of a Plasmodium parasite of (d) refers to a hypothetical protein that has been called 11-09. This protein has 4-5 annotated transmembrane domains. Deletion of the gene coding for the antigen 11-09 caused impairment of Pb parasite development in the liver. The Pb 11-09 PD comprises the sequence of amino acids from 3 to 211 of the mouse-codon optimized Pb 11-09 (SEQ ID NO: 29). Said Pb 11-09 PD may (i) comprise CD8+ T cell epitopes predicted to be good binders to rodent MHC class I molecules. Said Pf 11-09 PD comprises the sequence of amino acids from 7 to 211 of the human-codon optimized Pf 11-09 (SEQ ID NO: 31). Said Pf 11-09 PD may (i) comprise T cell epitopes predicted to be good binders to human MHC class I or II molecules.

In a particular embodiment of the invention, the RH5 of the Plasmodium parasite has the sequence of SEQ ID NO: 33 or SEQ ID NO: 35 or SEQ ID NO: 57 or SEQ ID NO: 59.

In a preferred embodiment of the invention, the first heterologous polynucleotide encoding at least the CS protein of the Plasmodium parasite or the antigenic fragment thereof has a sequence selected from the group consisting of SEQ ID NO: 8, SEQ ID NO: 10 and SEQ ID NO: 12, and the second heterologous polynucleotide encoding the at least a chimeric antigen of the Plasmodium parasite has a sequence selected from the group consisting of SEQ ID NO: 40, SEQ ID NO: 42, SEQ ID NO: 44 and SEQ ID NO: 46.

In a preferred embodiment of the invention, the third heterologous polynucleotide has the sequence of SEQ ID NO: 32 or the sequence of SEQ ID NO: 34 or the sequence of SEQ ID NO: 56 or the sequence of SEQ ID NO: 58.

In a preferred embodiment of the invention, the first heterologous polynucleotide encodes the CS protein of the Plasmodium parasite or the antigenic fragment thereof whose sequence is selected from the group consisting of SEQ ID NO: 9, SEQ ID NO: 11 and SEQ ID NO: 13, and the second heterologous polynucleotide encodes the chimeric antigen of the Plasmodium parasite whose sequence is selected from the group consisting of SEQ ID NO: 41, SEQ ID NO: 43, SEQ ID NO: 45 and SEQ ID NO: 47.

In a preferred embodiment of the invention, the third heterologous polynucleotide encodes the RH5 of the Plasmodium parasite or the antigenic fragment thereof whose sequence is SEQ ID NO: 33 or SEQ ID NO: 35 or SEQ ID NO: 57 or SEQ ID NO: 59.

In a particular embodiment of the invention, said nucleic acid construct comprises a first polynucleotide whose sequence is selected from the group consisting of SEQ ID NO: 8x, SEQ ID NO: 10 and SEQ ID NO: 12, and a second polynucleotide whose sequence is selected from the group consisting of SEQ ID NO: 40, SEQ ID NO: 42, SEQ ID NO: 44 and SEQ ID NO: 46.

In another particular embodiment of the invention, said nucleic acid construct comprises a first polynucleotide whose sequence is SEQ ID NO: 36 or SEQ ID NO: 38, and a second polynucleotide whose sequence is selected from the group consisting of SEQ ID NO: 40, SEQ ID NO: 42, SEQ ID NO: 44 and SEQ ID NO: 46.

In a preferred embodiment of the invention, the nucleic acid construct comprises (i) the sequence from nucleotide at position 3532 to nucleotide at position 4558 in the sequence of SEQ ID NO: 54, and the sequence from nucleotide at position 10468 to nucleotide at position 13240 in the sequence of SEQ ID NO: 54, or (ii) the sequence from nucleotide at position 3532 to nucleotide at position 4558 in the sequence of SEQ ID NO: 55, and the sequence from nucleotide at position 10468 to nucleotide at position 13165 in the sequence of SEQ ID NO: 55.

The invention also concerns recombinant infectious replicating measles virus (MV)-malaria virus particles, which comprise as their genome a nucleic acid construct according to the invention.

In a particular embodiment of the invention, said recombinant infectious replicating MV-malaria virus particles are rescued from a helper cell line expressing an RNA polymerase recognized by said cell line, for example a T7 RNA polymerase, a nucleoprotein (N) of a MV, a phosphoprotein (P) of a MV, and optionally an RNA polymerase large protein (L) of a MV, and which helper cell line is further transfected with the transfer vector plasmid according to the invention.

Said recombinant infectious replicating MV-malaria virus particles are thus produced by a method comprising expressing the nucleic acid construct according to the invention in a host cell comprising an RNA polymerase recognized by said host cell, for example a T7 RNA polymerase, a nucleoprotein (N) of a MV, a phosphoprotein (P) of a MV, and optionally an RNA polymerase large protein (L) of a MV.

According to a particular embodiment of the invention, said virus particles comprise in their genome a polynucleotide sequence comprising (i) a first polynucleotide whose sequence is selected from the group consisting of SEQ ID NO: 8, SEQ ID NO: 10 and SEQ ID NO: 12, and a second polynucleotide whose sequence is selected from the group consisting of SEQ ID NO: 40, SEQ ID NO: 42, SEQ ID NO: 44 and SEQ ID NO: 46, or (ii) a first polynucleotide whose sequence is SEQ ID NO: 36 or SEQ ID NO: 38, and a second polynucleotide whose sequence is selected from the group consisting of SEQ ID NO: 40, SEQ ID NO: 42, SEQ ID NO: 44 and SEQ ID NO: 46.

According to another aspect, the invention relates to recombinant infectious MV-malaria virus particles expressing at least the CS protein of the Plasmodium parasite or the antigenic fragment thereof, at least the above-defined chimeric antigen of the Plasmodium parasite and optionally at least the RH5 of the Plasmodium parasite or the antigenic fragment thereof, in particular by reference to their nucleic acid and polypeptide sequences.

According to a preferred embodiment of the invention, the recombinant MV vector is designed in such a way and the production process involves cells such that the virus particles produced in helper cells transfected or transformed with said vector, originated from a MV strain adapted for vaccination, enable the production of recombinant infectious replicating MV for use in immunogenic compositions, preferably protective or even vaccine compositions.

Advantageously, the genome of the recombinant infectious MV-malaria virus particles of the invention is replication competent. By “replication competent’, it is meant a nucleic acid, which when transduced into a helper cell line expressing the N, P and L proteins of a MV, is able to be transcribed and expressed in order to produce new viral particles.

Replication of the recombinant virus of the invention obtained using MV cDNA for the preparation of the recombinant genome of MV-malaria can also be achieved in vivo in the host, in particular the human host to which recombinant MV-malaria is administered.

The invention also relates to a composition or an assembly of immunologically active ingredients comprising the recombinant infectious replicating MV-malaria virus particles according to the invention.

According to a preferred embodiment of the invention, said composition or assembly of immunologically active ingredients is used in the elicitation of a protective immune response against a Plasmodium infection by the elicitation of antibodies directed against said proteins of a Plasmodium parasite, and/or of a cellular immune response, in a host, in particular a human host in need thereof.

Said composition or assembly of immunologically active ingredients accordingly may comprise a suitable vehicle for administration e.g. a pharmaceutically acceptable vehicle to a host, especially a human host and may further comprise but not necessarily adjuvant to enhance immune response in a host. The inventors have indeed shown that the administration of the immunologically active ingredients of the invention may elicit an immune response without the need for adjuvantation.

According to a particular embodiment of the invention, said composition or assembly of immunologically active ingredients comprises a pharmaceutically acceptable vehicle.

The invention relates in particular to a composition, in particular an immunogenic composition, preferably a vaccine composition for administration to children, adolescents or travelers.

In a particular embodiment, said composition or vaccine is used for protection against a Plasmodium infection or against clinical outcomes of infection by a Plasmodium parasite (protection against Malaria) in a prophylactic treatment.

Such a vaccine composition has advantageously immunologically active principles (immunologically active ingredients), which comprise recombinant infectious replicating MV-malaria virus particles rescued from the vector as defined herein, and enabling elicitation of an immune response in a host, in particular a human host.

In the context of the invention, the terms “associated” or “in association” refer to the presence, in a unique composition, of both recombinant infectious replicating MV-malaria virus particles and the above-mentioned proteins of a Plasmodium parasite.

The invention also concerns the recombinant infectious replicating MV-malaria virus particles according to the invention in association with the above-mentioned proteins of a Plasmodium parasite, or the composition or the assembly of immunologically active ingredients according to the invention, for use in the prevention of a Plasmodium infection in a subject or in the prevention of clinical outcomes of infection by a Plasmodium parasite in a subject, in particular in a human.

The invention also concerns the recombinant infectious replicating MV-malaria virus particles according to the invention in association with the above-mentioned proteins of a Plasmodium parasite, for use in an administration scheme and according to a dosage regime that elicit an immune response, advantageously a protective immune response, against a Plasmodium infection or induced disease, in particular in a human host.

The administration scheme and dosage regime may require a unique administration of a selected dose of the recombinant infectious replicating MV-malaria virus particles according to the invention in association with the above-mentioned proteins of a Plasmodium parasite.

Alternatively it may require multiple administration doses, in particular in a prime-boost regimen. Priming and boosting may be achieved with identical immunologically active ingredients consisting of said recombinant infectious replicating MV-malaria virus particles in association with the above-mentioned proteins of a Plasmodium parasite.

Alternatively priming and boosting administration may be achieved with different immunologically active ingredients, involving said recombinant infectious replicating MV-malaria virus particles in association with the above-mentioned proteins of a Plasmodium parasite, in at least one of the administration steps and other active immunogens of Malaria, such as the above-mentioned proteins of a Plasmodium parasite, in other administration steps.

Considering available knowledge on doses of known human MV vaccines, the inventors have determined that the recovery of the recombinant infectious replicating MV-malaria virus particles of the invention enables proposing administration of effective low doses of the active ingredients. A suitable dose of the recombinant infectious replicating MV-malaria virus particles of the invention to be administered may be in the range of 10³ to 10⁶ TCID50, and possibly as low as 10³ to 10⁶ TCID50.

According to a particular embodiment of the invention, the immunogenic or vaccine composition defined herein may also be used for protection against an infection by the measles virus.

The present invention also relates to a method to prevent a Plasmodium infection or clinical outcomes of infection by a Plasmodium parasite, in a subject, in particular in a human subject, comprising administering a pharmaceutically effective quantity of recombinant MV-malaria virus particles according to the invention or an immunogenic composition according to the invention, wherein said particles or composition are in admixture with a pharmaceutically acceptable vehicle; and/or an adjuvant.

As used herein, the term “to prevent” refers to a method by which a Plasmodium infection is obstructed or delayed.

As defined herein, a “pharmaceutically acceptable vehicle” encompasses any substance that enables the formulation of the nucleic acid construct, the vector, in particular the recombinant MV genome vector according to the invention within a composition. A vehicle is any substance or combination of substances physiologically acceptable i.e., appropriate for its use in a composition in contact with a host, especially a human, and thus non-toxic. Examples of such vehicles are phosphate buffered saline solutions, distilled water, emulsions such as oil/water emulsions, various types of wetting agents sterile solutions and the like.

As defined herein, an “adjuvant” includes, for example, liposomes, oily phases, such as Freund type adjuvants, generally used in the form of an emulsion with an aqueous phase or can comprise water-insoluble inorganic salts, such as aluminium hydroxide, zinc sulphate, colloidal iron hydroxide, calcium phosphate or calcium chloride.

Other features and advantages of the invention will be apparent from the examples which follow and will also be illustrated in the figures.

### BRIEF DESCRIPTION OF THE FIGURES

FIG. 1. rMV-CSPb and rMV CSPf. (A) Schematic representation of measles vector expressing CS protein from Plasmodium berghei ANKA (CSPb) or Plasmodium falciparum (CSPf). The synthetic sequence was mammalian codon-optimized and cloned into the Additional Transcription Unit (ATU) position 2 of pTM-Schwarz. The MV genes are indicated as follows: nucleoprotein (N), phosphoprotein (P), V and C accessory proteins, matrix (M), fusion (F), hemagglutinin (H) and polymerase (L). T7 RNA polymerase promoter (T7), T7 RNA polymerase terminator (T7t), hepatitis delta virus ribozyme (∂), hammerhead ribozyme (hh) are requested for viral rescue. (B) Growth curves of MV-Schwarz, rMV-CSPb and rMV-CSPf in Vero cells infected at an MOI of 0.1. Cell-associated virus titers are indicated in TCID50/ml. (C) Detection by western-blot of CSPb or CSPf in cell lysates (L) or supernatant (SN) of Vero cells infected by rMV-CSPb and rMV CSPf. (D) Immunofluorescence detection of CSPb and CSPf in Vero cells infected for 24 hours with rMV-CSPb and rMV-CSPf at an MOI of 0.1. Infected cells formed syncytia where are localized CS proteins.

FIG. 2. Blood parasitemia of C57BL/6 and hCD46IFNAR mice after skin microinjection of 5,000 sporozoites of Plasmodium berghei ANKA. Percentage of infected red blood cells (iRBCs) at day 4, 5 and 6 post-infection (p.i.) was log transformed for parasitemia normalization before statistical analysis. No statistically significant difference was observed between both groups. N.I. threshold of parasitemia detection.

FIG. 3. Immunogenicity and protective efficacy of rMV-CSPb. Antibody response induced in hCD46IFNAR mice immunized with rMV-CSPb at day 0 and 4 weeks later. The data show the reciprocal endpoint dilution titers of specific antibodies to MV (A) and CSPb (B). Percentage of asymptomatic (C) and non-infected (D) hCD46IFNAR mice (6 mice per group) immunized twice at one month of interval and challenged 3 weeks after with 5,000 Pb ANKA sporozoïtes intradermally. (E) Log of parasitemia at day 4, 5 and 6 post-infection (p.i.) (means+/−SD). Asterisks (*) indicate significant mean differences (** for p<0.01; *** for p<0,001). L.D. level of detection. N.I. threshold of parasitemia detection.

FIG. 4. Immunogenicity and protective efficacy of rMV-CSPf. (A-C) Antibody response induced in hCD46IFNAR mice immunized with rMV-CSPf at day 0 and 4 weeks later. Long-term memory was assessed at week 22 post-priming. The data show the reciprocal endpoint dilution titers of specific antibodies to MV (A) and CSPf (B). (C) IgG subtypes of CSPf antibodies elicited by rMV-CSPf 4 weeks after the second immunization. (D-E) Infectious challenge with 5,000 sporozoïtes of a recombinant PbA expressing CSPf repeat sequence 3 weeks (D) or 16 weeks (E) after the second immunization. Log of parasitemia at days 4, 5 and 6 post-infection (p.i.) (means+/−SD). Asterisks (*) indicate significant mean differences (* for p<0.05; ** for p<0,01). L.D. level of detection. N.I. threshold of parasitemia detection.

FIG. 5. Cellular response to rMV-CSPf. (A) IFNγ Elispot assay and (B) intracellular cytokine staining assay were done on freshly extracted splenocytes 7 days after one immunization i.p. with 1.105 TCID50 of MV-Schwarz or rMV-CSPf. Splenocytes were restimulated with inactivated MV-Schwarz at an MOI of 1 or CSPf recombinant LPS-free protein at 50 μg/ml. CD4+ and CD8+ T-cells were stained against IFNγ and TNFα. Negative controls, cultured with media alone, showed less than 0.05% of positive cells (data not shown). Percentile IFNγ and TNFα cytokine distribution for CD4+ and CD8+ T-cells reactive against MV-Schwarz (B1) and upon CSPf restimulation (B2). Asterisks (*) indicate significant mean differences (** for p<0.01).

FIG. 6. Scheme of a chimeric antigen of Plasmodium berghei (P. berghei Fusion 4cPEAg). 4cPEAg refers to the combination of PbTRAP, antigen Pb18-10, antigen Pb11-10 and antigen Pb11-09. Fusion 4cPEAg refers to the chimeric antigen formed by the fusion of the antigen Pb18-10 without its signal peptide (SP), followed by the protective domains 11-10CT and TRAPNT, and the antigen Pb11-09. GPI (glycosylphosphatidylinositol), TSR (thrombospondin type I repeat).

FIG. 7. P. falciparum 4cPEAg fusion. Epitopes from the Pf 4cPEAgs predicted to bind to the Human Leukocyte Antigen (HLA) were identified using the immune epitope database (iedb; www.iedb.org). (a-c) Bars represent epitopes predicted to bind on the HLA-DRB1*01:01, *03:01, *04:01, *04:05, *07:01, *08:02, *09:01, *11:01, *12:01, *13:02 and *15:01. Triangles represent epitopes predicted to bind to the HLA A*01:01, *02:01, *02:03, *02:06, *03:01, *11:01, *23:01,*24:02, *26:01, *30:01, *30:02, *31:01, *32:01, *33:01, *68:01 and *68:02. Inverted triangles represent epitopes predicted to bind to the HLA-B*07:02, *08:01, *15:01, *35:01, *40:01, *44:02, *44:03, *51:01, *53:01, *57:01 and *58:01. White horizontal bars represent the regions used to design the Pf 4cPEAg fusion based on the content of class I and II predicted epitopes and structural/sequence similarity with the protective domains tested using P. berghei. Gray shadows represent conserved structural domains depicted in FIG. 6. SP, signal peptide. Antigens are (a) Pf18-10, (b) Pf11-10, (c) PfTRAP, (d) Pf11-09. (e) Selected regions of the Pf 4cPEAgs (white bars) were chimerized generating the Pf 4cPEAg Fusion. The dotted lines represent the junction between two adjacent antigens/protective domains and show the absence of formation of neo-epitopes.

FIG. 8. Plasmodium berghei infectious challenge in hCD46IFNAR mice immunized with MV Schwarz (G1, control group), MV-CSPb (G2), MV-Pb Fusion (G3), MV-CSPb-Pb Fusion (G4), and MV-CSPb+MV-Pb Fusion (G5). (A) Parasitemia at day 3, 4, 5, and 6 post-challenge. (B) Percentage of non-infected mice after challenge for the five groups (sterile protection). Groups of 6 mice were immunized i.p. (100 μl, 10⁵ TCID₅₀ of each virus, two viruses in two injections for G5) two times at one month interval. The infectious challenge was done with Plasmodium berghei ANKA strain, a model of cerebral malaria, four weeks after the second immunization, with 5,000 sporozoites injected i.d. in footpads. Blood parasitemia was assessed by flow cytometry on a drop of blood from day 3 to 6 post-challenge, until day 10 for sterilely protected mice.

### EXAMPLES

### Material and Methods

### Cell Culture

Vero cells (African green monkey kidney cells) and HEK293-T7-MV (human embryonic kidney cells) helper cells were maintained in Dulbecco's modified Eagle medium (DMEM; Gibco) supplemented with 10% heat-inactivated fetal calf serum (GE Healthcare) and 10.000 U/ml of penicillin-streptomycin (Life technologies). HEK293-T7-MV helpers cells stably expressed T7 polymerase and MV-N and MV-P proteins and were used for measles viral rescue (WO2008/078198).

Construction of pTM2-MVSchw-CSPb and pTM2-MVSchw-CSPf Plasmids and Rescue of rMV-CSPb and rMV-CSPf Recombinant Viruses

The plasmid pTM2-Schw (Combredet, et al. J. Virol. 2003, 77(21):11546-54) encodes the cDNA of the anti-genome of the Schwarz MV vaccine strain with an additional transcription unit (ATU) between the phosphoprotein (MV-P) and the matrix (MV-M) genes, flanked by BsiWI/BssHII restriction sites. Two cDNAs encoding the circumsporozoite protein of Plasmodium berghei ANKA (CSPb ANKA full length sequence, mammalian codon optimized synthetic gene, Eurofins Genomics) and the circumsporozolte protein of Plasmodium falciparum 3D7 (CSPf, truncated form from 19 to 369 aa, without GPI anchored signal at C-terminus, signal sequence from MV Fusion protein at N-terminus, chemically synthesized; Genscript, USA) were inserted in ATU2, to produce respectively pTM2-MVSchw-CSPb and pTM2-MVSchw-CSPf plasmids. The sequences, which were codon optimized for expression in mammalian cells, respected the “rule of six”, which stipulates that the number of nucleotides in the MV genome must be a multiple of 6, and contain BsiWI/BssHII restriction sites at both ends. Rescue of both recombinant viruses (rMV-CSPb and rMV-CSPf) was performed as previously described (Combredet, et al. J. Virol. 2003, 77(21):11546-54) using the helper-cell-based rescue method described by Radecke et al. (Radecke, et al. EMBO J. 1995, 14(23):5773-84; Parks, et al., J. Virol. 1999, 73(5):3560-6) and modified by Parks et al. (Parks, et al., J. Virol. 1999, 73(5):3560-6). rMV-CSPb and rMV-CSPf were grown on Vero cells.

### Virus Growth Curves

Monolayers of Vero cells grown in 24-mm-diameter dishes (6-well plates) were infected with rMV-CSPb and rMV-CSPf at an MOI of 1. At various times post-infection, cells were scraped into culture medium. After freeze thawing of cells and medium, and clarification of cell debris, virus titers were determined by endpoint dilution assay. For this purpose, Vero cells were seeded into 96-well plates (7,500 cells/well) and infected with serial 1:10 dilutions of virus sample in DMEM-5% FCS. After incubation for 7 days, cells were stained with crystal violet, and the TCID50 values were calculated by use of the Spaerman-Kärber method (Spaerman Br. J. Psychol. 1908(2):227-42).

### Antigens Expression

Expression of CSPf and CSPb was assessed in Vero cells infected with rMV-CSPb and rMV-CSPf by IFA and Western blotting. IFA was performed on Vero cells at 36 hours post-infection with rMV-CSPb and rMV-CSPf at an MOI of 0.1. Cells were probed with 3D11 mouse anti-CSPb monoclonal antibody (1/1,000 dilution) (#MR4-100 hybridoma) or 2A10 mouse anti-CSPf monoclonal antibody (1/1,000 dilution) (#MR4-183 hybridoma). Cy3-conjugated goat anti-mouse IgG (Jackson immunoresearch laboratories) was used as secondary antibody (1/1,000 dilution). Western blotting was performed on infected Vero cell lysates fractionated by SDS-PAGE and transferred to cellulose membranes. 3D11 mouse anti-CSPb monoclonal antibody and 2A10 mouse anti-CSPf monoclonal antibody were used to detect CS proteins. A goat anti-mouse IgG-horseradish peroxidase (HRP) conjugate (#P0447, Dako) was used as secondary antibody.

### Mice Immunizations and Challenge

Mice deficient for type-I IFN receptor (IFNAR) and expressing human CD46 (hCD46) (Combredet, et al. J. Virol. 2003, 77(21):11546-54) were housed under pathogen-free conditions at the Institut Pasteur animal facility. Group of 6 six-week-old hCD46IFNAR mice were inoculated with different doses of MV-Schwarz, rMV-CSPb and rMV-CSPf, via the intraperitoneal route (i.p.). To study cellular response, only one immunization was administered and spleens were extracted eight days later. For humoral response and infectious challenge, two immunizations were administered within a 4 weeks interval. Sera were collected before the first immunization (day 0, negative control) and 3 weeks after each immunization, and 4 months after the second immunization to study long-term memory responses. Immunized mice were challenged with P. berghei ANKA sporozoites expressing the green fluorescent protein (GFP) under the control of the hsp70 promoter (Ishino, et al. Mol. Microbiol; 2006, 59(4):1175-84). Alternatively, mice immunized with rMV-CSPf were challenged with P. berghei NK65 sporozoites expressing the GFP under the control of the hsp70 promoter (Demarta-Gatsi, et al., J. Exp. Med. 2016, 213(8): 1419-28) and the CSPb harboring the central repetitive region of CSPf (rGFP-Pb-CSPf repeat) (Persson, et al. J. Immunol. 2002, 169(12): 6681-5). rGFP-Pb-CSPf repeat parasites were generated by a genetic cross as described by Ishino et al. (Ishino, et al. Mol. Microbiol. 2006, 59(4):1175-84). Sporozoites were freshly collected from the salivary gland of infected Anopheles stephensi in D-PBS and filtered using a 35pm nylon mesh cell strainer snap cap (Corning Falcon). Infectious challenges were executed 4 weeks after the second immunization (early response), or 4 months after the second immunization (long-term memory response) by the microinjection of 5,000 sporozoites in one microliter of D-PBS in the posterior footpad using a 35G microsyringe (World Precision Instruments). After challenge, parasitemia was monitored from day 3 to day 10. Blood samples (2 μl) were diluted in 500 μl of PBS and analyzed by flow cytometry (MacsQuant, Miltenyi Biotec). Doublets and clusters of red blood cells (RBCs) were excluded from counts. Single GFP+RBCs (infected RBC, iRBCs) among total RBCs were estimated and data analyzed by the MACSQuantify™ Software. As no protection against blood stage parasites was expected, mice were sacrificed at day 10 post-challenge in the presence of iRBCs in order to avoid unnecessary suffering, or before in the presence of severe symptoms that were ethical endpoints (signs of cerebral malaria: motor troubles, ruffled fur and sometimes convulsions). Non-parasitemic mice at day 10 were considered sterile protected. Experiments were conducted following the guidelines of the Office of Laboratory Animal Care at Institut Pasteur.

### ELISA

Measles virus antigen, Edmonston strain (#PR-BA102-S-L, Jena Bioscience) antigen at 1 μg/ml in PBS, and CSPb or CSPf recombinant proteins (produced at the Recombinant Protein and Antibodies Production Core Facility of the Institut Pasteur by J. Bellalou and V. Bondet, using the BioPod F800 microfermentor battery) at 1 pg/ml in carbonate buffer were coated overnight at 4° C. onto 96-well plates (#439454, Thermo Scientific) and then blocked for 1 h at 37° C. with a saturation buffer (PBS, 0.05% Tween, 3% BSA). Sera samples from immunized mice were serially diluted (PBS, 0.05% Tween, 1% BSA) and incubated on plates for 1 h at 37° C. After washing steps (0.05% Tween in PBS), a secondary horseradish peroxidase conjugated goat anti-mouse Ig antibody (#115-035-146, Jackson ImmunoResearch) was added at a dilution of 1/1,000 for 1h at 37° C. Antibody binding was revealed by addition of the TMB substrate (#5120-0047, Eurobio) and the reaction was stopped by addition of H₂SO₄ 1M. The optical densities (O.D.) were recorded at 450 nm. The endpoint titers for each individual serum were calculated as the reciprocal of the last dilution giving twice the absorbance of negative control sera.

### ELISPOT Assay

Freshly extracted splenocytes from immunized mice were tested for their capacity to secrete IFN-γ upon specific stimulation. Multiscreen-HA 96-well plates (#MSIP4510, Millipore) were coated overnight at 4° C. with 5 μg/ml of anti-mouse IFN-γ (#551216, BD Biosciences Pharmingen) in PBS and, after washing, were blocked for 2 h at 37° C. with complete MEM (MEM—10% FCS supplemented with non-essential amino-acids 1%, sodium pyruvate 1% and β-mercapto-ethanol). The medium was then replaced with 100 μl of cell suspension containing 2×10⁵ splenocytes in each well (triplicate) and 100 μl of stimulating agent in complete MEM. Plates were incubated for 40h at 37° C. Cells were stimulated with Concanavalin A (#C-5275, Sigma) as positive control, complete MEM as negative control, live attenuated MV-Schwarz virus at an MOI of 1, and CSPf recombinant protein at 50 μg/ml. After incubation and washing, biotinylated anti-mouse IFN-γ antibody (#554410, BD Biosciences Pharmingen) was added and plates were incubated for 90 minutes at room temperature. After extensive washing, streptavidin-alkaline phosphatase conjugate (#7100-05, Clinisciences) was added and plates were incubated 1 h at room temperature. Spots were developed with BCIP/NBT (#61911, Sigma) and counted in an ELISPOT reader (CTL ImmunoSpot®).

### Intracellular Cytokine Staining

Freshly extracted splenocytes from immunized mice were analyzed by flow cytometry for their capacity to secrete IFN-γ and TNF-α upon specific stimulation. Spleen cells were cultured for 16 hours in U-bottom 96-well plates (1.0×10⁶ cells/well) in a volume of 0.2 ml complete medium (MEM—10% FCS supplemented with non-essential amino-acids 1%, sodium pyruvate 1% and β-mercapto-ethanol). Cells were stimulated with PMA/ionomycin (#00-4970, ebioscience) as positive control, complete MEM as negative control, live attenuated MV-Schwarz virus at an MOI of 1, and CSPf LPS-free recombinant protein at 50 μg/ml. Brefeldin A (#66542, Sigma) was then added at 10 μg/ml for 6 more hours of incubation. Stimulated cells were harvested, washed in phosphate-buffered saline containing 1% bovine serum albumin and 0.1% w/w sodium azide (FACS buffer), incubated 10 minutes with Fc blocking Ab (CD16/32 clone 2.4G2, PharMingen) and surface stained in FACS buffer with Live/Dead fixable dead cell violet kit (#L34955, invitrogen), anti-mouse CD4-PECy7 mAb (#552775, BD Biosciences) and anti-mouse CD8-APCH7 mAb (#560182, BD Biosciences) for 30 minutes at 4° C. in the dark. After washing, cells were fixed and permeabilised for intracellular cytokine staining using the Cytofix/Cytoperm kit (#554922, BD Bioscience). Cells were then incubated in a mix of anti-mouse IFNγ-APC mAb (#554413, BD Biosciences) and anti-mouse TNF-α-FITC mAb (#554418, BD Biosciences) diluted in permwash buffer (#557885, BD Bioscience) for 30 minutes in the dark. After washing with permwash buffer and FACS buffer, cells were fixed with 1% formaldehyde in PBS. Data were acquired using a MacsQuant® Analyzer (Miltenyi Biotec), and analysed using Flow Jo™ 9.3.2 software and are presented as % of CD4+ or CD8+ cells expressing TNF-α or IFNγ among total CD4 or CD8 populations.

### Statistical Analysis

Parasitemia was Log transformed for normalization. Statistical analyses were done using the t-test. Differences were considered statistically significant when p<0.05.

### EXAMPLE 1

### Production of rMVs Expressing CSPb and CSPf Proteins

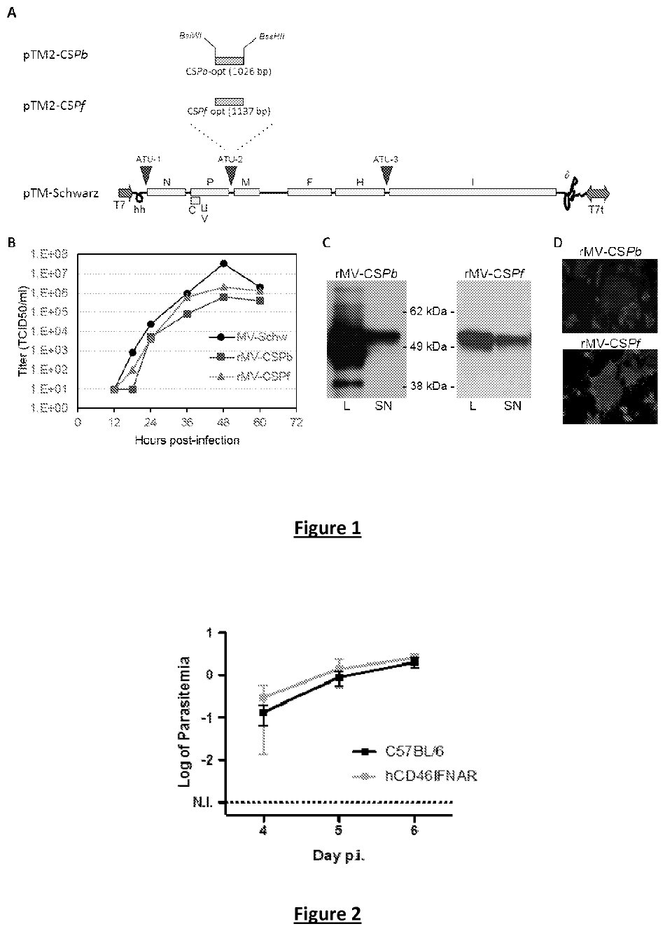

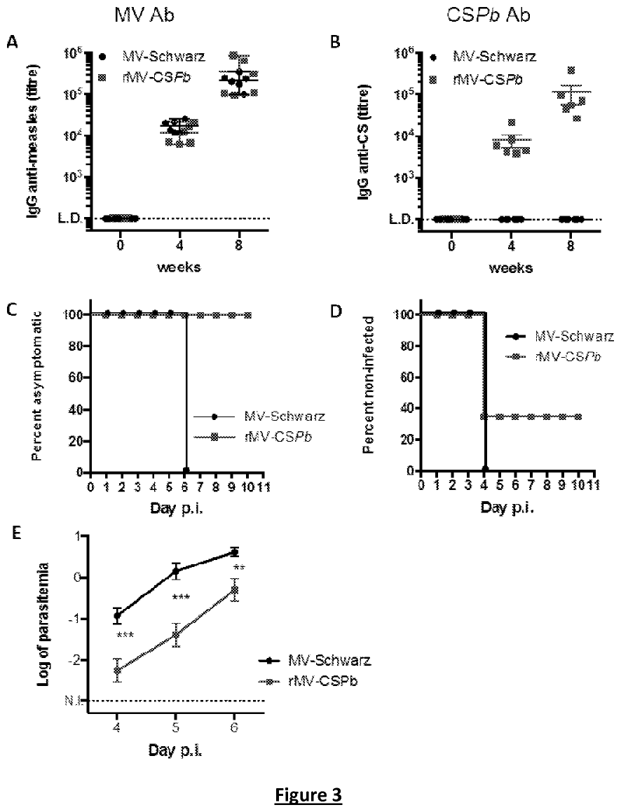

The inventors constructed an rMV expressing CSPb protein (rMV-CSPb) and an rMV expressing CSPf protein (rMV-CSPf) by inserting mammalian codon-optimized sequences of both proteins in additional transcription unit 2 (ATU2) of pTM-MVSchw plasmid, which encodes the antigenome of the Schwarz MV vaccine strain (Combredet, et al. J. Virol. 2003, 77(21):11546-54) (FIG. 1A). The ATU2 allowed high-level expression of the protein, as there was a decreasing gradient of gene expression generated by MV replication (from high nucleoprotein “N” expression to low polymerase “L” expression). Both plasmids were transfected into HEK293T-helper cells for rescue and co-cultured with Vero cells for virus spread. The rescued rMV-CSPb and rMV-CSPf had slightly delayed growth curves, as compared to empty MV (FIG. 1B), but still reached high titers on Vero cells. Viral stocks were made from unique syncytia after rescue and were therefore considered as clonal. The expression of CS was assessed by Western blot, and found in the lysate and in the supernatant of infected Vero cells (FIG. 1C). The CS expression in infected cells forming syncytia was also demonstrated by immunofluorescence (FIG. 1D). For rMV-CSPf, the stability of transgene expression was demonstrated after 10 passages of the recombinant virus on Vero cells by immunofluorescence, Western blot and sequencing (data not shown). The stability of rMV-CSPb was not tested as the mouse model was only used for proof of concept.

### EXAMPLE 2

### Susceptibility of hCD46IFNAR Mice to Plasmodium berghei ANKA Challenge

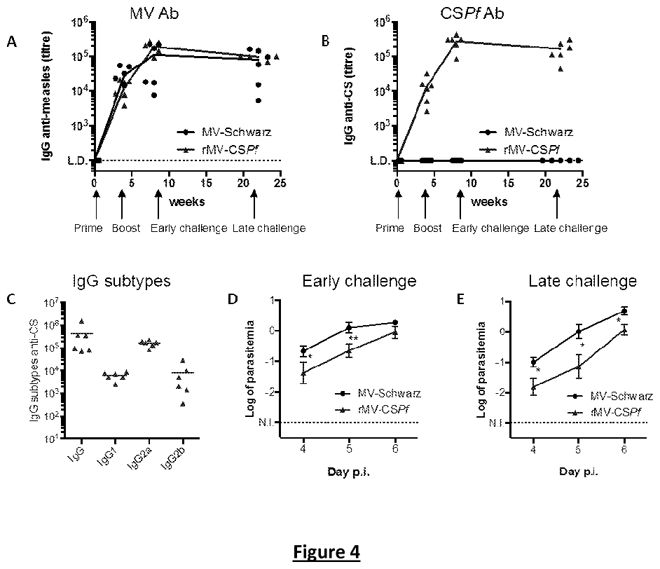

Mice are naturally resistant to MV, which is restricted to human and non-human primates. The usual mouse model to test rMV vaccine candidates is deficient for type-I IFN receptor (IFNAR) and expresses human receptor CD46 (hCD46) (Combredet, et al. J. Virol. 2003, 77(21):11546-54). The genetic background of hCD46IFNAR mouse used in the present invention was Sv129, which had the same major histocompatibility complex haplotype as C57BL/6 mouse (H-2Db, H-2Kb, I-Ab). C57BL/6 mice infected with P. berghei ANKA (PbA) was a model for cerebral malaria, which lead to death. C57BL/6 mice were easily infected and highly susceptible, as compared to Balb/c mice (Jaffe, et al. Am. J. Trop. Med. Hyg. 1990, 42(4):309-13; Hafalla, et al. PLoS Pathog. 2013, 9(5):e1003303). In order to validate the model of infection in hCD46IFNAR mice, the inventors inoculated 5,000 GFP-expressing PbA (GFP PbA) sporozoites in the footpad of six C57BL/6 and six hCD46IFNAR mice. The inventors monitored the parasitemia from day 4 to day 6 post-inoculation. Mice were sacrificed at day 6 post-challenge in the presence of iRBCs in order to avoid unnecessary suffering (ethical endpoints). Although parasitemia was slightly higher in hCD46IFNAR group, there was no statistically significant difference between both groups of mice (FIG. 2). So, the inventors concluded that both mouse models were comparable for sporozoite challenge. These observations validated the use of hCD46IFNAR mouse for the rest of the study.

### EXAMPLE 3

### Immunogenicity and Protective Efficacy of rMV-CSPb as a Proof of Concept

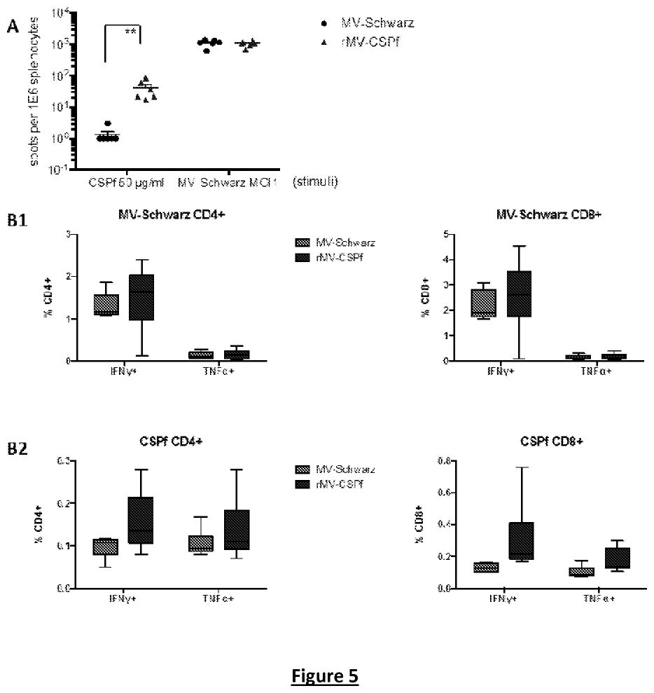

Six-week-old hCD46IFNAR mice (6 mice per group) received 10⁵ TCID50 of rMV-CSPb, or MVSchw as negative control, by intraperitoneal (i.p.) route at day 0 and at day 28. Sera were collected before the first immunization (control) and 3 weeks after each immunization. Antibodies to MV were induced at similar levels in all immunized mice (FIG. 3A). Antibodies to CSPb were efficiently induced from the first immunization with limiting dilution titers of about 10⁴, then boosted after the second immunization to reach 10⁵ (FIG. 3B). Mice were challenged 3 weeks after the second immunization with 5,000 sporozoïtes of GFP-PbA injected in the footpad. In MVSchw immunized group (control), the inventors sacrificed mice at day 6 post-challenge (FIG. 3C), due to start of cerebral symptoms, which were ethical endpoints of the study. In rMV-CSPb immunized group, two mice (33%) achieved sterile protection (no detectable iRBC at day 10 post-challenge) (FIG. 3D). The other mice showed a significant delayed and decreased parasitemia (FIG. 3E), with no observed severe symptoms. Moreover, at day 10 post-challenge, the parasitemia in rMV-CSPb immunized mice was still less than 1%. So, immunization with rMV-CSPb achieved sterile protection in 33% of hCD46IFNAR mice and completely protected mice from severe and lethal PbA-induced cerebral malaria.

### EXAMPLE 4

### Immunogenicity of rMV-CSPf: Thi IgG Subtype Profile and Long-Term Memory

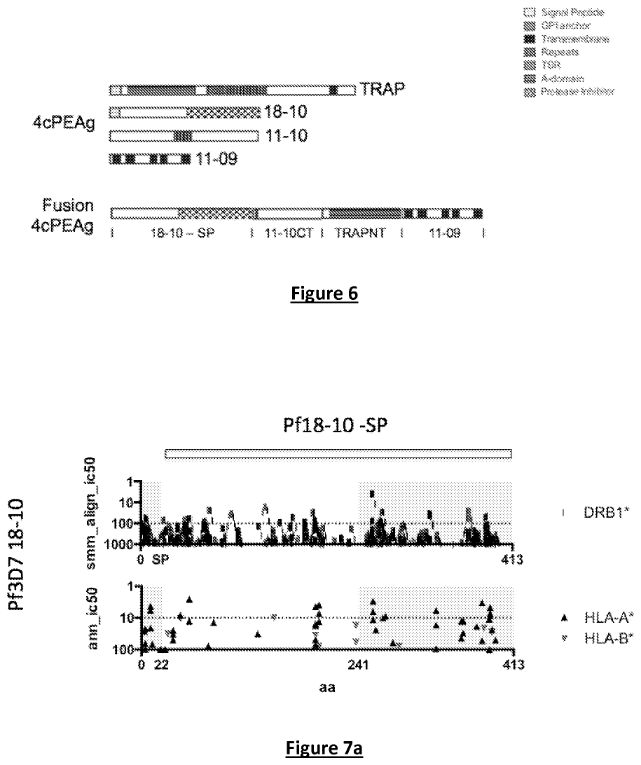

Six-week-old hCD46IFNAR mice (6 mice per group) received 10⁵ TCID50 of rMV-CSPf, or MVSchw as negative control, by intraperitoneal (i.p.) route at day 0 and at day 28. Sera were collected before the first immunization (control), 3 weeks after each immunization, and 22 weeks after the first immunization for a group of 6 mice dedicated to long-term memory study. As for rMV-CSPb, antibodies to MV were induced at similar levels in all immunized mice (FIG. 4A) and antibodies to CSPf were efficiently induced from the first immunization with limiting dilution titers of about 10⁴, then boosted after the second immunization to reach 10⁵ (FIG. 4B). Interestingly, this high antibody titer was maintained 22 weeks post-prime. The humoral response profile corresponded to Th1 polarization with high titers of IgG2a antibodies (FIG. 4C), as expected for a replicative viral vector. Mice were challenged 3 weeks after the second immunization (early challenge) or 22 weeks post-prime (late challenge) with 5,000 sporozoites of recombinant GFP-Pb expressing CSPb with CSPf repeat sequence (rGFP-Pb-CSPf repeat), microinjected in the mouse footpad. In MVSchw immunized group (control), all mice were sacrificed at day 6 post-challenge, due to start of cerebral symptoms, which were ethical endpoints of the study. In rMV-CSPf immunized group, there was no induction of sterile protection, but a decreased and delayed parasitemia, whether for early (FIG. 4D) or late challenge (FIG. 4E). Mice started to present symptoms of cerebral malaria at day 7 and were sacrificed to avoid unnecessary suffering. This decreased parasitemia was therefore less important than the one observed for rMV-CSPb. The inventors hypothesized that the observed difference was due to the challenge model with rGFP-PbA-CSPf repeat that allow only to study protection relying on neutralizing antibodies directed against the repeat sequence. The inventors therefore evaluated the cellular response in the Pf model.

### EXAMPLE 5

### Induction of Specific Cellular Immune Response

Cell-mediating immune response (CMI) elicited by immunization with rMV-CSPf was assessed using IFNy Elispot assay and intracellular cytokine staining (IFNγ and TNFα) on freshly extracted splenocytes collected 7 days after a single immunization with 1.10⁵ TCID50 in 100 μl i.p. (FIG. 5). Splenocytes were stimulated ex vivo with inactivated MV-Schwarz at an MOI of 1 or CSPf recombinant LPS-free protein at 50 μg/ml. A moderate but significant (p<0.01) number of CSPf-specific cells (up to 100/10⁶ splenocytes) were detected by the ELISPOT assay (FIG. 5A), which corresponds to 5-10% of the number of MV-specific spots. The phenotype of MV- and CSPf-specific cells induced by rMV-CSPf was analyzed by flow cytometry (FIG. 5B). The mean frequency of MV-specific T cells secreting IFNγ and TNFα in CD4+ cells (B1 left panel) was respectively 1.5% and 0.2%. The mean frequency of MV-specific T cells secreting IFNγ and TNFα in CD8+ cells (B1 right panel) was respectively 2.6% and 0.2%. The mean frequency of CSPf-specific T cells secreting IFNγ and TNFα in CD4+ cells (B2 left panel) was respectively 0.16% and 0.14%. The mean frequency of MV-specific T cells secreting IFNγ and TNFα in CD8+ cells (B2 right panel) was respectively 0.3% and 0.18%. An induction of CD4+ cells secreting IFNγ and CD8+ cells secreting IFNγ or TNFα was observed, as compared to control group but statistically not significant (p=0.083, p=0.088 and p=0.057 respectively). Even if no CD8+ epitopes of CSPf were described in C57BL/6 mouse, the inventors showed the induction of a moderate but significant CMI as early as 7 days after a single immunization with rMV-CSPf, with CD4+ and CD8+ activated phenotype.

### EXAMPLE 6

### Construction of a Chimeric Antigen of a Plasmodium Parasite

The identification of the protective domains (PD) of four pre-erythrocytic conserved protective antigens allowed the construction of a chimeric antigen formed by the fusion in this order of a fragment of the ICP (18-10) of a Plasmodium parasite, a fragment of the protein Ag45 (11-10) of a Plasmodium parasite, a fragment of the TRAP of a Plasmodium parasite, and the protein Ag40 (11-09) of a Plasmodium parasite or a fragment thereof.

For example, a chimeric antigen of Plasmodium berghei ANKA have been constructed by the fusion in this order of the PD Pb18-10NT of SEQ ID NO: 15, the PD Pb18-10CT of SEQ ID NO: 17, the PD Pb11-10CT of SEQ ID NO: 21, the PD PbTRAPNT of SEQ ID NO: 25 and the antigen Pb11-09 of SEQ ID NO: 29. This chimeric antigen was called P. berghei Fusion 4cPEAg (SEQ ID NO: 41 or 43) and its structure is shown in FIG. 6.

As another example, a chimeric antigen of Plasmodium berghei ANKA has been constructed by the fusion in this order of the PD Pb18-10NT of SEQ ID NO: 15 and the PD Pb18-10CT if SEQ ID NO: 17.

As another example, a chimeric antigen of Plasmodium falciparum 3D7 have been constructed by the fusion in this order of the PD Pf18-10 of SEQ ID NO: 19, the PD Pf11-10CT of SEQ ID NO: 23, the PD PfTRAPNT of SEQ ID NO: 27 and the antigen Pf11-09 of SEQ ID NO: 31. This chimeric antigen was called P. falciparum Fusion 4cPEAg (SEQ ID NO: 45 or 47).

As another example, a chimeric antigen of Plasmodium falciparum 3D7 have been constructed by the insertion of the full antigen ICP 18-10 devoid of its signal peptide, in particular ICP 18-10 of SEQ ID NO: 19.

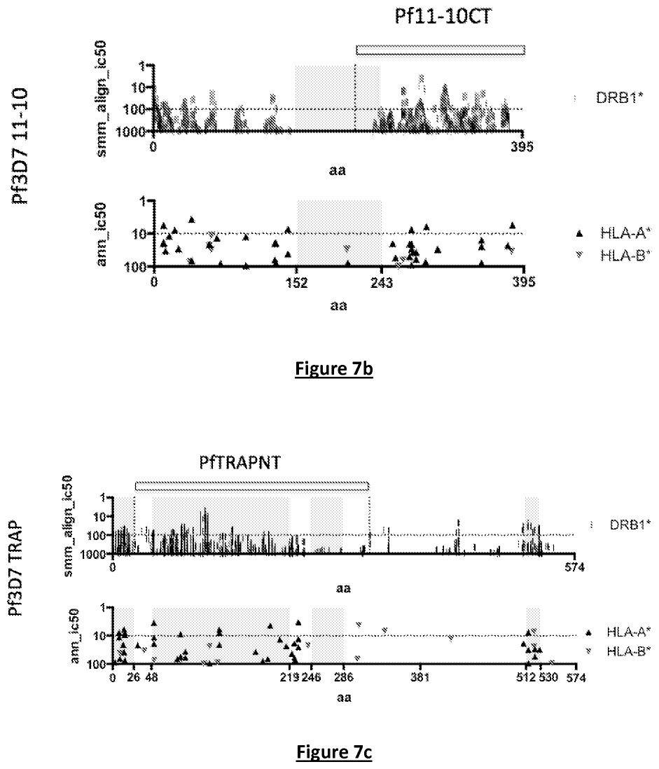

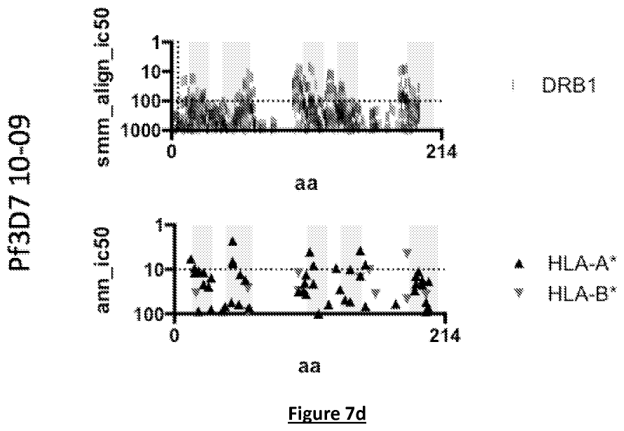

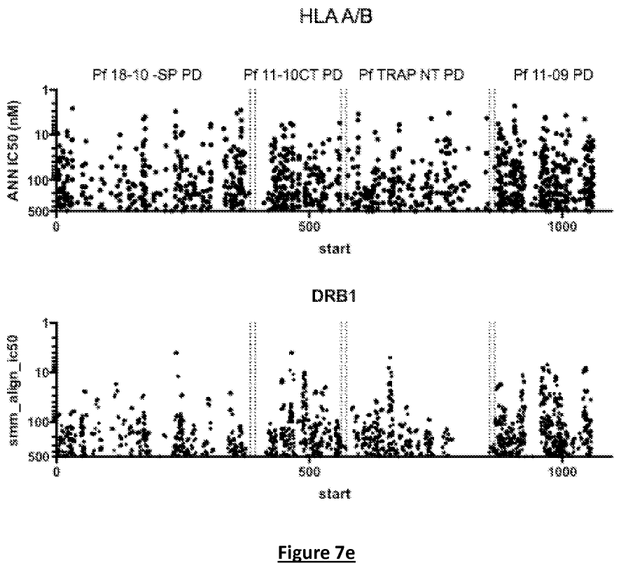

Since predicted CD8+T cell epitopes clustered in conserved regions of the antigens, independently of the plasmodial species and MHC class I restriction, this particularity was used to select the regions of P. falciparum 4cPEAg, corresponding to the protective domains of P. berghei 4cPEAg. More HLA class I and II allelles were analyzed, including the mapping of 9-mers peptides predicted to bind to HLA-DRB1*01:01, *03:01, *04:01, *04:05, *07:01, *08:02, *09:01, *11:01, *12:01, *13:02 and *15:01, to the HLA A*01:01, *02:01, *02:03, *02:06, *03:01, *11:01, *23:01,*24:02, *26:01, *30:01, *30:02, *31:01, *32:01, *33:01, *68:01 and *68:02., and to the HLA-B*07:02, *08:01, *15:01, *35:01, *40:01, *44:02, *44:03, *51:01, *53:01, *57:01 and *58:01 (FIG. 7). This extended analysis corroborated the initial observation, using the HLA A*02:01 and the H2Db/Kb, that good binders tend to cluster in regions associated with structural/functional conserved domains, transmembrane domains, as well as in signal peptide and GPI-anchoring sequences. Based on this clustering of epitopes, the sequences/structures of P. berghei antigens were used to retrieve the cognate regions in P. falciparum antigens as shown in FIG. 7. These putative protective domains were fused as in P. berghei avoiding the creation of neo-epitopes in the junction of antigens/protective domains as shown in FIG. 7e. When inevitable, an amino acid residue was introduced in the fusion sequence to avoid the creation of neo-epitopes with high binding affinity to HLA. The only amino acid added in the Pf fusion 4cPE Ag was a glutamic acid (E) at the end of the Pf11-10CT.

### EXAMPLE 7

### Plasmodium Berghei Infectious Challenge in hCD46IFNAR Mice

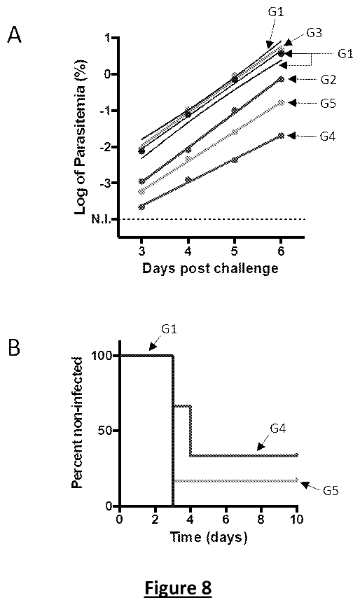

As shown in FIG. 8(A), the expression of CSPb alone (G2) reduced the parasitemia, as compared to the control group (G1). The expression of Pb Fusion alone (G3) had no effect on parasitemia. However, when Pb Fusion was expressed simultaneously to CSPb (G4 and G5), the parasitemia was highly decreased. The effect was higher when both antigens were expressed in the same virus (G4) than in two viruses injected separately (G5) where a competitive effect could not be excluded.

As shown in FIG. 8(B), mice from G4 (33%) and G5 (17%) showed sterile protection, i.e. no blood infection at day 10 post-challenge. The expression of a single malaria antigen, either CSPb (G2) or Pb Fusion (G3) was not able to achieve sterile protection in this experiment.

This experiment clearly showed the synergistic effect of both antigens to achieve protection.

### Discussion

Following the moderate protection and short memory response induced by RTS,S vaccine candidate in phase III clinical trial (Aaby, et al. Lancet 2015, 386(10005):1735-6), there is strong support for developing a second-generation malaria vaccine with higher efficacy and longer duration of protection. Because of its central place in infant vaccine schedules all over the world, measles provides a promising viral vector to deliver malaria antigens, either as a single delivery platform or in a prime boost strategy. The inventors have reported the use of measles-based vaccine platform to deliver CS malaria antigen as a proof of concept of the feasibility and advantages of this vector, in a murine model. Importantly, the inventors showed induction of cellular response and long-term memory with high antibody titers. These are the two main characteristics required for second-generation malaria vaccine candidates.

The inventors first showed the possibility of stably expressing a malaria antigen using the measles virus as a delivery vector. CSPb and CSPf sequences were successfully inserted in MV-Schwarz genome and stably maintained after 10 passages in Vero cell culture, without any mutation. Nevertheless, the inventors were unable to rescue a virus with CS native sequence (data not shown) and therefore mammalian codon-optimized sequence is required. The P. falciparum genome is AT rich (Gardner, et al. Nature 2002, 419(6906):498-511) and polyA/polyU probably disturbed measles polymerase, either for replication or transcription. As MV-Schwarz vector is able to insert 6 kb in its additional transcription units, other antigens could be easily added to CS to improve vaccine efficacy.

Then the inventors showed in the hCD46IFNAR mouse model the induction of high antibody titers that were maintained at least until 22 weeks post-prime in a two-immunization schedule with one-month interval. This maintenance of high antibody level was longer than the one observed with CS administered in a three doses regimen at 50 μg with complete Freund's adjuvant in C57BL/6 mice (Wirtz, et al. Exp. Parasitol. 1987, 63(2):166-72), whereas rMV delivered only ng of heterologous antigens (Brandler, et al. PLoS Negl. Trop. Dis. 2007, 1(3):e96). R16HBsAg, a precursor of RTS,S, induced high level of antibodies in mice when administered with alum in a three dose regimen, but was not assessed more than 5 weeks after the last immunization (Rutgers, et al., Nat. Biotechnol. 1988, 6:1065-70). In monkeys, RTS,S/AS01B formulation has shown a rapid decrease of CS antibodies 8 weeks after each boost (Mettens, et al. Vaccine 2008, 26(8):1072-82). The long-term persistence of neutralizing antibodies against heterologous antigens vectored by rMV has already been described for an rMV expressing HIV antigens in both mouse (Guerbois, et al. Virol. 2009, 388(1):191-203) and non-human primate (NHP) models (Stebbings, et al. PLoS One, 2012, 7(11):e50397). Thus, the observed maintenance of high anti-CS antibody level is promising regarding MV efficiency to induce life-long memory. IgG sub-types were predominantly IgG2a, which was expected for a replicating viral vector. This subclass is cytophilic in mice (Waldmann, et al. Annu. Rev. Immunol. 1989, 7:407-44), with complement fixation and pathogen opsonization. Moreover, induction of cytophilic CS antibodies has been associated with protection from re-infection in the field (John, et al. Am. J. Trop. Med. Hyg. 2005, 73(1):222-8). Nevertheless, it is important to remember that the parasite itself escapes immunity by modulating immune responses (Wykes, et al. EMBO Rep. 2013, 14(8):661). Thus, further investigations of memory B cells' survival (Liu, et al. Eur. J. Immunol. 2012, 42(12):3291-301) and dendritic cells' functionality (Wykes, et al. Nat. Rev. Microbiol. 2008, 6(11):864-70) after infectious challenge would help identify predictive factors of long-term efficacy in human.

To evaluate protection, the inventors used C57BL/6 mice and PbA model, which was a relevant model of liver stage immunity that closely resembles the situation in humans. In this model, sterile protection was not as easy as for Balb/c mice, where CSPb was target of immuno-dominant and protective CD8+ T cell response (Romero, et al. Nature 1989, 341(6240):323-6). Indeed, CS seemed to contain no naturally processed and presented H-2b restricted epitopes (Hafalla, et al. PLoS Pathog. 2013, 9(5):e1003303). Sv129 hCD46IFNAR mice and C57BL/6 mice both expressed H-2b major histocompatibility complex. The inventors showed that they were similarly sensitive to PbA challenge, with similar clinical features and no statistical difference in parasitemia on days 3, 4, 5 and 6 post-infection. Palomo et al. showed a slightly delayed experimental cerebral malaria development and prolonged survival of C57BL/6 IFNAR mice, as compared to wild-type mice (Palomo, et al. Eur. J. Immunol. 2013, 43(10):2683-95). Nevertheless, the inventors defined ethical endpoints at the beginning of the study that had imposed an early sacrifice at day 6 or 7 post-infection, and the inventors did not wait for natural death to avoid unnecessary suffering. rMV-CSPb was able to elicit sterile protection in 33% of mice and to protect all of them from severe disease, with a reduced and delayed parasitemia, and no severe clinical symptoms. In the rGFP-PbA-CSPf repeat challenge model, there was no sterile protection and reduction in parasitemia was less compared to the PbA model. This suggested that sterile protection was not induced by neutralizing antibodies directed against the repeat sequence of CSPf, but might involve antibodies against C and N-terminal domains of CS and cell-mediated immune responses. In fact, phagocytic activity of antibodies induced by RTS,S/AS01 malaria vaccine had been correlated with full-length CS and C-terminal specific antibody titer, but not to repeat region antibody titer (Chaudhury, et al. Malar. J. 2016, 15:301). Accordingly, the inventors showed a moderate but significant induction of cell-mediated immune response that appeared as early as 7 days after a single immunization, with an increase in CD4+ and CD8+ specific T cells secreting IFNγ or TNFα. As there was no described CD8+ epitope for CSPf in H-2b mice, the increase observed, even if moderate, was of great interest. Indeed, protection against malaria had been correlated to CSPf CD8+ T cell response in human immune system (HIS) mice harboring functional human CD8+ T cells (Li, et al. Vaccine, 2016, 34(38):4501-6). This major role for CD8+ T cells to induce protection was already shown by in vivo depletion of CD8+ T cells that abrogated sporozoite-induced protective immunity in mice (Weiss, et al. PNAS 1988, 85(2):573-6). Thus, even if the protection resulting from rGFP-PbA-CSPf repeated challenge model was not indicative of real protection, it brought indications of efficient immune mechanisms involved in protection.

To conclude, the inventors demonstrated the promising potential of using measles vector to deliver malaria antigens by showing induction of cellular immune responses and long-term memory with high antibody titers in mice. These are two critical desired characteristics for second-generation malaria vaccines. As expected, expression of CS alone was not able to induce sterile protection in this mouse model and the inventors had used it only as a ‘gold standard’ to validate their viral vector. Further recombinant measles-vectored malaria vaccine candidates expressing additional pre-erythrocytic and/or blood-stage antigens in combination with CS is under evaluation. It remains to be seen if such combinations yield synergistic effects to provide protection with higher efficacy and for longer duration. rMV-vectored malaria vaccine candidates expressing additional pre-erythrocytic and/or blood-stage antigens in combination with rMV expressing PfCS may provide a path to development of next generation malaria vaccines with higher efficacy.

1. A chimeric measles virus (MV)-based nucleic acid construct suitable for the expression of heterologous polypeptides, which comprises:
a cDNA molecule encoding a full-length, infectious antigenomic (+) RNA strand of a MV; and
(1) a first heterologous polynucleotide encoding at least the circumsporozoite (CS) protein of a Plasmodium parasite or an antigenic fragment thereof; and
(2) a second heterologous polynucleotide encoding at least a chimeric antigen of a Plasmodium parasite; and
wherein said chimeric antigen as defined in (2) comprises or consists of the following fragments of (a), (b), (c) and (d) assembled in a fusion polypeptide, wherein the fragments of (a), (b), (c) and (d) elicit a human leukocyte antigen (HLA)-restricted CD8⁺ and/or CD4⁺ T cell response against a Plasmodium parasite, and are directly or indirectly fused in this order:
(a) a fragment of the inhibitor of cysteine protease (ICP) (18-10) of a Plasmodium parasite,
(b) a fragment of the protein Ag45 (11-10) of a Plasmodium parasite,
(c) a fragment of the thrombospondin related anonymous protein (TRAP) of a Plasmodium parasite, and
(d) the protein Ag40 (11-09) of a Plasmodium parasite or a fragment thereof,
or a chimeric antigen variant thereof, which consists of a chimeric antigen having an amino acid sequence which has at least 90% sequence identity or more than 95% sequence identity or 99% sequence identity with the sequence of the fusion polypeptide consisting of fused fragments of (a), (b), (c) and (d), from which it derives by point mutation of one or more amino acid residues, over its whole length;
wherein the first heterologous polynucleotide is operatively linked, in particular cloned within an additional transcription unit (ATU) inserted within the cDNA molecule; and
wherein the second heterologous polynucleotide is operatively linked, in particular cloned within an ATU inserted within the cDNA molecule at a location distinct from the location of the first linked, in particular cloned heterologous polynucleotide.

2. The nucleic acid construct according to claim 1, further comprising a third heterologous polynucleotide encoding at least the reticulocyte-binding protein homologue 5 (RH5) of a Plasmodium parasite or an antigenic fragment thereof, wherein said third heterologous polynucleotide is directly fused or indirectly fused to the first heterologous polynucleotide.

3. The nucleic acid construct according to claim 1, wherein said nucleic acid construct complies with the rule of six of the MV genome.

4. The nucleic acid construct according to claim 1, comprising the following polynucleotides encoding polypeptides from 5′ to 3′:
(a) a polynucleotide encoding the N protein of the MV;
(b) a polynucleotide encoding the P protein of the MV;
(c) the first heterologous polynucleotide encoding at least the CS protein of the Plasmodium parasite or the antigenic fragment thereof;
(d) a polynucleotide encoding the M protein of the MV;
(e) a polynucleotide encoding the F protein of the MV;
(f) a polynucleotide encoding the H protein of the MV;
(g) the second heterologous polynucleotide encoding the at least a chimeric antigen of the Plasmodium parasite; and
(h) a polynucleotide encoding the L protein of the MV;
wherein said polynucleotides are operatively linked in the nucleic acid construct and under the control of viral replication and transcription regulatory sequences such as MV leader and trailer sequences.

5. The nucleic acid construct according to claim 2, comprising the following polynucleotides encoding polypeptides from 5′ to 3′:
(a) a polynucleotide encoding the N protein of the MV;
(b) a polynucleotide encoding the P protein of the MV;
(c) the first heterologous polynucleotide encoding at least the CS protein of the Plasmodium parasite or the antigenic fragment thereof;
(d) the third heterologous polynucleotide encoding at least the RH5 of the Plasmodium parasite or the antigenic fragment thereof, which is directly fused or indirectly fused to the first heterologous polynucleotide of (c);
(e) a polynucleotide encoding the M protein of the MV;
(f) a polynucleotide encoding the F protein of the MV;
(g) a polynucleotide encoding the H protein of the MV;
(h) the second heterologous polynucleotide encoding the at least a chimeric antigen of the Plasmodium parasite; and
(i) a polynucleotide encoding the L protein of the MV;
wherein said polynucleotides are operatively linked in the nucleic acid construct and under the control of viral replication and transcription regulatory sequences such as MV leader and trailer sequences.

6. The nucleic acid construct according to claim 1, wherein said measles virus is an attenuated virus strain selected from the group consisting of the Schwarz strain, the Zagreb strain, the AIK-C strain and the Moraten strain.

7. The nucleic acid construct according to claim 1, wherein the Plasmodium parasite is Plasmodium falciparum or Plasmodium berghei.

8. The nucleic acid construct according to claim 7, wherein the Plasmodium parasite is Plasmodium falciparum and wherein said first heterologous polynucleotide encoding at least the CS protein of Plasmodium falciparum or the antigenic fragment thereof further encodes (i) the signal peptide from the F protein of the MV or (ii) the signal peptide from the F protein of the MV and the intracytoplasmic and transmembrane domains of the F protein of the MV.

9. The nucleic acid construct according to claim 1, wherein the second heterologous polynucleotide encoding the at least a chimeric antigen of the Plasmodium parasite further encodes (i) the signal peptide from the F protein of the MV.

10. The nucleic acid construct according to claim 2, wherein the third heterologous polynucleotide encoding at least the RH5 of the Plasmodium parasite or the antigenic fragment thereof further encodes (i) the signal peptide from the F protein of the MV or (ii) the signal peptide from the F protein of the MV and the signal peptide from the F protein of the MV and the intracytoplasmic and transmembrane domains of the F protein of the MV.

11. The nucleic acid construct according to claim 1, wherein the fragment of the ICP (18-10) of the Plasmodium parasite of (a) has the amino acid sequence selected from the group consisting of SEQ ID NO: 14, SEQ ID NO: 16 and SEQ ID NO: 18, the fragment of the protein Ag45 (11-10) of the Plasmodium parasite of (b) has the amino acid sequence of SEQ ID NO: 20 or SEQ ID NO: 22, the fragment of the TRAP of the Plasmodium parasite of (c) has the amino acid sequence of SEQ ID NO: 24 or SEQ ID NO: 26, and the protein Ag40 (11-09) of the Plasmodium parasite or the fragment thereof of (d) has the amino acid sequence of SEQ ID NO: 28 or SEQ ID NO: 30.

12. The nucleic acid construct according to claim 1, wherein the first heterologous polynucleotide encoding at least the CS protein of the Plasmodium parasite or the antigenic fragment thereof has a sequence selected from the group consisting of SEQ ID NO: 8, SEQ ID NO: 10 and SEQ ID NO: 12, and wherein the second heterologous polynucleotide encoding the at least a chimeric antigen of the Plasmodium parasite has a sequence selected from the group consisting of SEQ ID NO: 40, SEQ ID NO: 42, SEQ ID NO: 44 and SEQ ID NO: 46.

13. The nucleic acid construct according to claim 2, wherein the third heterologous polynucleotide encoding at least the RH5 of the Plasmodium parasite or the antigenic fragment thereof has the sequence of SEQ ID NO: 32 or the sequence of SEQ ID NO: 34 or the sequence of SEQ ID NO: 56 or the sequence of SEQ ID NO: 58.

14. The nucleic acid construct according to claim 1, wherein the first heterologous polynucleotide encodes the CS protein of the Plasmodium parasite or the antigenic fragment thereof whose sequence is selected from the group consisting of SEQ ID NO: 9, SEQ ID NO: 11 and SEQ ID NO: 13, and the second heterologous polynucleotide encodes the chimeric antigen of the Plasmodium parasite whose sequence is selected from the group consisting of SEQ ID NO: 41, SEQ ID NO: 43, SEQ ID NO: 45 and SEQ ID NO: 47.

15. The nucleic acid construct according to claim 2, wherein the third heterologous polynucleotide encodes the RH5 of the Plasmodium parasite or the antigenic fragment thereof whose sequence is SEQ ID NO: 33 or SEQ ID NO: 35 or SEQ ID NO: 57 or SEQ ID NO: 59.

16. The nucleic acid construct according to claim 1, wherein said nucleic acid construct comprises a first polynucleotide whose sequence is selected from the group consisting of SEQ ID NO: 8, SEQ ID NO: 10 and SEQ ID NO: 12, and a second polynucleotide whose sequence is selected from the group consisting of SEQ ID NO: 40, SEQ ID NO: 42, SEQ ID NO: 44 and SEQ ID NO: 46.

17. The nucleic acid construct according to claim 2, wherein said nucleic acid construct comprises a first polynucleotide whose sequence is SEQ ID NO: 36 or SEQ ID NO: 38, and a second polynucleotide whose sequence is selected from the group consisting of SEQ ID NO: 40, SEQ ID NO: 42, SEQ ID NO: 44 and SEQ ID NO: 46.

18. A transfer vector plasmid, comprising the nucleic acid construct according to claim 1.

19. The transfer vector plasmid according to claim 18, whose sequence is SEQ ID NO: 54 or SEQ ID NO: 55.

20. Transformed cells comprising inserted in their genome the nucleic acid construct according to claim 1.

21. Recombinant infectious replicating measles virus (MV)-malaria virus particles, which comprise as their genome a nucleic acid construct according to claim 1.

22. Recombinant infectious replicating MV-malaria virus particles according to claim 21, which are rescued from a helper cell line expressing an RNA polymerase recognized by said cell line, for example a T7 RNA polymerase, a nucleoprotein (N) of a MV, a phosphoprotein (P) of a MV, and optionally an RNA polymerase large protein (L) of a MV.

23. The recombinant infectious replicating MV-malaria virus particles according to claim 21, wherein said virus particles comprise in their genome a polynucleotide sequence comprising (i) a first polynucleotide whose sequence is selected from the group consisting of SEQ ID NO: 8, SEQ ID NO: 10 and SEQ ID NO: 12, and a second polynucleotide whose sequence is selected from the group consisting of SEQ ID NO: 40, SEQ ID NO: 42, SEQ ID NO: 44 and SEQ ID NO: 46, or (ii) a first polynucleotide whose sequence is SEQ ID NO: 36 or SEQ ID NO: 38, and a second polynucleotide whose sequence is selected from the group consisting of SEQ ID NO: 40, SEQ ID NO: 42, SEQ ID NO: 44 and SEQ ID NO: 46.

24. A composition or an assembly of immunologically active ingredients comprising the recombinant infectious replicating MV-malaria virus particles according to claim 21 and a pharmaceutically acceptable vehicle.

25. A method for eliciting elicitation of antibodies directed against said proteins of a Plasmodium parasite, and/or of a cellular immune response, in a host, comprising administering the composition or the assembly of immunologically active ingredients according to claim 24 to the host.

26. A method for the prevention of a Plasmodium infection in a subject or in the prevention of clinical outcomes of infection by a Plasmodium parasite in a subject, in particular in a human comprising administering the recombinant infectious replicating MV-malaria virus particles according to claim 21 to the subject.

27. A process to rescue recombinant infectious replicating measles virus (MV)-malaria virus particles expressing (i) at least the circumsporozoite (CS) protein of a Plasmodium parasite or an antigenic fragment thereof, and at least a chimeric antigen of a Plasmodium parasite, or (ii) at least the CS protein of a Plasmodium parasite or an antigenic fragment thereof, at least a chimeric antigen of a Plasmodium parasite and at least the reticulocyte-binding protein homologue 5 (RH5) of a Plasmodium parasite or an antigenic fragment thereof,
wherein said chimeric antigen comprises or consists of the following fragments of (a), (b), (c) and (d) assembled in a fusion polypeptide, wherein the fragments of (a), (b), (c) and (d) elicit a human leukocyte antigen (HLA)-restricted CD8⁺ and/or CD4⁺ T cell response against a Plasmodium parasite, and are directly or indirectly fused in this order:
(a) a fragment of the inhibitor of cysteine protease (ICP) (18-10) of a Plasmodium parasite,
(b) a fragment of the protein Ag45 (11-10) of a Plasmodium parasite,
(c) a fragment of the thrombospondin related anonymous protein (TRAP) of a Plasmodium parasite, and
(d) the protein Ag40 (11-09) of a Plasmodium parasite or a fragment thereof, or a chimeric antigen variant thereof, which consists of a chimeric antigen having an amino acid sequence which has at least 90% sequence identity or more than 95% sequence identity or 99% sequence identity with the sequence of the fusion polypeptide consisting of fused fragments of (a), (b), (c) and (d), from which it derives by point mutation of one or more amino acid residues, over its whole length,
and wherein said process comprises:
1) co-transfecting helper cells, in particular HEK293 helper cells, that stably express T7 RNA polymerase, and measles N and P proteins with (i) the transfer vector plasmid according to claim 18 and with (ii) a vector, especially a plasmid, encoding the MV L polymerase;
2) cultivating said co-transfected helper cells in conditions enabling the production of recombinant MV-malaria virus particles;
3) propagating the thus produced recombinant MV-malaria virus particles by co-cultivating said helper cells of step 2) with cells enabling said propagation such as Vero cells;
4) recovering recombinant infectious replicating MV-malaria virus particles expressing (i) at least the CS protein of the Plasmodium parasite or the antigenic fragment thereof, and said chimeric antigen of the Plasmodium parasite, or (ii) at least the CS protein of the Plasmodium parasite or the antigenic fragment thereof, said chimeric antigen of the Plasmodium parasite and the RH5 of the Plasmodium parasite.

28. The process according to claim 27, wherein the transfer vector plasmid has the sequence of SEQ ID NO: 54 or SEQ ID NO: 55.


SEQUENCE LISTING (62 sequences)

SEQ ID NO: 1 - DNA, 75 bp, Artificial Sequence
  Description of Artificial Sequence Synthetic oligonucleotide
tctattatgggactgaaggtgaacgtgagcgcaatctttatggctgtgctgctgaccctgcagactcctaccggg

SEQ ID NO: 2 - protein, 25 aa, Artificial Sequence
  Description of Artificial Sequence Synthetic peptide
SIMGLKVNVSAIFMAVLLTLQTPTG

SEQ ID NO: 3 - DNA, 195 bp, Artificial Sequence
  Description of Artificial Sequence Synthetic polynucleotide
aaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctagtaa

SEQ ID NO: 4 - protein, 63 aa, Artificial Sequence
  Description of Artificial Sequence Synthetic polypeptide
KGLSSTSIVYILIAVCLGGLIGIPALICCCRGRCNKKGEQVGMSRPGLKPDLTGTSKSYVRSL

SEQ ID NO: 5 - DNA, 147 bp, Artificial Sequence
  Description of Artificial Sequence Synthetic polynucleotide
ttcgaactacagctcaacttacctgccaaccccatgccagtcgacccaactagtacaacctaaatccattataaaaaacttaggaaccaggtccacacagccgccagcccatcaaccatccactcccacgattggaggccggccatg

SEQ ID NO: 6 - protein, 20 aa, Artificial Sequence
  Description of Artificial Sequence Synthetic peptide
FELQLNLPANPMPVDPTSTT

SEQ ID NO: 7 - DNA, 115 bp, Artificial Sequence
  Description of Artificial Sequence Synthetic polynucleotide
cttaggaaccaggtccacacagccgccagcccatcaacgcgtacgatgtaggcgcgcagcgcttagacgtctcgcgatcgatactagtacaacctaaatccattataaaaaactt

SEQ ID NO: 8 - DNA, 1026 bp, Artificial Sequence
  Description of Artificial Sequence Synthetic polynucleotide
atggccaagaagtgtaccatactggtcgttgcgtcacttctgttggtcaattctctgctcccaggctatggacagaacaaatccattcaggcacagaggaacctcaacgaactctgctacaatgaagggaatgacaataagctgtatcacgtgctgaattccaagaacggcaaaatctacaatcgcaacacagtaaatcggttgcttgccgatgcacccgagggtaagaagaatgaaaagaagaatgagaaaatcgagcgcaacaacaagcttaaacagccaccgcctcctcctaacccaaatgacccaccgccacccaatccaaacgacccaccgcctcccaaccctaacgatcctccaccgcccaaccctaatgacccaccacctcccaatgcaaacgatccaccccctcctaacgctaacgaccctgctccacccaacgctaacgatcccgcgccccccaatgccaacgaccccgcaccacctaatgccaacgatcccgccccgcccaatgctaatgatcctccaccacccaacccaaacgaccctgcccctcctaatgctaacgatccaccacctcccaatccgaatgatcccgctccacctcaggggaacaacaaccctcagccccaacctagaccacagccgcagccccaaccccaaccccagccccagcctcaaccccagccccagccacgtccgcagcctcagcctcaacctggaggcaacaataacaacaagaataacaacaacgacgacagctacattccaagtgccgagaaaattctggagtttgttaagcagatccgagacagcataaccgaagaatggtcacagtgtaacgtgacgtgtggatctggcatcagagtgaggaaacggaagggttccaataagaaagcagaggatctgactctggaggacattgatacagagatctgcaaaatggacaaatgcagctctatcttcaacatcgtgagtaatagcctcgggtttgtgattctgctggtcctggtgttcttcaattga

SEQ ID NO: 9 - protein, 340 aa, Artificial Sequence
  Description of Artificial Sequence Synthetic polypeptide
AKKCTILVVASLLLVNSLLPGYGQNKSIQAQRNLNELCYNEGNDNKLYHVLNSKNGKIYNRNTVNRLLADAPEGKKNEKKNEKIERNNKLKQPPPPPNPNDPPPPNPNDPPPPNPNDPPPPNPNDPPPPNANDPPPPNANDPAPPNANDPAPPNANDPAPPNANDPAPPNANDPPPPNPNDPAPPNANDPPPPNPNDPAPPQGNNNPQPQPRPQPQPQPQPQPQPQPQPQPRPQPQPQPGGNNNNKNNNNDDSYIPSAEKILEFVKQIRDSITEEWSQCNVTCGSGIRVRKRKGSNKKAEDLTLEDIDTEICKMDKCSSIFNIVSNSLGFVILLVLVFFN

SEQ ID NO: 10 - DNA, 1137 bp, Artificial Sequence
  Description of Artificial Sequence Synthetic polynucleotide
atgtctattatgggactgaaggtgaacgtgagcgcaatctttatggctgtgctgctgaccctgcagactcctaccgggctgttccaggagtaccagtgttatgggtctagcagtaatactagggtgctgaacgaactgaattatgataacgcagggaccaatctgtacaacgagctggaaatgaattactacggaaagcaggagaactggtatagcctgaagaagaattcccgatctctgggcgagaacgatgatgggaataacgaggacaacgagaagctgaggaaacccaagcataagaagctgaagcagcccgccgatgggaacccagaccccaatgccaatcccaatgtggacccaaacgctaaccccaatgtcgatccaaatgccaacccaaatgtggacccaaatgcaaatcctaatgctaacccaaatgccaacccaaatgctaaccctaacgcaaatcccaatgcaaaccctaatgccaatcctaacgcaaatcctaacgcaaacccaaacgctaatccaaacgcaaatcctaacgctaatcctaacgctaatccaaacgccaatccaaacgccaatcctaacgccaatcccaacgcaaatccaaacgtggaccctaatgccaaccctaatgctaatccaaacgcaaaccctaatgcaaacccaaatgcaaaccccaatgcaaatcccaacgctaatcccaacgccaaccccaatgccaaccccaatgctaaccccaatgctaaccccaacgccaaccctaacgctaacccaaatgctaaccccaacgcaaaccccaacgccaacccaaacgccaacccaaatgcaaatcccaataagaacaatcagggtaatggacaggggcacaacatgccaaatgaccctaatcgaaacgtggacgagaacgccaatgcaaattcagctgtgaagaacaataataacgaagagccctctgataaacatatcaaggagtatctgaataagatccagaatagtctgtctaccgagtggtctccctgcagcgtgacatgtggaaatggaattcaggtccggattaagccagggtccgccaacaagcctaaggacgaactggactatgctaacgacattgagaagaagatttgctagtaa

SEQ ID NO: 11 - protein, 376 aa, Artificial Sequence
  Description of Artificial Sequence Synthetic polypeptide
SIMGLKVNVSAIFMAVLLTLQTPTGLFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKIC

SEQ ID NO: 12 - DNA, 1326 bp, Artificial Sequence
  Description of Artificial Sequence Synthetic polynucleotide
atgtctattatgggactgaaggtgaacgtgagcgcaatctttatggctgtgctgctgaccctgcagactcctaccgggctgttccaggagtaccagtgttatgggtctagcagtaatactagggtgctgaacgaactgaattatgataacgcagggaccaatctgtacaacgagctggaaatgaattactacggaaagcaggagaactggtatagcctgaagaagaattcccgatctctgggcgagaacgatgatgggaataacgaggacaacgagaagctgaggaaacccaagcataagaagctgaagcagcccgccgatgggaacccagaccccaatgccaatcccaatgtggacccaaacgctaaccccaatgtcgatccaaatgccaacccaaatgtggacccaaatgcaaatcctaatgctaacccaaatgccaacccaaatgctaaccctaacgcaaatcccaatgcaaaccctaatgccaatcctaacgcaaatcctaacgcaaacccaaacgctaatccaaacgcaaatcctaacgctaatcctaacgctaatccaaacgccaatccaaacgccaatcctaacgccaatcccaacgcaaatccaaacgtggaccctaatgccaaccctaatgctaatccaaacgcaaaccctaatgcaaacccaaatgcaaaccccaatgcaaatcccaacgctaatcccaacgccaaccccaatgccaaccccaatgctaaccccaatgctaaccccaacgccaaccctaacgctaacccaaatgctaaccccaacgcaaaccccaacgccaacccaaacgccaacccaaatgcaaatcccaataagaacaatcagggtaatggacaggggcacaacatgccaaatgaccctaatcgaaacgtggacgagaacgccaatgcaaattcagctgtgaagaacaataataacgaagagccctctgataaacatatcaaggagtatctgaataagatccagaatagtctgtctaccgagtggtctccctgcagcgtgacatgtggaaatggaattcaggtccggattaagccagggtccgccaacaagcctaaggacgaactggactatgctaacgacattgagaagaagatttgcaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctagtaa

SEQ ID NO: 13 - protein, 439 aa, Artificial Sequence
  Description of Artificial Sequence Synthetic polypeptide
SIMGLKVNVSAIFMAVLLTLQTPTGLFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKGLSSTSIVYILIAVCLGGLIGIPALICCCRGRCNKKGEQVGMSRPGLKPDLTGTSKSYVRSL

SEQ ID NO: 14 - DNA, 348 bp, Artificial Sequence
  Description of Artificial Sequence Synthetic polynucleotide
gacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattcc

SEQ ID NO: 15 - protein, 116 aa, Artificial Sequence
  Description of Artificial Sequence Synthetic polypeptide
DNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNS

SEQ ID NO: 16 - DNA, 501 bp, Artificial Sequence
  Description of Artificial Sequence Synthetic polynucleotide
ctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacag

SEQ ID NO: 17 - protein, 167 aa, Artificial Sequence
  Description of Artificial Sequence Synthetic polypeptide
LTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQ

SEQ ID NO: 18 - DNA, 1173 bp, Artificial Sequence
  Description of Artificial Sequence Synthetic polynucleotide
gacaacaacagctacagcttcgagatcgtgaaccgcagcacctggctgaacatcgccgagcgcatcttcaagggcaacgcccccttcaacttcaccatcatcccctacaactacgtgaacaacagcaccgaggagaacaacaacaaggacagcgtgctgctgatcagcaagaacctgaagaacagcagcaaccccgtggacgagaacaaccacatcatcgacagcaccaagaagaacaccagcaacaacaacaacaacaacagcaacatcgtgggcatctacgagagccaggtgcacgaggagaagatcaaggaggacaacacccgccaggacaacatcaacaagaaggagaacgagatcatcaacaacaaccaccagatccccgtgagcaacatcttcagcgagaacatcgacaacaacaagaactacatcgagagcaactacaagagcacctacaacaacaaccccgagctgatccacagcaccgacttcatcggcagcaacaacaaccacaccttcaacttcctgagccgctacaacaacagcgtgctgaacaacatgcagggcaacaccaaggtgcccggcaacgtgcccgagctgaaggcccgcatcttcagcgaggaggagaacaccgaggtggagagcgccgagaacaaccacaccaacagcctgaaccccaacgagagctgcgaccagatcatcaagctgggcgacatcatcaacagcgtgaacgagaagatcatcagcatcaacagcaccgtgaacaacgtgctgtgcatcaacctggacagcgtgaacggcaacggcttcgtgtggaccctgctgggcgtgcacaagaagaagcccctgatcgaccccagcaacttccccaccaagcgcgtgacccagagctacgtgagccccgacatcagcgtgaccaaccccgtgcccatccccaagaacagcaacaccaacaaggacgacagcatcaacaacaagcaggacggcagccagaacaacaccaccaccaaccacttccccaagccccgcgagcagctggtgggcggcagcagcatgctgatcagcaagatcaagccccacaagcccggcaagtacttcatcgtgtacagctactaccgccccttcgaccccacccgcgacaccaacacccgcatcgtggagctgaacgtgcag

SEQ ID NO: 19 - protein, 391 aa, Artificial Sequence
  Description of Artificial Sequence Synthetic polypeptide
DNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQ

SEQ ID NO: 20 - DNA, 501 bp, Artificial Sequence
  Description of Artificial Sequence Synthetic polynucleotide
ctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaa

SEQ ID NO: 21 - protein, 167 aa, Artificial Sequence
  Description of Artificial Sequence Synthetic polypeptide
LIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKK

SEQ ID NO: 22 - DNA, 537 bp, Artificial Sequence
  Description of Artificial Sequence Synthetic polynucleotide
gaggaggacgacaaggaggtggagcccgaggaggagaaggagaacgagcagaagaaggaggagcaggaggagaacaacctggaggcccccagcaagaccctgatgaagggcgtgaagaccaacatctacttcctgagcaccaaggagcgcatcgaggccctgatgtgctacaactacatcagcaacgccatcatcttcgagaagggcaagttcctgcgctacatcttcatgaacaacgtgaacaacatcatcgtgaacgagcacatgatcaacatgctgtgcaagaaggagaagatcaagtacatcctgagcagcaacagcatcatcatcgagagcaacgacttcatcaagcccctgatcatcgagttcgacagcaacatcagcaagaagatcttcgtgaagcacctgaagatggtggacagcttcaagctggacgacaagctgtaccgcgagtacctgaacgacctgagcgagcacgagcgcgaccgcctgaagcacgtggagagcttctacagcaacgccatcaaggtgcacaac

SEQ ID NO: 23 - protein, 179 aa, Artificial Sequence
  Description of Artificial Sequence Synthetic polypeptide
EEDDKEVEPEEEKENEQKKEEQEENNLEAPSKTLMKGVKTNIYFLSTKERIEALMCYNYISNAIIFEKGKFLRYIFMNNVNNIIVNEHMINMLCKKEKIKYILSSNSIIIESNDFIKPLIIEFDSNISKKIFVKHLKMVDSFKLDDKLYREYLNDLSEHERDRLKHVESFYSNAIKVHN

SEQ ID NO: 24 - DNA, 714 bp, Artificial Sequence
  Description of Artificial Sequence Synthetic polynucleotide
caggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggtt

SEQ ID NO: 25 - protein, 238 aa, Artificial Sequence
  Description of Artificial Sequence Synthetic polypeptide
QEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKV

SEQ ID NO: 26 - DNA, 879 bp, Artificial Sequence
  Description of Artificial Sequence Synthetic polynucleotide
gacgtgcagaacaacatcgtggacgagatcaagtaccgcgaggaggtgtgcaacgacgaggtggacctgtacctgctgatggactgcagcggcagcatccgccgccacaactgggtgaaccacgccgtgcccctggccatgaagctgatccagcagctgaacctgaacgacaacgccatccacctgtacgccagcgtgttcagcaacaacgcccgcgagatcatccgcctgcacagcgacgccagcaagaacaaggagaaggccctgatcatcatcaagagcctgctgagcaccaacctgccctacggcaagaccaacctgaccgacgccctgctgcaggtgcgcaagcacctgaacgaccgcatcaaccgcgagaacgccaaccagctggtggtgatcctgaccgacggcatccccgacagcatccaggacagcctgaaggagagccgcaagctgagcgaccgcggcgtgaagatcgccgtgttcggcatcggccagggcatcaacgtggccttcaaccgcttcctggtgggctgccaccccagcgacggcaagtgcaacctgtacgccgacagcgcctgggagaacgtgaagaacgtgatcggccccttcatgaaggccgtgtgcgtggaggtggagaagaccgccagctgcggcgtgtgggacgagtggagcccctgcagcgtgacctgcggcaagggcacccgcagccgcaagcgcgagatcctgcacgagggctgcaccagcgagctgcaggagcagtgcgaggaggagcgctgcctgcccaagcgcgagcccctggacgtgcccgacgagcccgaggacgaccagccccgcccccgcggcgacaacttcgccgtggagaagcccaacgagaac

SEQ ID NO: 27 - protein, 293 aa, Artificial Sequence
  Description of Artificial Sequence Synthetic polypeptide
DVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCLPKREPLDVPDEPEDDQPRPRGDNFAVEKPNEN

SEQ ID NO: 28 - DNA, 630 bp, Artificial Sequence
  Description of Artificial Sequence Synthetic polynucleotide
ggggcgagtatgagccacttgcagtgtctgacatctgttgctggtctgtcctctatcgtcatgtcaatgttccccaaactcattgccaataatccttccctgtttagaccactgctcaacatttcctggggatatctgttcggaagcactgtatggctgtgcttcttcagtgagattgggttggtcaggagaatcaatgctcctaaacggaagaatctgccagagaatgcagaacaagccaaagaacagctgaaggagatcaagaacaacgaaggcgattttaaccgacgcaatatcgacttcaagtacttctttagcctttccacaatcttctctagcatactgctgcttagcacagtgaaactcgccaacaacaatctgcagttgaggatctgttccaccattgtgtcactgagttgcatactgaacaatatgtactttcagaacaagatacactcacttgcactgaagaaagagagtctctttaaggacatgatcgatcgtccgaaagataccactattctggtgaacctgaagaagaacaagaccgactttcacatccatcatggcctttctctgctcttgctctatagcagcttctttggcctcactccctacattttcacgtga

SEQ ID NO: 29 - protein, 209 aa, Artificial Sequence
  Description of Artificial Sequence Synthetic polypeptide
GASMSHLQCLTSVAGLSSIVMSMFPKLIANNPSLFRPLLNISWGYLFGSTVWLCFFSEIGLVRRINAPKRKNLPENAEQAKEQLKEIKNNEGDFNRRNIDFKYFFSLSTIFSSILLLSTVKLANNNLQLRICSTIVSLSCILNNMYFQNKIHSLALKKESLFKDMIDRPKDTTILVNLKKNKTDFHIHHGLSLLLLYSSFFGLTPYIFT

SEQ ID NO: 30 - DNA, 615 bp, Artificial Sequence
  Description of Artificial Sequence Synthetic polynucleotide
agcaacctgaagtgcgtgaccaacgtggccggcctggccagcctggtgatcagcctgttccccaagctgatcatcaagaacccccaggtgctgcgccccctgctgaacgtgagctggggctacctgttcggcagcaccttctggctgtgcttcttcagcgaggtgggcctgctgcgcagcctgaagaacatgaagggcgtgcccctgcccgagagcgccagcgaggccaagaagctgctggaggagatgaagaacagcgagggcgacttcaaccgccgcagcctggacttccagtacttcttcagcctggccaccctgttcagcggcatcctgctgctgagcaccgtgaagctggccaaccacaacctgcagctgcgcctgagcagcagcgtggtggtgatcaccagcctgctgaacagcctgtacctgcacaacaaggtgcacaacctgaagagcaagaaggagagcctgtacaacgacttcatcgccaaccccaagaacgagaagaccgtggccgacctgaagaagaacaagaaggagttccacatcttccacggcctgagcgtgctgagcctgtacgtgagcttcttcggcctgaccccctacatcttcacc

SEQ ID NO: 31 - protein, 205 aa, Artificial Sequence
  Description of Artificial Sequence Synthetic polypeptide
SNLKCVTNVAGLASLVISLFPKLIIKNPQVLRPLLNVSWGYLFGSTFWLCFFSEVGLLRSLKNMKGVPLPESASEAKKLLEEMKNSEGDFNRRSLDFQYFFSLATLFSGILLLSTVKLANHNLQLRLSSSVVVITSLLNSLYLHNKVHNLKSKKESLYNDFIANPKNEKTVADLKKNKKEFHIFHGLSVLSLYVSFFGLTPYIFT

SEQ ID NO: 32 - DNA, 1581 bp, Artificial Sequence
  Description of Artificial Sequence Synthetic polynucleotide
atgtctattatgggactgaaggtgaacgtgagcgcaatctttatggctgtgctgctgaccctgcagactcctaccggggaaaatgcaataaaaaaaacgaagaatcaagaaaataatctgacgttactaccaataaagagcactgaagaagaaaaagatgatataaaaaatggaaaggatataaaaaaagaaattgataatgataaagagaatataaaaacaaataatgctaaagatcattcaacatatataaaatcatatttgaatacaaatgtaaatgatggtttaaaatatttgtttattccttctcataattcttttataaaaaaatattctgtatttaatcaaataaatgatggcatgttattaaatgaaaaaaatgatgtgaaaaataatgaagactataaaaatgtggattataaaaatgttaattttttacaatatcattttaaagagttatcaaattataacattgcaaattctattgatattttacaagaaaaagaaggacatttggattttgttataatacctcattatacttttttagattattataaacatttatcttataattctatatatcataagtcctctacatatggaaagtgtatagctgtagatgcttttattaagaaaataaatgaaacatatgacaaagtgaaaagtaaatgtaatgatataaagaatgatttaattgcaactataaaaaaattagagcatccttatgatataaataataagaatgatgattcctatagatatgatatatctgaagaaatcgatgataaatctgaagagacagatgatgaaaccgaagaggtagaagatagtatacaagatacagatagtaatcatactccttcaaataaaaaaaaaaatgatcttatgaatagaacgtttaaaaagatgatggatgaatataatacaaaaaaaaaaaaattaattaaatgtataaaaaaccatgagaatgattttaataaaatatgtatggatatgaaaaattatggtacaaacctttttgaacaactttcatgttacaataataatttctgtaatacaaacggaataagatatcattatgatgaatatattcataaattaatattatctgttaaatcaaaaaacttaaataaagacctatcagatatgacaaatattttacaacaaagtgaattattattaaccaatttaaataaaaaaatgggttcctatatatatattgatacaataaaatttatacataaagaaatgaaacatatttttaacagaattgaatatcatacaaaaataataaacgataaaactaaaataattcaagacaaaattaaattaaatatatggagaacatttcaaaaagatgaattattaaaaagaattttagacatgtcaaatgaatattctttatttattactagtgatcatttaagacaaatgttatataatacattctattcaaaagaaaaacatttaaataatatatttcatcatttaatttatgtactacaaatgaagttcaatgatgtcccaattaaaatggaatattttcaaacatataaaaaaaataaaccacttacacaa

SEQ ID NO: 33 - protein, 525 aa, Artificial Sequence
  Description of Artificial Sequence Synthetic polypeptide
IRIKKKLILTIIYIHLFILNRLSFENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 34 - DNA, 1776 bp, Artificial Sequence
  Description of Artificial Sequence Synthetic polynucleotide
atgtctattatgggactgaaggtgaacgtgagcgcaatctttatggctgtgctgctgaccctgcagactcctaccggggaaaatgcaataaaaaaaacgaagaatcaagaaaataatctgacgttactaccaataaagagcactgaagaagaaaaagatgatataaaaaatggaaaggatataaaaaaagaaattgataatgataaagagaatataaaaacaaataatgctaaagatcattcaacatatataaaatcatatttgaatacaaatgtaaatgatggtttaaaatatttgtttattccttctcataattcttttataaaaaaatattctgtatttaatcaaataaatgatggcatgttattaaatgaaaaaaatgatgtgaaaaataatgaagactataaaaatgtggattataaaaatgttaattttttacaatatcattttaaagagttatcaaattataacattgcaaattctattgatattttacaagaaaaagaaggacatttggattttgttataatacctcattatacttttttagattattataaacatttatcttataattctatatatcataagtcctctacatatggaaagtgtatagctgtagatgcttttattaagaaaataaatgaaacatatgacaaagtgaaaagtaaatgtaatgatataaagaatgatttaattgcaactataaaaaaattagagcatccttatgatataaataataagaatgatgattcctatagatatgatatatctgaagaaatcgatgataaatctgaagagacagatgatgaaaccgaagaggtagaagatagtatacaagatacagatagtaatcatactccttcaaataaaaaaaaaaatgatcttatgaatagaacgtttaaaaagatgatggatgaatataatacaaaaaaaaaaaaattaattaaatgtataaaaaaccatgagaatgattttaataaaatatgtatggatatgaaaaattatggtacaaacctttttgaacaactttcatgttacaataataatttctgtaatacaaacggaataagatatcattatgatgaatatattcataaattaatattatctgttaaatcaaaaaacttaaataaagacctatcagatatgacaaatattttacaacaaagtgaattattattaaccaatttaaataaaaaaatgggttcctatatatatattgatacaataaaatttatacataaagaaatgaaacatatttttaacagaattgaatatcatacaaaaataataaacgataaaactaaaataattcaagacaaaattaaattaaatatatggagaacatttcaaaaagatgaattattaaaaagaattttagacatgtcaaatgaatattctttatttattactagtgatcatttaagacaaatgttatataatacattctattcaaaagaaaaacatttaaataatatatttcatcatttaatttatgtactacaaatgaagttcaatgatgtcccaattaaaatggaatattttcaaacatataaaaaaaataaaccacttacacaaaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctagtga

SEQ ID NO: 35 - protein, 589 aa, Artificial Sequence
  Description of Artificial Sequence Synthetic polypeptide
SIMGLKVNVSAIFMAVLLTLQTPTGENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQKGLSSTSIVYILIAVCLGGLIGIPALICCCRGRCNKKGEQVGMSRPGLKPDLTGTSKSYVRSL

SEQ ID NO: 36 - DNA, 2862 bp, Artificial Sequence
  Description of Artificial Sequence Synthetic polynucleotide
atgtcaatcatgggactgaaggtgaacgtcagcgcaatcttcatggccgtgctgctgactctgcagaccccaacaggcgaaaatgcaataaaaaaaacgaagaatcaagaaaataatctgacgttactaccaataaagagcactgaagaagaaaaagatgatataaaaaatggaaaggatataaaaaaagaaattgataatgataaagagaatataaaaacaaataatgctaaagatcattcaacatatataaaatcatatttgaatacaaatgtaaatgatggtttaaaatatttgtttattccttctcataattcttttataaaaaaatattctgtatttaatcaaataaatgatggcatgttattaaatgaaaaaaatgatgtgaaaaataatgaagactataaaaatgtggattataaaaatgttaattttttacaatatcattttaaagagttatcaaattataacattgcaaattctattgatattttacaagaaaaagaaggacatttggattttgttataatacctcattatacttttttagattattataaacatttatcttataattctatatatcataagtcctctacatatggaaagtgtatagctgtagatgcttttattaagaaaataaatgaaacatatgacaaagtgaaaagtaaatgtaatgatataaagaatgatttaattgcaactataaaaaaattagagcatccttatgatataaataataagaatgatgattcctatagatatgatatatctgaagaaatcgatgataaatctgaagagacagatgatgaaaccgaagaggtagaagatagtatacaagatacagatagtaatcatactccttcaaataaaaaaaaaaatgatcttatgaatagaacgtttaaaaagatgatggatgaatataatacaaaaaaaaaaaaattaattaaatgtataaaaaaccatgagaatgattttaataaaatatgtatggatatgaaaaattatggtacaaacctttttgaacaactttcatgttacaataataatttctgtaatacaaacggaataagatatcattatgatgaatatattcataaattaatattatctgttaaatcaaaaaacttaaataaagacctatcagatatgacaaatattttacaacaaagtgaattattattaaccaatttaaataaaaaaatgggttcctatatatatattgatacaataaaatttatacataaagaaatgaaacatatttttaacagaattgaatatcatacaaaaataataaacgataaaactaaaataattcaagacaaaattaaattaaatatatggagaacatttcaaaaagatgaattattaaaaagaattttagacatgtcaaatgaatattctttatttattactagtgatcatttaagacaaatgttatataatacattctattcaaaagaaaaacatttaaataatatatttcatcatttaatttatgtactacaaatgaagttcaatgatgtcccaattaaaatggaatattttcaaacatataaaaaaaataaaccacttacacaatgattcgaactacagctcaacttacctgccaaccccatgccagtcgacccaactagtacaacctaaatccattataaaaaacttaggaaccaggtccacacagccgccagcccatcaaccatccactcccacgattggaggccggccatgtctattatgggactgaaggtgaacgtgagcgcaatctttatggctgtgctgctgaccctgcagactcctaccgggctgttccaggagtaccagtgttatgggtctagcagtaatactagggtgctgaacgaactgaattatgataacgcagggaccaatctgtacaacgagctggaaatgaattactacggaaagcaggagaactggtatagcctgaagaagaattcccgatctctgggcgagaacgatgatgggaataacgaggacaacgagaagctgaggaaacccaagcataagaagctgaagcagcccgccgatgggaacccagaccccaatgccaatcccaatgtggacccaaacgctaaccccaatgtcgatccaaatgccaacccaaatgtggacccaaatgcaaatcctaatgctaacccaaatgccaacccaaatgctaaccctaacgcaaatcccaatgcaaaccctaatgccaatcctaacgcaaatcctaacgcaaacccaaacgctaatccaaacgcaaatcctaacgctaatcctaacgctaatccaaacgccaatccaaacgccaatcctaacgccaatcccaacgcaaatccaaacgtggaccctaatgccaaccctaatgctaatccaaacgcaaaccctaatgcaaacccaaatgcaaaccccaatgcaaatcccaacgctaatcccaacgccaaccccaatgccaaccccaatgctaaccccaatgctaaccccaacgccaaccctaacgctaacccaaatgctaaccccaacgcaaaccccaacgccaacccaaacgccaacccaaatgcaaatcccaataagaacaatcagggtaatggacaggggcacaacatgccaaatgaccctaatcgaaacgtggacgagaacgccaatgcaaattcagctgtgaagaacaataataacgaagagccctctgataaacatatcaaggagtatctgaataagatccagaatagtctgtctaccgagtggtctccctgcagcgtgacatgtggaaatggaattcaggtccggattaagccagggtccgccaacaagcctaaggacgaactggactatgctaacgacattgagaagaagatttgctag

SEQ ID NO: 37 - protein, 526 aa, Artificial Sequence
  Description of Artificial Sequence Synthetic polypeptide
SIMGLKVNVSAIFMAVLLTLQTPTGENAIKKTKNQENNLTLLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNDDSYRYDISEEIDDKSEETDDETEEVEDSIQDTDSNHTPSNKKKNDLMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 38 - DNA, 3246 bp, Artificial Sequence
  Description of Artificial Sequence Synthetic polynucleotide
atgtctattatgggactgaaggtgaacgtgagcgcaatctttatggctgtgctgctgaccctgcagactcctaccggggaaaatgcaataaaaaaaacgaagaatcaagaaaataatctgacgttactaccaataaagagcactgaagaagaaaaagatgatataaaaaatggaaaggatataaaaaaagaaattgataatgataaagagaatataaaaacaaataatgctaaagatcattcaacatatataaaatcatatttgaatacaaatgtaaatgatggtttaaaatatttgtttattccttctcataattcttttataaaaaaatattctgtatttaatcaaataaatgatggcatgttattaaatgaaaaaaatgatgtgaaaaataatgaagactataaaaatgtggattataaaaatgttaattttttacaatatcattttaaagagttatcaaattataacattgcaaattctattgatattttacaagaaaaagaaggacatttggattttgttataatacctcattatacttttttagattattataaacatttatcttataattctatatatcataagtcctctacatatggaaagtgtatagctgtagatgcttttattaagaaaataaatgaaacatatgacaaagtgaaaagtaaatgtaatgatataaagaatgatttaattgcaactataaaaaaattagagcatccttatgatataaataataagaatgatgattcctatagatatgatatatctgaagaaatcgatgataaatctgaagagacagatgatgaaaccgaagaggtagaagatagtatacaagatacagatagtaatcatactccttcaaataaaaaaaaaaatgatcttatgaatagaacgtttaaaaagatgatggatgaatataatacaaaaaaaaaaaaattaattaaatgtataaaaaaccatgagaatgattttaataaaatatgtatggatatgaaaaattatggtacaaacctttttgaacaactttcatgttacaataataatttctgtaatacaaacggaataagatatcattatgatgaatatattcataaattaatattatctgttaaatcaaaaaacttaaataaagacctatcagatatgacaaatattttacaacaaagtgaattattattaaccaatttaaataaaaaaatgggttcctatatatatattgatacaataaaatttatacataaagaaatgaaacatatttttaacagaattgaatatcatacaaaaataataaacgataaaactaaaataattcaagacaaaattaaattaaatatatggagaacatttcaaaaagatgaattattaaaaagaattttagacatgtcaaatgaatattctttatttattactagtgatcatttaagacaaatgttatataatacattctattcaaaagaaaaacatttaaataatatatttcatcatttaatttatgtactacaaatgaagttcaatgatgtcccaattaaaatggaatattttcaaacatataaaaaaaataaaccacttacacaaaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctagtgattcgaactacagctcaacttacctgccaaccccatgccagtcgacccaactagtacaacctaaatccattataaaaaacttaggaaccaggtccacacagccgccagcccatcaaccatccactcccacgattggaggccggccatgtctattatgggactgaaggtgaacgtgagcgcaatctttatggctgtgctgctgaccctgcagactcctaccgggctgttccaggagtaccagtgttatgggtctagcagtaatactagggtgctgaacgaactgaattatgataacgcagggaccaatctgtacaacgagctggaaatgaattactacggaaagcaggagaactggtatagcctgaagaagaattcccgatctctgggcgagaacgatgatgggaataacgaggacaacgagaagctgaggaaacccaagcataagaagctgaagcagcccgccgatgggaacccagaccccaatgccaatcccaatgtggacccaaacgctaaccccaatgtcgatccaaatgccaacccaaatgtggacccaaatgcaaatcctaatgctaacccaaatgccaacccaaatgctaaccctaacgcaaatcccaatgcaaaccctaatgccaatcctaacgcaaatcctaacgcaaacccaaacgctaatccaaacgcaaatcctaacgctaatcctaacgctaatccaaacgccaatccaaacgccaatcctaacgccaatcccaacgcaaatccaaacgtggaccctaatgccaaccctaatgctaatccaaacgcaaaccctaatgcaaacccaaatgcaaaccccaatgcaaatcccaacgctaatcccaacgccaaccccaatgccaaccccaatgctaaccccaatgctaaccccaacgccaaccctaacgctaacccaaatgctaaccccaacgcaaaccccaacgccaacccaaacgccaacccaaatgcaaatcccaataagaacaatcagggtaatggacaggggcacaacatgccaaatgaccctaatcgaaacgtggacgagaacgccaatgcaaattcagctgtgaagaacaataataacgaagagccctctgataaacatatcaaggagtatctgaataagatccagaatagtctgtctaccgagtggtctccctgcagcgtgacatgtggaaatggaattcaggtccggattaagccagggtccgccaacaagcctaaggacgaactggactatgctaacgacattgagaagaagatttgcaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctagtaa

SEQ ID NO: 39 - protein, 589 aa, Artificial Sequence; identical to SEQ ID NO: 35
  Description of Artificial Sequence Synthetic polypeptide

SEQ ID NO: 40 - DNA, 2772 bp, Artificial Sequence
  Description of Artificial Sequence Synthetic polynucleotide
atgtctattatgggactgaaggtgaacgtgagcgcaatctttatggctgtgctgctgaccctgcagactcctaccggggacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattccctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacagctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaacaggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggttggggcgagtatgagccacttgcagtgtctgacatctgttgctggtctgtcctctatcgtcatgtcaatgttccccaaactcattgccaataatccttccctgtttagaccactgctcaacatttcctggggatatctgttcggaagcactgtatggctgtgcttcttcagtgagattgggttggtcaggagaatcaatgctcctaaacggaagaatctgccagagaatgcagaacaagccaaagaacagctgaaggagatcaagaacaacgaaggcgattttaaccgacgcaatatcgacttcaagtacttctttagcctttccacaatcttctctagcatactgctgcttagcacagtgaaactcgccaacaacaatctgcagttgaggatctgttccaccattgtgtcactgagttgcatactgaacaatatgtactttcagaacaagatacactcacttgcactgaagaaagagagtctctttaaggacatgatcgatcgtccgaaagataccactattctggtgaacctgaagaagaacaagaccgactttcacatccatcatggcctttctctgctcttgctctatagcagcttctttggcctcactccctacattttcacgtga

SEQ ID NO: 41 - protein, 922 aa, Artificial Sequence
  Description of Artificial Sequence Synthetic polypeptide
SIMGLKVNVSAIFMAVLLTLQTPTGDNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNSLTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQLIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKKQEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKVGASMSHLQCLTSVAGLSSIVMSMFPKLIANNPSLFRPLLNISWGYLFGSTVWLCFFSEIGLVRRINAPKRKNLPENAEQAKEQLKEIKNNEGDFNRRNIDFKYFFSLSTIFSSILLLSTVKLANNNLQLRICSTIVSLSCILNNMYFQNKIHSLALKKESLFKDMIDRPKDTTILVNLKKNKTDFHIHHGLSLLLLYSSFFGLTPYIFT

SEQ ID NO: 42 - DNA, 2694 bp, Artificial Sequence
  Description of Artificial Sequence Synthetic polynucleotide
gacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattccctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacagctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaacaggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggttggggcgagtatgagccacttgcagtgtctgacatctgttgctggtctgtcctctatcgtcatgtcaatgttccccaaactcattgccaataatccttccctgtttagaccactgctcaacatttcctggggatatctgttcggaagcactgtatggctgtgcttcttcagtgagattgggttggtcaggagaatcaatgctcctaaacggaagaatctgccagagaatgcagaacaagccaaagaacagctgaaggagatcaagaacaacgaaggcgattttaaccgacgcaatatcgacttcaagtacttctttagcctttccacaatcttctctagcatactgctgcttagcacagtgaaactcgccaacaacaatctgcagttgaggatctgttccaccattgtgtcactgagttgcatactgaacaatatgtactttcagaacaagatacactcacttgcactgaagaaagagagtctctttaaggacatgatcgatcgtccgaaagataccactattctggtgaacctgaagaagaacaagaccgactttcacatccatcatggcctttctctgctcttgctctatagcagcttctttggcctcactccctacattttcacgtga

SEQ ID NO: 43 - protein, 897 aa, Artificial Sequence
  Description of Artificial Sequence Synthetic polypeptide
DNDIYSFDIVNETNWLKIAKNIFKGKSPSNFTIIPFNNTGSSNDNESNKEESVLLIRKKIKSNKNHDSSIISGDTVNGDISDLNYTASNFSDNSEDIEDNQKYPTTSYNSFNHLNSLTGDEKCGKSLKLGNISNQTNQETITQSLSVGEILCIDLEGNAGTGYLWVLLGIHKDEPIINPENFPTKLTKKSFFSEEISVTQPKKYKIDEHDSSKNVNREIESPEQKESDSKPKKPQMQLLGGPDRMRSVIKGHKPGKYYIVYSYYRPFSPTSGANTKIIYVTVQLIEEKEEITPENKTLIMPSKTLMKGIKTNIYFLSNKEKIQVLMCYNYKCDAVVFEKDTFLRYLYIKSINNIILNERMIEQLCKNENLKYILACNSIVVESSDFIKPLIIEFESSTSKNIFVKHIKHNSQKEMDINKFNEYMRDLKSNEKLRLKKVERFHSINLAAKKQEILDEIKYSEEVCNEQIDLHILLDGSGSIGHSNWISHVIPMLTTLVDNLNISRDEINISMTLFSTYARELVRLKRYGSTSKASLRFIIAQLQNNYSPHGTTNLTSALLNVDNLIQKKMNRPNAIQLVIILTDGIPNNLKKSTTVVNQLKKKDVNVAIIGVGAGVNNMFNRILVGCGKLGPCPYYSYGSWDQAQTMIKPFLSKVCQEVEKVALCGKWEEWSECSTTCDNGTKIRKRKVGASMSHLQCLTSVAGLSSIVMSMFPKLIANNPSLFRPLLNISWGYLFGSTVWLCFFSEIGLVRRINAPKRKNLPENAEQAKEQLKEIKNNEGDFNRRNIDFKYFFSLSTIFSSILLLSTVKLANNNLQLRICSTIVSLSCILNNMYFQNKIHSLALKKESLFKDMIDRPKDTTILVNLKKNKTDFHIHHGLSLLLLYSSFFGLTPYIFT

SEQ ID NO: 44 - DNA, 3288 bp, Artificial Sequence
  Description of Artificial Sequence Synthetic polynucleotide
atgtctattatgggactgaaggtgaacgtgagcgcaatctttatggctgtgctgctgaccctgcagactcctaccggggacaacaacagctacagcttcgagatcgtgaaccgcagcacctggctgaacatcgccgagcgcatcttcaagggcaacgcccccttcaacttcaccatcatcccctacaactacgtgaacaacagcaccgaggagaacaacaacaaggacagcgtgctgctgatcagcaagaacctgaagaacagcagcaaccccgtggacgagaacaaccacatcatcgacagcaccaagaagaacaccagcaacaacaacaacaacaacagcaacatcgtgggcatctacgagagccaggtgcacgaggagaagatcaaggaggacaacacccgccaggacaacatcaacaagaaggagaacgagatcatcaacaacaaccaccagatccccgtgagcaacatcttcagcgagaacatcgacaacaacaagaactacatcgagagcaactacaagagcacctacaacaacaaccccgagctgatccacagcaccgacttcatcggcagcaacaacaaccacaccttcaacttcctgagccgctacaacaacagcgtgctgaacaacatgcagggcaacaccaaggtgcccggcaacgtgcccgagctgaaggcccgcatcttcagcgaggaggagaacaccgaggtggagagcgccgagaacaaccacaccaacagcctgaaccccaacgagagctgcgaccagatcatcaagctgggcgacatcatcaacagcgtgaacgagaagatcatcagcatcaacagcaccgtgaacaacgtgctgtgcatcaacctggacagcgtgaacggcaacggcttcgtgtggaccctgctgggcgtgcacaagaagaagcccctgatcgaccccagcaacttccccaccaagcgcgtgacccagagctacgtgagccccgacatcagcgtgaccaaccccgtgcccatccccaagaacagcaacaccaacaaggacgacagcatcaacaacaagcaggacggcagccagaacaacaccaccaccaaccacttccccaagccccgcgagcagctggtgggcggcagcagcatgctgatcagcaagatcaagccccacaagcccggcaagtacttcatcgtgtacagctactaccgccccttcgaccccacccgcgacaccaacacccgcatcgtggagctgaacgtgcaggaggaggacgacaaggaggtggagcccgaggaggagaaggagaacgagcagaagaaggaggagcaggaggagaacaacctggaggcccccagcaagaccctgatgaagggcgtgaagaccaacatctacttcctgagcaccaaggagcgcatcgaggccctgatgtgctacaactacatcagcaacgccatcatcttcgagaagggcaagttcctgcgctacatcttcatgaacaacgtgaacaacatcatcgtgaacgagcacatgatcaacatgctgtgcaagaaggagaagatcaagtacatcctgagcagcaacagcatcatcatcgagagcaacgacttcatcaagcccctgatcatcgagttcgacagcaacatcagcaagaagatcttcgtgaagcacctgaagatggtggacagcttcaagctggacgacaagctgtaccgcgagtacctgaacgacctgagcgagcacgagcgcgaccgcctgaagcacgtggagagcttctacagcaacgccatcaaggtgcacaacgaggacgtgcagaacaacatcgtggacgagatcaagtaccgcgaggaggtgtgcaacgacgaggtggacctgtacctgctgatggactgcagcggcagcatccgccgccacaactgggtgaaccacgccgtgcccctggccatgaagctgatccagcagctgaacctgaacgacaacgccatccacctgtacgccagcgtgttcagcaacaacgcccgcgagatcatccgcctgcacagcgacgccagcaagaacaaggagaaggccctgatcatcatcaagagcctgctgagcaccaacctgccctacggcaagaccaacctgaccgacgccctgctgcaggtgcgcaagcacctgaacgaccgcatcaaccgcgagaacgccaaccagctggtggtgatcctgaccgacggcatccccgacagcatccaggacagcctgaaggagagccgcaagctgagcgaccgcggcgtgaagatcgccgtgttcggcatcggccagggcatcaacgtggccttcaaccgcttcctggtgggctgccaccccagcgacggcaagtgcaacctgtacgccgacagcgcctgggagaacgtgaagaacgtgatcggccccttcatgaaggccgtgtgcgtggaggtggagaagaccgccagctgcggcgtgtgggacgagtggagcccctgcagcgtgacctgcggcaagggcacccgcagccgcaagcgcgagatcctgcacgagggctgcaccagcgagctgcaggagcagtgcgaggaggagcgctgcctgcccaagcgcgagcccctggacgtgcccgacgagcccgaggacgaccagccccgcccccgcggcgacaacttcgccgtggagaagcccaacgagaacagcaacctgaagtgcgtgaccaacgtggccggcctggccagcctggtgatcagcctgttccccaagctgatcatcaagaacccccaggtgctgcgccccctgctgaacgtgagctggggctacctgttcggcagcaccttctggctgtgcttcttcagcgaggtgggcctgctgcgcagcctgaagaacatgaagggcgtgcccctgcccgagagcgccagcgaggccaagaagctgctggaggagatgaagaacagcgagggcgacttcaaccgccgcagcctggacttccagtacttcttcagcctggccaccctgttcagcggcatcctgctgctgagcaccgtgaagctggccaaccacaacctgcagctgcgcctgagcagcagcgtggtggtgatcaccagcctgctgaacagcctgtacctgcacaacaaggtgcacaacctgaagagcaagaaggagagcctgtacaacgacttcatcgccaaccccaagaacgagaagaccgtggccgacctgaagaagaacaagaaggagttccacatcttccacggcctgagcgtgctgagcctgtacgtgagcttcttcggcctgaccccctacatcttcacctga

SEQ ID NO: 45 - protein, 1095 aa, Artificial Sequence
  Description of Artificial Sequence Synthetic polypeptide
MSIMGLKVNVSAIFMAVLLTLQTPTGDNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQEEDDKEVEPEEEKENEQKKEEQEENNLEAPSKTLMKGVKTNIYFLSTKERIEALMCYNYISNAIIFEKGKFLRYIFMNNVNNIIVNEHMINMLCKKEKIKYILSSNSIIIESNDFIKPLIIEFDSNISKKIFVKHLKMVDSFKLDDKLYREYLNDLSEHERDRLKHVESFYSNAIKVHNEDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCLPKREPLDVPDEPEDDQPRPRGDNFAVEKPNENSNLKCVTNVAGLASLVISLFPKLIIKNPQVLRPLLNVSWGYLFGSTFWLCFFSEVGLLRSLKNMKGVPLPESASEAKKLLEEMKNSEGDFNRRSLDFQYFFSLATLFSGILLLSTVKLANHNLQLRLSSSVVVITSLLNSLYLHNKVHNLKSKKESLYNDFIANPKNEKTVADLKKNKKEFHIFHGLSVLSLYVSFFGLTPYIFT

SEQ ID NO: 46 - DNA, 3210 bp, Artificial Sequence
  Description of Artificial Sequence Synthetic polynucleotide
gctgacaacaacagctacagcttcgagatcgtgaaccgcagcacctggctgaacatcgccgagcgcatcttcaagggcaacgcccccttcaacttcaccatcatcccctacaactacgtgaacaacagcaccgaggagaacaacaacaaggacagcgtgctgctgatcagcaagaacctgaagaacagcagcaaccccgtggacgagaacaaccacatcatcgacagcaccaagaagaacaccagcaacaacaacaacaacaacagcaacatcgtgggcatctacgagagccaggtgcacgaggagaagatcaaggaggacaacacccgccaggacaacatcaacaagaaggagaacgagatcatcaacaacaaccaccagatccccgtgagcaacatcttcagcgagaacatcgacaacaacaagaactacatcgagagcaactacaagagcacctacaacaacaaccccgagctgatccacagcaccgacttcatcggcagcaacaacaaccacaccttcaacttcctgagccgctacaacaacagcgtgctgaacaacatgcagggcaacaccaaggtgcccggcaacgtgcccgagctgaaggcccgcatcttcagcgaggaggagaacaccgaggtggagagcgccgagaacaaccacaccaacagcctgaaccccaacgagagctgcgaccagatcatcaagctgggcgacatcatcaacagcgtgaacgagaagatcatcagcatcaacagcaccgtgaacaacgtgctgtgcatcaacctggacagcgtgaacggcaacggcttcgtgtggaccctgctgggcgtgcacaagaagaagcccctgatcgaccccagcaacttccccaccaagcgcgtgacccagagctacgtgagccccgacatcagcgtgaccaaccccgtgcccatccccaagaacagcaacaccaacaaggacgacagcatcaacaacaagcaggacggcagccagaacaacaccaccaccaaccacttccccaagccccgcgagcagctggtgggcggcagcagcatgctgatcagcaagatcaagccccacaagcccggcaagtacttcatcgtgtacagctactaccgccccttcgaccccacccgcgacaccaacacccgcatcgtggagctgaacgtgcaggaggaggacgacaaggaggtggagcccgaggaggagaaggagaacgagcagaagaaggaggagcaggaggagaacaacctggaggcccccagcaagaccctgatgaagggcgtgaagaccaacatctacttcctgagcaccaaggagcgcatcgaggccctgatgtgctacaactacatcagcaacgccatcatcttcgagaagggcaagttcctgcgctacatcttcatgaacaacgtgaacaacatcatcgtgaacgagcacatgatcaacatgctgtgcaagaaggagaagatcaagtacatcctgagcagcaacagcatcatcatcgagagcaacgacttcatcaagcccctgatcatcgagttcgacagcaacatcagcaagaagatcttcgtgaagcacctgaagatggtggacagcttcaagctggacgacaagctgtaccgcgagtacctgaacgacctgagcgagcacgagcgcgaccgcctgaagcacgtggagagcttctacagcaacgccatcaaggtgcacaacgaggacgtgcagaacaacatcgtggacgagatcaagtaccgcgaggaggtgtgcaacgacgaggtggacctgtacctgctgatggactgcagcggcagcatccgccgccacaactgggtgaaccacgccgtgcccctggccatgaagctgatccagcagctgaacctgaacgacaacgccatccacctgtacgccagcgtgttcagcaacaacgcccgcgagatcatccgcctgcacagcgacgccagcaagaacaaggagaaggccctgatcatcatcaagagcctgctgagcaccaacctgccctacggcaagaccaacctgaccgacgccctgctgcaggtgcgcaagcacctgaacgaccgcatcaaccgcgagaacgccaaccagctggtggtgatcctgaccgacggcatccccgacagcatccaggacagcctgaaggagagccgcaagctgagcgaccgcggcgtgaagatcgccgtgttcggcatcggccagggcatcaacgtggccttcaaccgcttcctggtgggctgccaccccagcgacggcaagtgcaacctgtacgccgacagcgcctgggagaacgtgaagaacgtgatcggccccttcatgaaggccgtgtgcgtggaggtggagaagaccgccagctgcggcgtgtgggacgagtggagcccctgcagcgtgacctgcggcaagggcacccgcagccgcaagcgcgagatcctgcacgagggctgcaccagcgagctgcaggagcagtgcgaggaggagcgctgcctgcccaagcgcgagcccctggacgtgcccgacgagcccgaggacgaccagccccgcccccgcggcgacaacttcgccgtggagaagcccaacgagaacagcaacctgaagtgcgtgaccaacgtggccggcctggccagcctggtgatcagcctgttccccaagctgatcatcaagaacccccaggtgctgcgccccctgctgaacgtgagctggggctacctgttcggcagcaccttctggctgtgcttcttcagcgaggtgggcctgctgcgcagcctgaagaacatgaagggcgtgcccctgcccgagagcgccagcgaggccaagaagctgctggaggagatgaagaacagcgagggcgacttcaaccgccgcagcctggacttccagtacttcttcagcctggccaccctgttcagcggcatcctgctgctgagcaccgtgaagctggccaaccacaacctgcagctgcgcctgagcagcagcgtggtggtgatcaccagcctgctgaacagcctgtacctgcacaacaaggtgcacaacctgaagagcaagaaggagagcctgtacaacgacttcatcgccaaccccaagaacgagaagaccgtggccgacctgaagaagaacaagaaggagttccacatcttccacggcctgagcgtgctgagcctgtacgtgagcttcttcggcctgaccccctacatcttcacc

SEQ ID NO: 47 - protein, 1069 aa, Artificial Sequence
  Description of Artificial Sequence Synthetic polypeptide
DNNSYSFEIVNRSTWLNIAERIFKGNAPFNFTIIPYNYVNNSTEENNNKDSVLLISKNLKNSSNPVDENNHIIDSTKKNTSNNNNNNSNIVGIYESQVHEEKIKEDNTRQDNINKKENEIINNNHQIPVSNIFSENIDNNKNYIESNYKSTYNNNPELIHSTDFIGSNNNHTFNFLSRYNNSVLNNMQGNTKVPGNVPELKARIFSEEENTEVESAENNHTNSLNPNESCDQIIKLGDIINSVNEKIISINSTVNNVLCINLDSVNGNGFVWTLLGVHKKKPLIDPSNFPTKRVTQSYVSPDISVTNPVPIPKNSNTNKDDSINNKQDGSQNNTTTNHFPKPREQLVGGSSMLISKIKPHKPGKYFIVYSYYRPFDPTRDTNTRIVELNVQEEDDKEVEPEEEKENEQKKEEQEENNLEAPSKTLMKGVKTNIYFLSTKERIEALMCYNYISNAIIFEKGKFLRYIFMNNVNNIIVNEHMINMLCKKEKIKYILSSNSIIIESNDFIKPLIIEFDSNISKKIFVKHLKMVDSFKLDDKLYREYLNDLSEHERDRLKHVESFYSNAIKVHNEDVQNNIVDEIKYREEVCNDEVDLYLLMDCSGSIRRHNWVNHAVPLAMKLIQQLNLNDNAIHLYASVFSNNAREIIRLHSDASKNKEKALIIIKSLLSTNLPYGKTNLTDALLQVRKHLNDRINRENANQLVVILTDGIPDSIQDSLKESRKLSDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCLPKREPLDVPDEPEDDQPRPRGDNFAVEKPNENSNLKCVTNVAGLASLVISLFPKLIIKNPQVLRPLLNVSWGYLFGSTFWLCFFSEVGLLRSLKNMKGVPLPESASEAKKLLEEMKNSEGDFNRRSLDFQYFFSLATLFSGILLLSTVKLANHNLQLRLSSSVVVITSLLNSLYLHNKVHNLKSKKESLYNDFIANPKNEKTVADLKKNKKEFHIFHGLSVLSLYVSFFGLTPYIFT

SEQ ID NO: 48 - DNA, 18967 bp, Artificial Sequence
  Description of Artificial Sequence Synthetic polynucleotide
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 49 - DNA, 19795 bp, Artificial Sequence
  Description of Artificial Sequence Synthetic polynucleotide
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 50 - DNA, 20212 bp, Artificial Sequence
  Description of Artificial Sequence Synthetic polynucleotide
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatgtctattatgggactgaaggtgaacgtgagcgcaatctttatggctgtgctgctgaccctgcagactcctaccgggctgttccaggagtaccagtgttatgggtctagcagtaatactagggtgctgaacgaactgaattatgataacgcagggaccaatctgtacaacgagctggaaatgaattactacggaaagcaggagaactggtatagcctgaagaagaattcccgatctctgggcgagaacgatgatgggaataacgaggacaacgagaagctgaggaaacccaagcataagaagctgaagcagcccgccgatgggaacccagaccccaatgccaatcccaatgtggacccaaacgctaaccccaatgtcgatccaaatgccaacccaaatgtggacccaaatgcaaatcctaatgctaacccaaatgccaacccaaatgctaaccctaacgcaaatcccaatgcaaaccctaatgccaatcctaacgcaaatcctaacgcaaacccaaacgctaatccaaacgcaaatcctaacgctaatcctaacgctaatccaaacgccaatccaaacgccaatcctaacgccaatcccaacgcaaatccaaacgtggaccctaatgccaaccctaatgctaatccaaacgcaaaccctaatgcaaacccaaatgcaaaccccaatgcaaatcccaacgctaatcccaacgccaaccccaatgccaaccccaatgctaaccccaatgctaaccccaacgccaaccctaacgctaacccaaatgctaaccccaacgcaaaccccaacgccaacccaaacgccaacccaaatgcaaatcccaataagaacaatcagggtaatggacaggggcacaacatgccaaatgaccctaatcgaaacgtggacgagaacgccaatgcaaattcagctgtgaagaacaataataacgaagagccctctgataaacatatcaaggagtatctgaataagatccagaatagtctgtctaccgagtggtctccctgcagcgtgacatgtggaaatggaattcaggtccggattaagccagggtccgccaacaagcctaaggacgaactggactatgctaacgacattgagaagaagatttgctagtaagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 51 - DNA, 20101 bp, Artificial Sequence
  Description of Artificial Sequence Synthetic polynucleotide
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatggccaagaagtgtaccatactggtcgttgcgtcacttctgttggtcaattctctgctcccaggctatggacagaacaaatccattcaggcacagaggaacctcaacgaactctgctacaatgaagggaatgacaataagctgtatcacgtgctgaattccaagaacggcaaaatctacaatcgcaacacagtaaatcggttgcttgccgatgcacccgagggtaagaagaatgaaaagaagaatgagaaaatcgagcgcaacaacaagcttaaacagccaccgcctcctcctaacccaaatgacccaccgccacccaatccaaacgacccaccgcctcccaaccctaacgatcctccaccgcccaaccctaatgacccaccacctcccaatgcaaacgatccaccccctcctaacgctaacgaccctgctccacccaacgctaacgatcccgcgccccccaatgccaacgaccccgcaccacctaatgccaacgatcccgccccgcccaatgctaatgatcctccaccacccaacccaaacgaccctgcccctcctaatgctaacgatccaccacctcccaatccgaatgatcccgctccacctcaggggaacaacaaccctcagccccaacctagaccacagccgcagccccaaccccaaccccagccccagcctcaaccccagccccagccacgtccgcagcctcagcctcaacctggaggcaacaataacaacaagaataacaacaacgacgacagctacattccaagtgccgagaaaattctggagtttgttaagcagatccgagacagcataaccgaagaatggtcacagtgtaacgtgacgtgtggatctggcatcagagtgaggaaacggaagggttccaataagaaagcagaggatctgactctggaggacattgatacagagatctgcaaaatggacaaatgcagctctatcttcaacatcgtgagtaatagcctcgggtttgtgattctgctggtcctggtgttcttcaattgagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 52 - DNA, 22321 bp, Artificial Sequence
  Description of Artificial Sequence Synthetic polynucleotide
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatgtctattatgggactgaaggtgaacgtgagcgcaatctttatggctgtgctgctgaccctgcagactcctaccggggaaaatgcaataaaaaaaacgaagaatcaagaaaataatctgacgttactaccaataaagagcactgaagaagaaaaagatgatataaaaaatggaaaggatataaaaaaagaaattgataatgataaagagaatataaaaacaaataatgctaaagatcattcaacatatataaaatcatatttgaatacaaatgtaaatgatggtttaaaatatttgtttattccttctcataattcttttataaaaaaatattctgtatttaatcaaataaatgatggcatgttattaaatgaaaaaaatgatgtgaaaaataatgaagactataaaaatgtggattataaaaatgttaattttttacaatatcattttaaagagttatcaaattataacattgcaaattctattgatattttacaagaaaaagaaggacatttggattttgttataatacctcattatacttttttagattattataaacatttatcttataattctatatatcataagtcctctacatatggaaagtgtatagctgtagatgcttttattaagaaaataaatgaaacatatgacaaagtgaaaagtaaatgtaatgatataaagaatgatttaattgcaactataaaaaaattagagcatccttatgatataaataataagaatgatgattcctatagatatgatatatctgaagaaatcgatgataaatctgaagagacagatgatgaaaccgaagaggtagaagatagtatacaagatacagatagtaatcatactccttcaaataaaaaaaaaaatgatcttatgaatagaacgtttaaaaagatgatggatgaatataatacaaaaaaaaaaaaattaattaaatgtataaaaaaccatgagaatgattttaataaaatatgtatggatatgaaaaattatggtacaaacctttttgaacaactttcatgttacaataataatttctgtaatacaaacggaataagatatcattatgatgaatatattcataaattaatattatctgttaaatcaaaaaacttaaataaagacctatcagatatgacaaatattttacaacaaagtgaattattattaaccaatttaaataaaaaaatgggttcctatatatatattgatacaataaaatttatacataaagaaatgaaacatatttttaacagaattgaatatcatacaaaaataataaacgataaaactaaaataattcaagacaaaattaaattaaatatatggagaacatttcaaaaagatgaattattaaaaagaattttagacatgtcaaatgaatattctttatttattactagtgatcatttaagacaaatgttatataatacattctattcaaaagaaaaacatttaaataatatatttcatcatttaatttatgtactacaaatgaagttcaatgatgtcccaattaaaatggaatattttcaaacatataaaaaaaataaaccacttacacaaaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctagtgattcgaactacagctcaacttacctgccaaccccatgccagtcgacccaactagtacaacctaaatccattataaaaaacttaggaaccaggtccacacagccgccagcccatcaaccatccactcccacgattggaggccggccatgtctattatgggactgaaggtgaacgtgagcgcaatctttatggctgtgctgctgaccctgcagactcctaccgggctgttccaggagtaccagtgttatgggtctagcagtaatactagggtgctgaacgaactgaattatgataacgcagggaccaatctgtacaacgagctggaaatgaattactacggaaagcaggagaactggtatagcctgaagaagaattcccgatctctgggcgagaacgatgatgggaataacgaggacaacgagaagctgaggaaacccaagcataagaagctgaagcagcccgccgatgggaacccagaccccaatgccaatcccaatgtggacccaaacgctaaccccaatgtcgatccaaatgccaacccaaatgtggacccaaatgcaaatcctaatgctaacccaaatgccaacccaaatgctaaccctaacgcaaatcccaatgcaaaccctaatgccaatcctaacgcaaatcctaacgcaaacccaaacgctaatccaaacgcaaatcctaacgctaatcctaacgctaatccaaacgccaatccaaacgccaatcctaacgccaatcccaacgcaaatccaaacgtggaccctaatgccaaccctaatgctaatccaaacgcaaaccctaatgcaaacccaaatgcaaaccccaatgcaaatcccaacgctaatcccaacgccaaccccaatgccaaccccaatgctaaccccaatgctaaccccaacgccaaccctaacgctaacccaaatgctaaccccaacgcaaaccccaacgccaacccaaacgccaacccaaatgcaaatcccaataagaacaatcagggtaatggacaggggcacaacatgccaaatgaccctaatcgaaacgtggacgagaacgccaatgcaaattcagctgtgaagaacaataataacgaagagccctctgataaacatatcaaggagtatctgaataagatccagaatagtctgtctaccgagtggtctccctgcagcgtgacatgtggaaatggaattcaggtccggattaagccagggtccgccaacaagcctaaggacgaactggactatgctaacgacattgagaagaagatttgcaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctagtaagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 53 - DNA, 21937 bp, Artificial Sequence
  Description of Artificial Sequence Synthetic polynucleotide
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatgtcaatcatgggactgaaggtgaacgtcagcgcaatcttcatggccgtgctgctgactctgcagaccccaacaggcgaaaatgcaataaaaaaaacgaagaatcaagaaaataatctgacgttactaccaataaagagcactgaagaagaaaaagatgatataaaaaatggaaaggatataaaaaaagaaattgataatgataaagagaatataaaaacaaataatgctaaagatcattcaacatatataaaatcatatttgaatacaaatgtaaatgatggtttaaaatatttgtttattccttctcataattcttttataaaaaaatattctgtatttaatcaaataaatgatggcatgttattaaatgaaaaaaatgatgtgaaaaataatgaagactataaaaatgtggattataaaaatgttaattttttacaatatcattttaaagagttatcaaattataacattgcaaattctattgatattttacaagaaaaagaaggacatttggattttgttataatacctcattatacttttttagattattataaacatttatcttataattctatatatcataagtcctctacatatggaaagtgtatagctgtagatgcttttattaagaaaataaatgaaacatatgacaaagtgaaaagtaaatgtaatgatataaagaatgatttaattgcaactataaaaaaattagagcatccttatgatataaataataagaatgatgattcctatagatatgatatatctgaagaaatcgatgataaatctgaagagacagatgatgaaaccgaagaggtagaagatagtatacaagatacagatagtaatcatactccttcaaataaaaaaaaaaatgatcttatgaatagaacgtttaaaaagatgatggatgaatataatacaaaaaaaaaaaaattaattaaatgtataaaaaaccatgagaatgattttaataaaatatgtatggatatgaaaaattatggtacaaacctttttgaacaactttcatgttacaataataatttctgtaatacaaacggaataagatatcattatgatgaatatattcataaattaatattatctgttaaatcaaaaaacttaaataaagacctatcagatatgacaaatattttacaacaaagtgaattattattaaccaatttaaataaaaaaatgggttcctatatatatattgatacaataaaatttatacataaagaaatgaaacatatttttaacagaattgaatatcatacaaaaataataaacgataaaactaaaataattcaagacaaaattaaattaaatatatggagaacatttcaaaaagatgaattattaaaaagaattttagacatgtcaaatgaatattctttatttattactagtgatcatttaagacaaatgttatataatacattctattcaaaagaaaaacatttaaataatatatttcatcatttaatttatgtactacaaatgaagttcaatgatgtcccaattaaaatggaatattttcaaacatataaaaaaaataaaccacttacacaatgattcgaactacagctcaacttacctgccaaccccatgccagtcgacccaactagtacaacctaaatccattataaaaaacttaggaaccaggtccacacagccgccagcccatcaaccatccactcccacgattggaggccggccatgtctattatgggactgaaggtgaacgtgagcgcaatctttatggctgtgctgctgaccctgcagactcctaccgggctgttccaggagtaccagtgttatgggtctagcagtaatactagggtgctgaacgaactgaattatgataacgcagggaccaatctgtacaacgagctggaaatgaattactacggaaagcaggagaactggtatagcctgaagaagaattcccgatctctgggcgagaacgatgatgggaataacgaggacaacgagaagctgaggaaacccaagcataagaagctgaagcagcccgccgatgggaacccagaccccaatgccaatcccaatgtggacccaaacgctaaccccaatgtcgatccaaatgccaacccaaatgtggacccaaatgcaaatcctaatgctaacccaaatgccaacccaaatgctaaccctaacgcaaatcccaatgcaaaccctaatgccaatcctaacgcaaatcctaacgcaaacccaaacgctaatccaaacgcaaatcctaacgctaatcctaacgctaatccaaacgccaatccaaacgccaatcctaacgccaatcccaacgcaaatccaaacgtggaccctaatgccaaccctaatgctaatccaaacgcaaaccctaatgcaaacccaaatgcaaaccccaatgcaaatcccaacgctaatcccaacgccaaccccaatgccaaccccaatgctaaccccaatgctaaccccaacgccaaccctaacgctaacccaaatgctaaccccaacgcaaaccccaacgccaacccaaacgccaacccaaatgcaaatcccaataagaacaatcagggtaatggacaggggcacaacatgccaaatgaccctaatcgaaacgtggacgagaacgccaatgcaaattcagctgtgaagaacaataataacgaagagccctctgataaacatatcaaggagtatctgaataagatccagaatagtctgtctaccgagtggtctccctgcagcgtgacatgtggaaatggaattcaggtccggattaagccagggtccgccaacaagcctaaggacgaactggactatgctaacgacattgagaagaagatttgctaggcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 54 - DNA, 22981 bp, Artificial Sequence
  Description of Artificial Sequence Synthetic polynucleotide
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatggccaagaagtgtaccatactggtcgttgcgtcacttctgttggtcaattctctgctcccaggctatggacagaacaaatccattcaggcacagaggaacctcaacgaactctgctacaatgaagggaatgacaataagctgtatcacgtgctgaattccaagaacggcaaaatctacaatcgcaacacagtaaatcggttgcttgccgatgcacccgagggtaagaagaatgaaaagaagaatgagaaaatcgagcgcaacaacaagcttaaacagccaccgcctcctcctaacccaaatgacccaccgccacccaatccaaacgacccaccgcctcccaaccctaacgatcctccaccgcccaaccctaatgacccaccacctcccaatgcaaacgatccaccccctcctaacgctaacgaccctgctccacccaacgctaacgatcccgcgccccccaatgccaacgaccccgcaccacctaatgccaacgatcccgccccgcccaatgctaatgatcctccaccacccaacccaaacgaccctgcccctcctaatgctaacgatccaccacctcccaatccgaatgatcccgctccacctcaggggaacaacaaccctcagccccaacctagaccacagccgcagccccaaccccaaccccagccccagcctcaaccccagccccagccacgtccgcagcctcagcctcaacctggaggcaacaataacaacaagaataacaacaacgacgacagctacattccaagtgccgagaaaattctggagtttgttaagcagatccgagacagcataaccgaagaatggtcacagtgtaacgtgacgtgtggatctggcatcagagtgaggaaacggaagggttccaataagaaagcagaggatctgactctggaggacattgatacagagatctgcaaaatggacaaatgcagctctatcttcaacatcgtgagtaatagcctcgggtttgtgattctgctggtcctggtgttcttcaattgagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacgatgtctattatgggactgaaggtgaacgtgagcgcaatctttatggctgtgctgctgaccctgcagactcctaccggggacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattccctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacagctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaacaggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggttggggcgagtatgagccacttgcagtgtctgacatctgttgctggtctgtcctctatcgtcatgtcaatgttccccaaactcattgccaataatccttccctgtttagaccactgctcaacatttcctggggatatctgttcggaagcactgtatggctgtgcttcttcagtgagattgggttggtcaggagaatcaatgctcctaaacggaagaatctgccagagaatgcagaacaagccaaagaacagctgaaggagatcaagaacaacgaaggcgattttaaccgacgcaatatcgacttcaagtacttctttagcctttccacaatcttctctagcatactgctgcttagcacagtgaaactcgccaacaacaatctgcagttgaggatctgttccaccattgtgtcactgagttgcatactgaacaatatgtactttcagaacaagatacactcacttgcactgaagaaagagagtctctttaaggacatgatcgatcgtccgaaagataccactattctggtgaacctgaagaagaacaagaccgactttcacatccatcatggcctttctctgctcttgctctatagcagcttctttggcctcactccctacattttcacgtgagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 55 - DNA, 22906 bp, Artificial Sequence
  Description of Artificial Sequence Synthetic polynucleotide
gcggccgctaatacgactcactatagggccaactttgtttggtctgatgagtccgtgaggacgaaacccggagtcccgggtcaccaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacgatggccaagaagtgtaccatactggtcgttgcgtcacttctgttggtcaattctctgctcccaggctatggacagaacaaatccattcaggcacagaggaacctcaacgaactctgctacaatgaagggaatgacaataagctgtatcacgtgctgaattccaagaacggcaaaatctacaatcgcaacacagtaaatcggttgcttgccgatgcacccgagggtaagaagaatgaaaagaagaatgagaaaatcgagcgcaacaacaagcttaaacagccaccgcctcctcctaacccaaatgacccaccgccacccaatccaaacgacccaccgcctcccaaccctaacgatcctccaccgcccaaccctaatgacccaccacctcccaatgcaaacgatccaccccctcctaacgctaacgaccctgctccacccaacgctaacgatcccgcgccccccaatgccaacgaccccgcaccacctaatgccaacgatcccgccccgcccaatgctaatgatcctccaccacccaacccaaacgaccctgcccctcctaatgctaacgatccaccacctcccaatccgaatgatcccgctccacctcaggggaacaacaaccctcagccccaacctagaccacagccgcagccccaaccccaaccccagccccagcctcaaccccagccccagccacgtccgcagcctcagcctcaacctggaggcaacaataacaacaagaataacaacaacgacgacagctacattccaagtgccgagaaaattctggagtttgttaagcagatccgagacagcataaccgaagaatggtcacagtgtaacgtgacgtgtggatctggcatcagagtgaggaaacggaagggttccaataagaaagcagaggatctgactctggaggacattgatacagagatctgcaaaatggacaaatgcagctctatcttcaacatcgtgagtaatagcctcgggtttgtgattctgctggtcctggtgttcttcaattgagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtctaccctccatcattgttataaaaaacttaggaaccaggtccacacagccgccagcccatcaacgcgtacgatggacaatgacatctacagcttcgacattgtcaatgagacaaattggctgaagatcgccaagaacatcttcaaaggcaagtctcctagcaatttcacgatcataccgtttaacaataccggtagttctaacgataacgagtcaaacaaggaggaatcagtactgctgatcagaaagaagatcaaaagcaacaagaatcacgatagttccatcattagtggtgacactgttaacggggacattagtgacctgaattatacggctagcaacttttccgataactctgaggacatagaagataaccagaaatatcccacaaccagctacaatagtttcaaccatctcaattccctcactggggacgagaaatgtggtaaaagcctgaagctcggcaacatcagcaatcagacaaaccaggaaaccataacccaaagcctgtcagttggagagattctgtgcattgacctcgaagggaatgcaggaacaggctatctgtgggtgttgctgggcatacacaaggatgagccaatcataaaccccgagaacttcccaaccaaactgacaaagaagtctttcttttccgaggaaataagtgtgactcagccaaagaagtacaagatcgatgagcatgattcttcaaagaatgtgaatcgcgaaatcgaaagccctgaacagaaggagtccgactcaaagcccaagaaacctcagatgcaacttcttggaggaccagatcggatgaggtcagtcatcaaaggacacaaacctggcaaatattacattgtgtactcttactatcgaccgttttctcccacttctggggcgaacactaaaatcatttacgtcacagtacagctgatcgaagagaaagaggaaatcacccctgagaacaaaaccctcataatgccctctaaaactctgatgaagggcattaagaccaacatttacttcctgtcaaacaaggaaaagatccaagtgcttatgtgctataactacaagtgtgatgccgttgtgttcgagaaagacacctttctgcgctatctctacatcaagagcatcaataatatcatcctgaacgaaagaatgattgaacagttgtgcaagaacgaaaacctgaagtacatccttgcctgcaacagcatagtggttgaatcaagcgacttcatcaaacccctgatcattgagtttgagtcatccacttccaagaacatcttcgtaaagcacattaagcacaatagccagaaagaaatggacatcaacaagttcaacgagtatatgcgggatctcaaaagcaatgagaagctcagactgaagaaagtcgagcgattccactctattaatctggcagccaagaaacaggagattctggacgaaatcaagtactctgaggaggtctgcaacgaacaaatcgatctccacattctgctggatgggagtggcagcataggtcactctaactggatcagtcacgtgatacccatgctgacaacccttgtggacaatttgaacatcagccgcgatgagatcaatatctccatgaccttgttctccacttatgccagggaacttgtgagacttaagagatatgggtctacaagcaaagccagtctgaggttcatcatcgcgcaactccagaataactattctcctcatggaacgacaaatctgactagcgccctgttgaatgtggacaatctcattcagaagaaaatgaatcgccctaatgccattcagctcgtgattatccttactgacggcatccctaacaatctgaagaagtccactactgttgtcaaccagctgaagaagaaggacgtcaatgtcgctattattggtgttggcgccggagtaaacaatatgtttaaccgtatattggtaggatgtggaaaacttgggccttgtccctactactcttatggctcttgggatcaagcacaaaccatgatcaaaccatttctctcaaaggtctgtcaggaagtggagaaagtggcactgtgcggtaagtgggaggagtggagtgagtgttcaaccacttgcgacaacggaacgaaaataaggaagcgaaaggttggggcgagtatgagccacttgcagtgtctgacatctgttgctggtctgtcctctatcgtcatgtcaatgttccccaaactcattgccaataatccttccctgtttagaccactgctcaacatttcctggggatatctgttcggaagcactgtatggctgtgcttcttcagtgagattgggttggtcaggagaatcaatgctcctaaacggaagaatctgccagagaatgcagaacaagccaaagaacagctgaaggagatcaagaacaacgaaggcgattttaaccgacgcaatatcgacttcaagtacttctttagcctttccacaatcttctctagcatactgctgcttagcacagtgaaactcgccaacaacaatctgcagttgaggatctgttccaccattgtgtcactgagttgcatactgaacaatatgtactttcagaacaagatacactcacttgcactgaagaaagagagtctctttaaggacatgatcgatcgtccgaaagataccactattctggtgaacctgaagaagaacaagaccgactttcacatccatcatggcctttctctgctcttgctctatagcagcttctttggcctcactccctacattttcacgtgagcgcgcagcgcttagacgtctcgcgatcgatgctagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatgcggccgcgggccctatggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatgcttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagccaccgcggtg

SEQ ID NO: 56 - DNA, 1776 bp, Artificial Sequence
  Description of Artificial Sequence Synthetic polynucleotide
atgagtattatgggactgaaggtgaacgtgtccgctatctttatggctgtgctgctgaccctgcagactccaactggcgaaaatgctatcaagaagaccaagaaccaggagaacaatctggccctgctgcctatcaagagcacagaggaggagaaggacgatatcaagaatggcaaggacatcaagaaggagatcgacaatgataaggagaacatcaagaccaacaatgccaaggaccacagcacatacatcaagtcctatctgaataccaacgtgaatgatggcctgaagtacctgttcatcccatcccacaactcttttatcaagaagtattccgtgttcaaccagatcaatgacggcatgctgctgaacgagaagaatgatgtgaagaacaatgaggactacaagaacgtggattataagaacgtgaatttcctgcagtaccactttaaggagctgagcaactataatatcgccaattccatcgacatcctgcaggagaaggagggccacctggattttgtgatcatcccccactacacattcctggactactataagcacctgagctacaactccatctatcacaagagctccacatacggcaagtgtatcgccgtggacgccttcatcaagaagatcaatgaggcctacgacaaggtgaagagcaagtgcaatgacatcaagaacgatctgatcgccaccatcaagaagctggagcacccatacgacatcaacaataagaacgacgattcctacagatatgatatctctgaggagatcgacgataagtccgaggagacagacgatgagacagaggaggtggaggactctatccaggacaccgattctaaccacgcccccagcaacaagaagaagaacgacctgatgaatagggccttcaagaagatgatggatgagtacaataccaagaagaagaagctgatcaagtgtatcaagaaccacgagaatgacttcaacaagatctgcatggacatgaagaactacggcacaaatctgtttgagcagctgtcctgttataacaataacttctgcaacaccaatggcatcaggtaccactatgacgagtatatccacaagctgatcctgtctgtgaagagcaagaacctgaataaggacctgtctgatatgacaaacatcctgcagcagagcgagctgctgctgaccaacctgaataagaagatgggctcttacatctatatcgatacaatcaagtttatccacaaggagatgaagcacatcttcaaccggatcgagtaccacaccaagatcatcaatgacaagacaaagatcatccaggataagatcaagctgaacatctggcggacctttcagaaggacgagctgctgaagcgcatcctggatatgtccaacgagtactctctgttcatcacaagcgaccacctgcgccagatgctgtacaataccttctacagcaaggagaagcacctgaataacatcttccaccacctgatctatgtgctgcagatgaagtttaacgatgtgcccatcaagatggagtacttccagacctataagaagaataagcctctgacccagaagggcctgtctagcacatctatcgtgtacatcctgatcgccgtgtgcctgggaggcctgatcggaatccctgccctgatctgctgttgcaggggcagatgcaacaagaagggcgagcaagtgggcatgagcaggcccggactgaagcctgacctgactgggacaagcaaatcctatgtgcggagcctgtaataa

SEQ ID NO: 57 - protein, 589 aa, Artificial Sequence
  Description of Artificial Sequence Synthetic polypeptide
SIMGLKVNVSAIFMAVLLTLQTPTGENAIKKTKNQENNLALLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQKGLSSTSIVYILIAVCLGGLIGIPALICCCRGRCNKKGEQVGMSRPGLKPDLTGTSKSYVRSL

SEQ ID NO: 58 - DNA, 1584 bp, Artificial Sequence
  Description of Artificial Sequence Synthetic polynucleotide
atgagtattatgggactgaaggtgaacgtgtccgctatctttatggctgtgctgctgaccctgcagactccaactggcgaaaatgctatcaagaagaccaagaaccaggagaacaatctggccctgctgcctatcaagagcacagaggaggagaaggacgatatcaagaatggcaaggacatcaagaaggagatcgacaatgataaggagaacatcaagaccaacaatgccaaggaccacagcacatacatcaagtcctatctgaataccaacgtgaatgatggcctgaagtacctgttcatcccatcccacaactcttttatcaagaagtattccgtgttcaaccagatcaatgacggcatgctgctgaacgagaagaatgatgtgaagaacaatgaggactacaagaacgtggattataagaacgtgaatttcctgcagtaccactttaaggagctgagcaactataatatcgccaattccatcgacatcctgcaggagaaggagggccacctggattttgtgatcatcccccactacacattcctggactactataagcacctgagctacaactccatctatcacaagagctccacatacggcaagtgtatcgccgtggacgccttcatcaagaagatcaatgaggcctacgacaaggtgaagagcaagtgcaatgacatcaagaacgatctgatcgccaccatcaagaagctggagcacccatacgacatcaacaataagaacgacgattcctacagatatgatatctctgaggagatcgacgataagtccgaggagacagacgatgagacagaggaggtggaggactctatccaggacaccgattctaaccacgcccccagcaacaagaagaagaacgacctgatgaatagggccttcaagaagatgatggatgagtacaataccaagaagaagaagctgatcaagtgtatcaagaaccacgagaatgacttcaacaagatctgcatggacatgaagaactacggcacaaatctgtttgagcagctgtcctgttataacaataacttctgcaacaccaatggcatcaggtaccactatgacgagtatatccacaagctgatcctgtctgtgaagagcaagaacctgaataaggacctgtctgatatgacaaacatcctgcagcagagcgagctgctgctgaccaacctgaataagaagatgggctcttacatctatatcgatacaatcaagtttatccacaaggagatgaagcacatcttcaaccggatcgagtaccacaccaagatcatcaatgacaagacaaagatcatccaggataagatcaagctgaacatctggcggacctttcagaaggacgagctgctgaagcgcatcctggatatgtccaacgagtactctctgttcatcacaagcgaccacctgcgccagatgctgtacaataccttctacagcaaggagaagcacctgaataacatcttccaccacctgatctatgtgctgcagatgaagtttaacgatgtgcccatcaagatggagtacttccagacctataagaagaataagcctctgacccagtaa

SEQ ID NO: 59 - protein, 526 aa, Artificial Sequence
  Description of Artificial Sequence Synthetic polypeptide
SIMGLKVNVSAIFMAVLLTLQTPTGENAIKKTKNQENNLALLPIKSTEEEKDDIKNGKDIKKEIDNDKENIKTNNAKDHSTYIKSYLNTNVNDGLKYLFIPSHNSFIKKYSVFNQINDGMLLNEKNDVKNNEDYKNVDYKNVNFLQYHFKELSNYNIANSIDILQEKEGHLDFVIIPHYTFLDYYKHLSYNSIYHKSSTYGKCIAVDAFIKKINEAYDKVKSKCNDIKNDLIATIKKLEHPYDINNKNDDSYRYDISEEIDDKSEETDDETEEVEDSIQDTDSNHAPSNKKKNDLMNRAFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFNDVPIKMEYFQTYKKNKPLTQ

SEQ ID NO: 60 - protein, 28 aa, Artificial Sequence
  Description of Artificial Sequence Synthetic peptide
IHYKKLRNQVHTAASPSTIHSHDWRPAM

SEQ ID NO: 61 - protein, 404 aa, Artificial Sequence
  Description of Artificial Sequence Synthetic polypeptide
IHYKKLRNQVHTAASPSTIHSHDWRPAMSIMGLKVNVSAIFMAVLLTLQTPTGLFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKIC

SEQ ID NO: 62 - protein, 467 aa, Artificial Sequence
  Description of Artificial Sequence Synthetic polypeptide
IHYKKLRNQVHTAASPSTIHSHDWRPAMSIMGLKVNVSAIFMAVLLTLQTPTGLFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKGLSSTSIVYILIAVCLGGLIGIPALICCCRGRCNKKGEQVGMSRPGLKPDLTGTSKSYVRSL